(19) United States
(12) Patent Application Publication
(10) Pub. No.: US 2026/0085102 A1
(43) Pub. Date: Mar. 26, 2026

(54) TISSUE SPECIFIC SYNTHETIC PROMOTERS AND CIRCUITS AND USE THEREOF

(71) Applicant: YISSUM RESEARCH DEVELOPMENT COMPANY OF THE HEBREW UNIVERSITY OF JERUSALEM LTD., Jerusalem (IL)

(72) Inventors: Lior NISSIM, Mevaseret Zion (IL); Chaja KATZMAN, Jerusalem (IL); Tamar AMINOV, Ramat Gan (IL); Masha ZAITSEV-CHAIKOVSKY, Hadera (IL); Nir SKALKA, Rehovot (IL); Amit SHRAGA, Ganei Tikva (IL); Konstantin ROZENBERG, Rosh Haiin (IL); Chen SHIMONI, Holon (IL); Inbar SHTEINBERG, Sderot (IL)

(21) Appl. No.: 19/325,757

(22) Filed: Sep. 11, 2025

Related U.S. Application Data
(63) Continuation-in-part of application No. PCT/IL2024/050262, filed on Mar. 12, 2024.
(60) Provisional application No. 63/451,596, filed on Mar. 12, 2023.

(51) Int. Cl. C07K 14/54; A61K 48/00; A61P 35/00; C07K 14/55; C07K 14/705; C12N 15/86

(52) U.S. Cl. CPC C07K 14/5443; A61K 48/0058; A61P 35/00; C07K 14/55; C07K 14/70575; C12N 15/86; C12N 2740/15043; C12N 2830/60; C12N 2840/007

(57) ABSTRACT

Nucleic acid molecules configured to specifically express a sequence of interest in a cancer cell, comprising: a transcription factor binding region comprising 3-10 repeats of a transcription factor binding site, a minimal promoter and the sequence of interest operatively linked to the transcription factor binding region and minimal promoter, wherein the transcription factor is selected from SOX9, GATA10, TCF7L1, LEF1, MYBL2 and MYB are provided. Nucleic acid molecule systems and compositions comprising the nucleic acid molecules are also provided as are methods of treating cancer.

### REFERENCE TO AN ELECTRONIC SEQUENCE LISTING

The contents of the electronic sequence listing (HUJI-P-0139-US.xml; Size: 134,279 bytes; and Date of Creation: Sep. 11, 2025) is herein incorporated by reference in its entirety.

### CROSS REFERENCE TO RELATED APPLICATIONS

This application is a Continuation-in-part of International Patent Application No. PCT/IL2024/050262 filed Mar. 12, 2024, which claims the benefit of priority of U.S. Provisional Patent Application No. 63/451,596, filed Mar. 12, 2023, the contents of which are all incorporated herein by reference in their entirety.

### FIELD OF INVENTION

The present invention is in the field of synthetic expression circuits.

### BACKGROUND OF THE INVENTION

The global cancer burden in 2020 is estimated at 19.3 million new cases and 10 million deaths. In general, cancer can arise from any organ or cell type in the body, and there are more than 100 distinct types of cancer. Conventional anticancer treatments including surgery, chemotherapy and radiation therapy are typically non-specific approaches. Surgery can be used to treat solid tumors, but in many cases, it is only effective in the early stages of the disease and loses its effectiveness once the malignancy becomes metastatic. Indeed, many cancer types form metastatic tumors, and more aggressive combined treatment is needed, such as chemotherapy and radiation therapy. The efficacy of these treatments usually involves high doses of radiation and drugs to eliminate tumor cells, which often cause severe toxicities and additional damage to healthy cells. Therefore, more precise and personal treatment options are needed before selecting a patient's therapy.

Immunotherapy, advancing rapidly in the last decades, helps the immune system in its fight against cancer and represents an outstanding example of precision and personalized medicine (PPM). There are different approaches to this paradigm including immune checkpoint inhibitors that precisely block inhibitory processes and restore immune function, bispecific antibodies (bsAbs), tumor-infiltrating lymphocytes (TILs), T-cell receptor (TCR) engineered T-cells, and chimeric antigen receptor (CAR) T-cells and natural killer (NK) cells that target cancer cells through tumor antigen recognition.

Among the above, adoptive T cell transfer, bispecific antibodies and immune checkpoint inhibitors (such as PD-1/PD-L1 and CTLA-4), have drawn most of the attention. For example, in recent years the FDA has approved immune checkpoint inhibitors (such as nivolumab, pembrolizumab and atezolizumab), for the treatment of patients with several colorectal cancer (CRC) and breast cancer indications. Unfortunately, many cancer patients cannot benefit from these immunotherapies, or acquire resistance to the treatment and relapse. Resistance can occur by increased expression levels of immune checkpoint proteins by the tumor or immune cells, involvement of immune suppressor cells (such as T regulatory (Tregs) CD4 cells), and escape through the change or loss of tumor-specific target antigens expressed by the tumor cells. Consequently, the ability of the immune system to specifically target the tumor cells is lost and prevents treatment efficiency. In addition, immune-modulatory therapies are often toxic due to their recognition of target proteins and antigens on normal tissues. There is a great need for an approach to overcome the lack of highly specific tumor surface antigens, immunotherapy drug resistance, tumor-mediated immunosuppression and toxicity.

### SUMMARY OF THE INVENTION

The present invention provides nucleic acid molecules configured to specifically express a sequence of interest in a cancer cell, comprising: a transcription factor binding region comprising 3-10 repeats of a transcription factor binding site, a minimal promoter and the sequence of interest operatively linked to the transcription factor binding region and minimal promoter, wherein the transcription factor is selected from SOX9, GATA10, TCF7L1, LEF1, MYBL2 and MYB. Nucleic acid molecule systems and compositions comprising the nucleic acid molecules are also provided as are methods of treating cancer.

According to a first aspect, there is provided a nucleic acid molecule configured to specifically express a sequence of interest in a cancer cell, the molecule comprising: a transcription factor binding region comprising 3-10 repeats of a transcription factor binding site, a minimal promoter, and the sequence of interest operatively linked to the transcription factor binding region and minimal promoter, wherein the transcription factor is selected from SOX9, GATA10, TCF7L1, LEF1, MYBL2 and MYB.

According to some embodiments, the transcription factor is SOX9 and the transcription factor binding site comprises NAACAAWRG (SEQ ID NO: 104) or its reverse complement.

According to some embodiments, the transcription factor is SOX9 and the transcription factor binding site comprises or consists of GAACAAAGGCCCCTTT (SEQ ID NO: 1) or its reverse complement.

According to some embodiments, the transcription factor is GATA10 and the transcription factor binding site comprises YBHCYBHDWTHW (SEQ ID NO: 106) or its reverse complement.

According to some embodiments, the transcription factor is GATA10 and the transcription factor binding site comprises or consists of TCCCTTTTATCT (SEQ ID NO: 2) or its reverse complement.

According to some embodiments, the transcription factor is TCF7L1 and the transcription factor binding site comprises YCTTTGATSTY (SEQ ID NO: 108) or its reverse complement.

According to some embodiments, the transcription factor is TCF7L1 and the transcription factor binding site comprises or consists of TGCCTTTGATGTT (SEQ ID NO: 3) or its reverse complement.

According to some embodiments, the transcription factor is LEF1 and the transcription factor binding site comprises NNASATCAAAGNNNN (SEQ ID NO: 110) or its reverse complement.

According to some embodiments, the transcription factor is LEF1 and the transcription factor binding site comprises or consists of AAAGATCAAAGGGTT (SEQ ID NO: 4) or its reverse complement.

According to some embodiments, the transcription factor is LEF1 and the transcription factor binding site comprises CCTTTGAT (SEQ ID NO: 112) or its reverse complement.

According to some embodiments, the transcription factor is LEF1 and the transcription factor binding site comprises or consists of TCCTTTGATTTGCT (SEQ ID NO: 5) or its reverse complement.

According to some embodiments, the transcription factor is MYBL2 and the transcription factor binding site comprises RACSGTTAACSKHY (SEQ ID NO: 113) or its reverse complement.

According to some embodiments, the transcription factor is MYBL2 and the transcription factor binding site comprises or consists of AACGGTTAACGGTT (SEQ ID NO: 6) or its reverse complement.

According to some embodiments, the transcription factor is MYB and the transcription factor binding site comprises NNDDCCGTTD (SEQ ID NO: 115) or its reverse complement.

According to some embodiments, the transcription factor is MYB and the transcription factor binding site comprises or consists of TTGACCGTTA (SEQ ID NO: 7) or its reverse complement.

According to some embodiments, the 3-10 repeats are all binding sites of the same transcription factor.

According to some embodiments, the 3-10 repeats are all identical.

According to some embodiments, the 3-10 repeats are 6-9 repeats.

According to some embodiments, the transcription factor binding region comprises a spacer of between 1-5 nucleotides between each repeat.

According to some embodiments, the spacer consists of 3 nucleotides.

According to some embodiments, the transcription factor binding region consists of between 100 and 150 nucleotides.

According to some embodiments, the transcription factor binding region consists of between 110 and 125 nucleotides.

According to some embodiments, the transcription factor is selected from SOX9, GATA10, TCF7L1, and LEF1 and the nucleic acid molecule is configured to specifically express the sequence of interest in a colorectal cancer cell.

According to some embodiments, the transcription factor is selected from SOX9, MYBL2 and MYB and the nucleic acid molecule is configured to specifically express the sequence of interest in a breast cancer cell.

According to some embodiments, the transcription factor binding region comprises any one of SEQ ID NO: 8-14.

According to some embodiments, the minimal promoter is selected from the late adenoviral promoter and the YB_TATA promoter.

According to some embodiments, the specific expression comprises off target expression in non-cancerous cells below a predetermined threshold.

According to some embodiments, the specific expression is in colorectal cancer and the non-cancerous cells are colorectal cells or the specific expression is in breast cancer and the non-cancerous cells are breast cells.

According to some embodiments, the nucleic acid molecule further comprises a 5′ untranslated region (UTR) between the minimal promoter and the sequence of interest.

According to some embodiments, the sequence of interest encodes a therapeutic molecule configured to treat the cancer.

According to some embodiments, the molecule is an expression vector.

According to another aspect, the nucleic acid molecule system comprising:
a. a first nucleic acid molecule of the invention, wherein the sequence of interest encodes a first fusion protein comprising a DNA binding domain and a first protein binding domain;
b. a second nucleic acid molecule of the invention, wherein the sequence of interest encodes a second fusion protein comprising a transcriptional activation domain and a second protein binding domain; and
c. a third nucleic acid molecule comprising a polynucleotide operably linked to a transcriptional regulatory element, wherein the DNA binding domain binds to the transcriptional regulatory element;
wherein the first protein binding domain and the second protein binding domain are configured to bind to each other, and wherein the first nucleic acid molecule and the second nucleic acid molecule comprise repeats of different transcription factor binding sites.

According to some embodiments, the polynucleotide comprises an open reading frame encoding a protein or wherein the polynucleotide comprises a sequence of a non-coding RNA.

According to some embodiments, the protein is a reporter protein.

According to some embodiments, the protein or non-coding RNA is a therapeutic protein or therapeutic non-coding RNA.

According to some embodiments, the therapeutic protein is an anticancer protein, a toxin or a proapoptotic peptide.

According to some embodiments, the anticancer drug is an immunomodulatory protein selected from interleukin-15 (IL-15), IL-2, an anti-CD3e binding agent and a combination thereof.

According to some embodiments, the transcriptional regulatory element is a promoter.

According to some embodiments, recruitment of the transcriptional activation domain to the transcriptional regulatory element drives transcription of the polynucleotide.

According to some embodiments, the first and second protein binding domains are a DocS protein domain and a Coh2 protein domain or are a HrpS protein and a HrpR protein.

According to some embodiments, the first and second protein binding domains are a HrpS protein and a HrpR protein, and wherein the system further comprises a fourth nucleic acid molecule comprising a coding region encoding an HrpV protein operatively linked to a promoter configured to specifically express in non-cancerous cells.

According to some embodiments, the DNA binding domain is a Gal4-DNA binding domain and wherein the transcriptional regulatory element comprises a Gal4 binding site.

According to some embodiments, the transcriptional activation domain is the VP16 activation domain (VP16-AD) or VP64 activation domain (VP64-AD).

According to some embodiments, the first nucleic acid molecule comprises repeats of a LEF1 binding site and the second nucleic acid molecule comprises repeats of a SOX9 binding site or repeats of a TCF7L1 binding site.

According to some embodiments, the first nucleic acid molecule comprises repeats of SEQ ID NO: 5 and the second nucleic acid molecule comprises repeats of SEQ ID NO: 1.

According to some embodiments, the first nucleic acid molecule comprises SEQ ID NO: 12 and the second nucleic acid molecule comprises SEQ ID NO: 8.

According to some embodiments, the first nucleic acid molecule comprises repeats of SEQ ID NO: 4 and the second nucleic acid molecule comprises repeats of SEQ ID NO: 3.

According to some embodiments, the first nucleic acid molecule comprises SEQ ID NO: 11 and the second nucleic acid molecule comprises SEQ ID NO: 10.

According to some embodiments, the nucleic acid molecule system is for use in specifically expressing the polynucleotide in a colorectal cancer cell.

According to some embodiments, the first nucleic acid molecule comprises repeats of a MYBL2 binding site and the second nucleic acid molecule comprises repeats of a MYB binding site.

According to some embodiments, the first nucleic acid molecule comprises repeats of SEQ ID NO: 6 and the second nucleic acid molecule comprises repeats of SEQ ID NO: 7.

T According to some embodiments, the first nucleic acid molecule comprises SEQ ID NO: 13 and the second nucleic acid molecule comprises SEQ ID NO: 14.

According to some embodiments, the nucleic acid molecule system is for use in specifically expressing the polynucleotide in a breast cancer cell.

According to another aspect, there is provided a pharmaceutical composition comprising a nucleic acid molecule of the invention, a nucleic acid molecule system of the invention and a pharmaceutically acceptable carrier, excipient or adjuvant.

According to some embodiments, the pharmaceutical composition is formulated for systemic administration or intratumoral administration.

According to another aspect, there is provided a method of treating colorectal cancer in a subject in need thereof, the method comprising administering to the subject a nucleic acid molecule of the invention, a nucleic acid molecule system of the invention or a pharmaceutical composition of the invention, thereby treating colorectal cancer.

According to another aspect, there is provided a method of treating breast cancer in a subject in need thereof, the method comprising administering to the subject a nucleic acid molecule of the invention, a nucleic acid molecule system of any one of the invention or a pharmaceutical composition of the invention, thereby treating breast cancer.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A-1G: (1A) A heat map of relative EGFP expression from various synthetic promoters of the invention and control promoters in CRC cell lines, CRC patient samples, non-tumorigenic control cells and breast cancer cell lines. (1B) Relative EGFP expression from human PBMCs obtained from 3 healthy donors, showing that none of the synthetic promoters are expressed in the tested PBMC donors. The average expression in the three donor is shown (Mean with SEM, significance levels were calculated by using one-way ANOVA test; *** (PV<0.005)). (1C-1G) Scatter plots of mean fluorescent intensity (MFI) for EGFP expression of promoters (1C) pCTB3, (1D) pCTB4, (1E) pCTB5, (1F) pCTB6 and (1G) pCTB7 in the tested CRC cell lines, non-tumorigenic cell lines and breast cancer cell lines. A threshold value of an MFI of 9 was identified as indicating positive expression.

FIG. 2: A heat map of relative EGFP expression from various DPI combinations in CRC cell lines and non-tumorigenic control cells.

FIGS. 3A-3E: (3A-3B) Bar graphs of percent lethality of CRC cell lines (3A) HCT116 and (3B) SW480 after co culture with PBMCs expressing the various immunomodulator proteins or combinations. (3C-3D) Bar graphs of percent of PBMCs expressing activation markers CD25 and CD69 after coculture with (3C) HCT116 or (3D) SW480 cells expressing the indicated various immunomodulator proteins or combinations. (3E) Bar graph of IFNg secretion levels from PBMCs s after coculture with CRC cells expressing the various immunomodulator proteins or combinations.

FIG. 4A-4C: (4A-4B) Bar charts of MFI of EGFP from output constructs containing 3 or 8 Gal4 binding sites in (4A) HT29 CRC cells and (4B) HEK293 non-cancerous cells infected with various combinations of input constructs. (4C) Bar chart of log MFI from G8 output constructs in HT29 and HEK293 cells infected with various combinations of input constructs. (Mean with SEM, significance levels were calculated by using one-way ANOVA test; * (PV<0.05), ** (PV<0.01), *** (PV<0.005)).

FIG. 5: Diagram of embodiments of two circuits of the invention.

FIGS. 6A-6E: (6A-6D) Bar graphs of (6A) percent lethality, (6B) IFNg secretion, (6C) CD25 surface expression on immune cells and (6D) CD69 surface expression on immune cells from cocultures of CRC cell lines or non-tumorigenic colorectal cell lines or other non-tumorigenic cell lines with PBMCs or without PBMCs (untreated). PBMCs were either untreated or administered stimulatory antibodies (Abs) or the cell lines were infected with circuit 1. (6E) Micrographs of CCD-1096sk (non-cancerous) cells and SW480 (CRC cells) either untreated or infected with circuit 1 after coculture with PBMCs for 72 hours. (Mean with SEM, significance levels were calculated by using one-way ANOVA test; * (PV<0.05), ** (PV<0.01), *** (PV<0.005)).

FIG. 7A-7C: Bar graphs of (7A) percent lethality, (7B) CD69 surface expression on immune cells and (7C) CD25 surface expression on immune cells from cocultures of PDX cells from CRC patients with PBMCs. PBMCs were either untreated or administered stimulatory antibodies (Abs) or the PDX cells were infected with circuit 1 or circuit 2. (Mean with SEM, significance levels were calculated by using one-way ANOVA test; * (PV<0.05), ** (PV<0.01), *** (PV<0.005)).

FIG. 8: Diagram of an embodiment of a third circuit of the invention.

FIGS. 9A-9C: (9A) Line graph of tumor volume over time in mice administered untreated HCT116 cells (WT group) or HCT116 cells infected with circuit 3 (Treatment group). (9B-9C) Bar graphs of (9B) serum IL-2 levels over time and (9C) percent of tumor cells expressing EGFP in the excised tumor in mice administered untreated HCT116 cells (WT group) or HCT116 cells infected with circuit 3 (Treatment group). (Mean with SEM, significance levels were calculated by using one-way ANOVA test; * (PV<0.05), ** (PV<0.01), *** (PV<0.005)).

FIGS. 10A-10J: (10A-10B) Line graphs of tumor volume over time of (10A) untreated SW480 cells or (10B) SW480 cells infected with circuit 1 administered to mice with or without two subsequent administrations of naïve human PBMCs. (10C) Dot plot of tumor weights upon excision on day 31 from mice administered untreated SW480 cells or circuit 1-infected SW480 cells with or without subsequent naïve human PBMC administration. (10D) Bar graph of the average percent of cells in the excised tumors that were CD45 positive. (10E-10H) Scatter plot of markers for tumor infiltrating lymphocytes on day 31: (10E) CD45 positive cells, (10F) CD4 positive cells, (10G) CD8 positive cells, and (10H) CD14 positive cells. (10I-10J) Detection of (10I) IL-2 and (10J) INFg in blood serum at various time points. (Mean with SEM, significance levels were calculated by using one-way ANOVA test; * (PV<0.05), ** (PV<0.01), *** (PV<0.005)).

FIGS. 11A-11F: Heath maps of relative EGFP expression from (11A) various synthetic promoters of the invention and control promoters and (11B) from various DPI combinations in breast cancer cell lines and non-tumorigenic control cells. (11C-11F) Scatter plots of EGFP activity of promoters (11C) pCTB8, (11D) pCTB9, (11E) pCTB10, and (11F) pCTB11 in the tested breast cancer (BC) cell lines, and non-tumorigenic cell lines. A threshold value of an MFI of 9 was identified as indicating positive expression.

FIGS. 12A-12D: (12A) Bar graph of EGFP signal from various CRC cell lines, CRC primary cells or non-tumorigenic cell lines that were infected with adenovirus containing CMV-EGFP expression cassette or were untreated. (12B) Bar graph depicting flow cytometry results of percentage of cells expressing EGFP in SW480 and CCD1096Sk cells infected with adenovirus containing Circuit 1 with EGFP as the output. or CMV-EGFP. Two MOIs were tested for Circuit 1 and untreated cells were used as a negative control. (12C) Line chart of luminescence signal [RLU] over time in SW480 and CCD1096Sk cells infected with adenovirus containing Circuit 1 with Firefly-luciferase as the output or in untreated cells. Media without cells was used as background control. (12D) Bar graph of IL-2 in media of SW480 or CCD1096Sk cells untreated or infected with adenovirus containing Circuit 1 with IL-2 and anti-CD3e as the output.

FIGS. 13A-13B: (13A) Luminescence signal [(p/sec)/(cm²/sr)] from Balb/c Nude mice bearing SW480 CRC cell line tumors. Mice were injected by IT or IV with adenovirus containing either Circuit 1 or Circuit 2 with Firefly-luciferase as the output and imaged in IVIS. PSB administration was used as a control. (13B) Adenovirus DNA quantification (fold change) as detected via qPCR in tumors cells from mice injected by IT or repeated IV (days 0, 7) of either PBS or adenovirus containing Circuit 1 with IL-2 and anti-CD3e as the output. Mean, significance levels were calculated by using multiple unpaired t-test; * (PV<0.05), ** (PV<0.01), *** (PV<0.005).

FIGS. 14A-14G: Weighted matrixes of the transcription factor binding site sequences for the transcription factors (14A) SOX9, (14B) GATA10, (14C) TCF7L1, (14D-E) LEF1, (14F) MYBL2 and (14G) MYB.

### DETAILED DESCRIPTION OF THE INVENTION

The present invention, in some embodiments, provides Nucleic acid molecules configured to specifically express a sequence of interest in a cancer cell, comprising: a transcription factor binding region comprising 3-10 repeats of a transcription factor binding site, a minimal promoter and the sequence of interest operatively linked to the transcription factor binding region and minimal promoter, wherein the transcription factor is selected from SOX9, GATA10, TCF7L1, LEF1, MYBL2 and MYB are provided. Nucleic acid molecule systems and compositions comprising the nucleic acid molecules are also provided as are methods of treating cancer.

The present invention is based, at least in part, on the surprising discovery of specific transcription factor binding sites that can confer to an expression construct cancer cell specificity. Four transcription factors binding sites (TF-BS) sequences derived from (SOX9, GATA10, TCF7L1 and LEF1) were found to produce expression in colorectal cancer cells but not healthy colorectal cells or indeed healthy cells from other tissue. Two TF-BS derived from (MYBL2 and MYB) were found to produce expression in breast cancer cells but not healthy breast cells or indeed healthy cells from other tissue. Promoters containing repeats of the binding sites for these transcription factors were found to have a very restricted expression pattern with high expression in the target cancer cells and low or no expression in healthy cells. A dual promoter integrator (DPI) circuit that makes use of two promoters containing different transcription factor binding sites increased the specificity even more. The circuit showed no off-target expression of the output molecule (EGFP or immune-modulator protein/s) in healthy cells while retaining robust expression of the output molecule in the target cancer cells. Moreover, the use of the DPI circuits was sufficient to evoke a selective and effective immune response against the cancer.

By a first aspect, there is provided a nucleic acid molecule comprising a transcription factor binding region comprising repeats of a transcription factor (TF) binding site, a promoter and a sequence of interest, wherein the transcription factor is selected from SOX9, GATA10, TCF7L1, LEF1, MYBL2 and MYB.

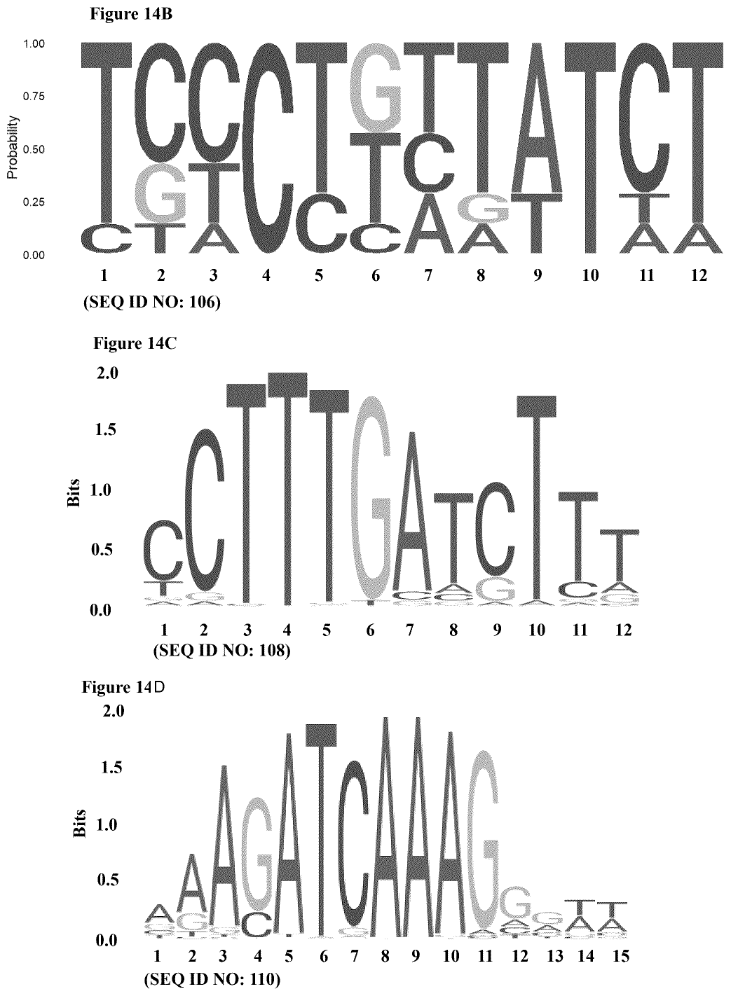

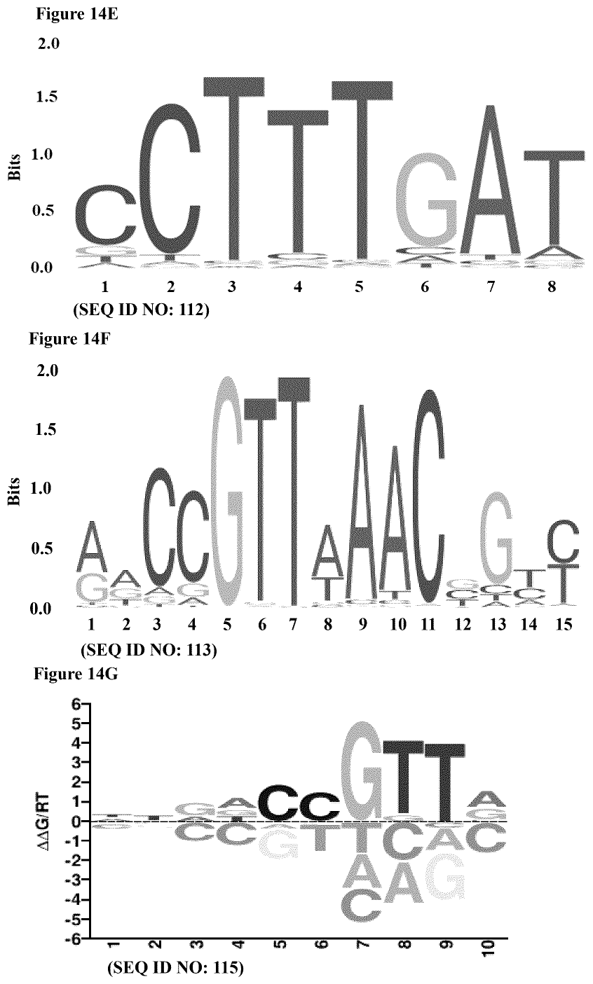

The consensus sequences for the SOX9, GATA10, TCF7L1, LEF1, MYBL2 and MYB binding sites are provided in FIGS. 14A, 14B, 14C, 14D-14E, 14F and 14G, respectively. It will be well understood to a skilled artisan that transcription factor binding sites are degenerative and so a consensus sequence more accurately depicts the binding site than a single nucleotide sequence. It will be understood that when a degenerate base is given (e.g., “S” referring to “G or C”) all possibilities for this base are being recited as well as all possible combinations with other degenerate bases. It will be understood that “N” refers to any base, i.e., “A”, “T”, “C” or “G”; “R” refers to “G” or “A”; “Y” refers to “C” or “T”; “K” refers to “G” or “T”; “M” refers to “A” or “C”; “S” refers to “G” or “C”; “W” refers to “A” or “T”; “B” refers to “G”, “T” or “C”; “D” refers to “G”, “A”, or “T”; “H” refers to “A”, “C” or “T” and “V” refers to “G”, “C” or “A”.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, lncRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides. In some embodiments, the nucleic acid molecule is a DNA molecule. In some embodiments, the DNA is double stranded DNA. In some embodiments, the DNA is single stranded DNA. In some embodiments, the nucleic acid molecule is RNA.

In some embodiments, the nucleic acid molecule is configured to express the sequence of interest in a cancer cell. In some embodiments, express is specifically express. In some embodiments, specific expression is expression that is greater in the cancer cell than a control cell. In some embodiments, specific expression comprises minimal expression in a control cell. In some embodiments, minimal is no expression. In some embodiments, no expression is no detectable expression. In some embodiments, detectable is by a reporter assay. Methods of detection are well known in the art and any such method may be used to detect expression from the molecules of the invention, including western blotting, ELISA, PCR, sequencing, immunostaining, FACS and the like. In some embodiments, specific expression comprises a ratio of expression in the cancer cell to a control cell of at least 2:1, 3:1, 5:1, 10:1, 20:1, 50:1, 100:1, 150:1, 200:1, 250:1, 300:1, 400:1, 500:1 or 1000:1. Each possibility represents a separate embodiment of the invention. In some embodiments, expression comprises expression that is greater than a predetermined threshold. In some embodiments, the threshold is the expression produced by an empty vector. In some embodiments, the threshold is an MFI of 9. In some embodiments, MFI is of a fluorescent reporter protein produced by the promoter. In some embodiments, the reporter protein is EGFP. In some embodiments, a strong promoter is a constitutive promoter. In some embodiments, the threshold is 10% of the expression produced by a strong promoter. In some embodiments, a strong promoter is a constitutive promoter. In some embodiments, the constitutive promoter is the ubiquitin C (UBC) promoter. In some embodiments, the UBC promoter comprises ggcctccgcgccgggttttggcgcctcccgcgggcgcccccctcctcacggcgagcgctgccacgtcagacgaagggcgcag cgagcgtcctgatccttccgcccggacgctcaggacagcggcccgctgctcataagactcggccttagaaccccagtatcagca gaaggacattttaggacgggacttgggtgactctagggcactggttttctttccagagagcggaacaggcgaggaaaagtagtcc cttctcggcgattctgcggagggatctccgtggggcggtgaacgccgatgattatataaggacgcgccgggtgtggcacagctag ttccgtcgcagccgggatttgggtcgcggttcttgtttgtggatcgctgtgatcgtcacttggtgagtagcgggctgctgggctggcc ggggctttcgtggccgccgggccgctcggtgggacggaagcgtgtggagagaccgccaagggctgtagtctgggtccgcgag caaggttgccctgaactgggggttggggggagcgcagcaaaatggcggctgttcccgagtcttgaatggaagacgcttgtgagg cgggctgtgaggtcgttgaaacaaggtggggggcatggtgggcggcaagaacccaaggtcttgaggccttcgctaatgcggga aagctcttattcgggtgagatgggctggggcaccatctggggaccctgacgtgaagtttgtcactgactggagaactcggtttgtcg tctgttgcgggggcggcagttatggcggtgccgttgggcagtgcacccgtacctttgggagcgcgcgccctcgtcgtgtcgtgac gtcacccgttctgttggcttataatgcagggtggggccacctgccggtaggtgtgcggtaggcttttctccgtcgcaggacgcagg gttcgggcctagggtaggctctcctgaatcgacaggcgccggacctctggtgaggggagggataagtgaggcgtcagtttctttg gtcggttttatgtacctatcttcttaagtagctgaagctccggttttgaactatgcgctcggggttggcgagtgtgttttgtgaagtttttta ggcaccttttgaaatgtaatcatttgggtcaatatgtaattttcagtgttagactagtaaattgtccgctaaattctggccgtttttggcttttt tgttagac (SEQ ID NO: 32). In some embodiments, the UBC promoter consists of SEQ ID NO: 32. In some embodiments, the UBC promoter is used as the maximum expression and the empty vector is used as the minimum expression and the threshold for positive expression is an MFI 9. In some embodiments, MFI is a measure of relative expression. In some embodiments, the UBC promoter is used as the maximum expression and the empty vector is used as the minimum expression and the threshold for positive expression is an MFI 10. It will be understood by a skilled artisan that there are many ways of determining expression and thereby if expression in a given cell type is specific. The method provided herein is but one method for making such a determination and establishing a threshold for what is considered positive expression, however, any method known in the art may be used.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, expression is translation. In some embodiments, expression is transcription. Expression of a gene within a cell is well known to one skilled in the art. It can be carried out by, among many methods, transfection, viral infection, or direct alteration of the cell's genome.

In some embodiments, the nucleic acid molecule is a vector. In some embodiments, the vector is an expression vector. In some embodiments, the vector is configured to express in mammalian cells. In some embodiments, the mammal is humans. In some embodiments, the vector is configured to express in cancer cells. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector.

In some embodiments, the vector is introduced into the cell by standard methods including electroporation (e.g., as described in From et al., Proc. Natl. Acad. Sci. USA 82, 5824 (1985)), Heat shock, calcium phosphate, liposomes, infection by viral vectors, high velocity ballistic penetration by small particles with the nucleic acid either within the matrix of small beads or particles, or on the surface (Klein et al., Nature 327. 70-73 (1987)), and/or the like.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide. A person with skill in the art will appreciate that a protein of interest or output protein can also be expressed from a nucleic acid construct or system administered to the individual employing any suitable mode of administration, described hereinbelow (i.e., in vivo gene therapy).

In some embodiments, the nucleic acid molecule comprises a transcription factor binding region. In some embodiments, the binding region comprises the repeats of the TF binding site. In some embodiments, the repeats are at least 2 instances of the transcription factor binding site. In some embodiments, 2 instances are 2 copies. In some embodiments, the repeats are at least 2, 3, 4, 5, 6, 7, 8, 9 or 10 instances of the TF binding site. Each possibility represents a separate embodiment of the invention. In some embodiments, the repeats are at least 3 instances of the TF binding site. In some embodiments, the repeats are at least 4 instances of the TF binding site. In some embodiments, the repeats are at least 5 instances of the TF binding site. In some embodiments, the repeats are at least 6 instances of the TF binding site. In some embodiments, the repeats are 2-15, 2-12, 2-10, 2-9, 2-8, 2-7, 2-6, 2-5, 3-15, 3-12, 3-10, 3-9, 3-8, 3-7, 3-6, 3-5, 5-15, 5-12, 5-10, 5-9, 5-8, 5-7, 6-15, 6-12, 6-10, 6-9, 6-8 or 6-7 instances of the TF binding site. Each possibility represents a separate embodiment of the invention. It will be understood that each repeat refers to an instance of the binding site and the terms “repeats” and “instances” are used herein interchangeably. In some embodiments, the repeats are 3-10 instances. In some embodiments, the repeats are 5-10 instances. In some embodiments, the repeats are 5-9 instances. In some embodiments, the repeats are 6-9 instances.

In some embodiments, the repeats are binding sites of the same transcription factor. In some embodiments, the repeats are binding sites of different TFs. In some embodiments, the repeats comprise binding sites for at least 2, 3, or 4 different TFs. Each possibility represents a separate embodiment of the invention. In some embodiments, each repeat or instance is the same. In some embodiments, the same is the same sequence. In some embodiments, the repeats comprise at least 2 different binding sites for the TF. In some embodiments, not all repeats are the same. In some embodiments, all repeats are the same. In some embodiments, the repeats are all identical.

In some embodiments, the SOX9 binding site comprises NAACAAWRG (SEQ ID NO: 104). In some embodiments, the SOX9 binding site comprises the reverse complement of SEQ ID NO: 104. In some embodiments, the SOX9 binding site comprises SEQ ID NO: 104 and/or its reverse complement. In some embodiments, the SOX9 binding site comprises NAACAAWGG (SEQ ID NO: 105). In some embodiments, the SOX9 binding site comprises the reverse complement of SEQ ID NO: 105. In some embodiments, the SOX9 binding site comprises SEQ ID NO: 105 and/or its reverse complement. In some embodiments, the SOX9 binding site comprises GAACAAAGGCCCCTTT (SEQ ID NO: 1). In some embodiments, the SOX9 binding site comprises the reverse complement of SEQ ID NO: 1. In some embodiments, the SOX9 binding site comprises SEQ ID NO: 1 and/or its reverse complement. In some embodiments, the SOX9 binding site consists of SEQ ID NO: 1 and/or its reverse complement. In some embodiments, the TF is SOX9 and the nucleic acid molecule is configured to specifically express the sequence of interest in a colorectal cancer cell. In some embodiments, the TF is SOX9 and the nucleic acid molecule is configured to specifically express the sequence of interest in a breast cancer cell.

In some embodiments, the GATA10 binding site comprises YBHCYBHDWTHW (SEQ ID NO: 106). In some embodiments, the GATA10 binding site comprises the reverse complement of SEQ ID NO: 106. In some embodiments, the GATA10 binding site comprises SEQ ID NO: 106 and/or its reverse complement. In some embodiments, the GATA10 binding site comprises TBHCTBHDATHT (SEQ ID NO: 107). In some embodiments, the GATA10 binding site comprises the reverse complement of SEQ ID NO: 107. In some embodiments, the GATA10 binding site comprises SEQ ID NO: 107 and/or its reverse complement. In some embodiments, the GATA10 binding site comprises TCCCTTTTATCT (SEQ ID NO: 2). In some embodiments, the GATA10 binding site comprises the reverse complement of SEQ ID NO: 2. In some embodiments, the GATA10 binding site comprises SEQ ID NO: 2 and/or its reverse complement. In some embodiments, the GATA10 binding site consists of SEQ ID NO: 2 and/or its reverse complement. In some embodiments, the TF is GATA10 and the nucleic acid molecule is configured to specifically express the sequence of interest in a colorectal cancer cell.

In some embodiments, the TCF7L1 binding site comprises YCTTTGATSTY (SEQ ID NO: 108). In some embodiments, the TCF7L1 binding site comprises the reverse complement of SEQ ID NO: 108. In some embodiments, the TCF7L1 binding site comprises SEQ ID NO: 108 and/or its reverse complement. In some embodiments, the TCF7L1 binding site comprises CCTTTGATSTT (SEQ ID NO: 109). In some embodiments, the TCF7L1 binding site comprises the reverse complement of SEQ ID NO: 109. In some embodiments, the TCF7L1 binding site comprises SEQ ID NO: 109 and/or its reverse complement. In some embodiments, the TCF7L1 binding site comprises TGCCTTTGATGTT (SEQ ID NO: 3). In some embodiments, the TCF7L1 binding site comprises the reverse complement of SEQ ID NO: 3. In some embodiments, the TCF7L1 binding site comprises SEQ ID NO: 3 and/or its reverse complement. In some embodiments, the TCF7L1 binding site consists of SEQ ID NO: 3 and/or its reverse complement. In some embodiments, the TF is TCF7L1 and the nucleic acid molecule is configured to specifically express the sequence of interest in a colorectal cancer cell.

In some embodiments, the LEF1 binding site comprises NNASATCAAAGNNNN (SEQ ID NO: 110). In some embodiments, the LEF1 binding site comprises the reverse complement of SEQ ID NO: 110. In some embodiments, the LEF1 binding site comprises SEQ ID NO: 110 and/or its reverse complement. In some embodiments, the LEF1 binding site comprises NNRASATCAAAGNNWW (SEQ ID NO: 111). In some embodiments, the LEF1 binding site comprises the reverse complement of SEQ ID NO: 111. In some embodiments, the LEF1 binding site comprises SEQ ID NO: 111 and/or its reverse complement. In some embodiments, the LEF1 binding site comprises AAAGATCAAAGGGTT (SEQ ID NO: 4). In some embodiments, the LEF1 binding site comprises the reverse complement of SEQ ID NO: 4. In some embodiments, the LEF1 binding site comprises SEQ ID NO: 4 and/or its reverse complement. In some embodiments, the LEF1 binding site consists of SEQ ID NO: 4 and/or its reverse complement. In some embodiments, the LEF1 binding site comprises CCTTTGAT (SEQ ID NO: 112). In some embodiments, the LEF1 binding site comprises the reverse complement of SEQ ID NO: 112. In some embodiments, the LEF1 binding site comprises SEQ ID NO: 112 and/or its reverse complement. In some embodiments, the LEF1 binding site comprises TCCTTTGATTTGCT (SEQ ID NO: 5). In some embodiments, the LEF1 binding site comprises the reverse complement of SEQ ID NO: 5. In some embodiments, the LEF1 binding site comprises SEQ ID NO: 5 and/or its reverse complement. In some embodiments, the LEF1 binding site consists of SEQ ID NO: 5 and/or its reverse complement. In some embodiments, the TF is LEF1 and the nucleic acid molecule is configured to specifically express the sequence of interest in a colorectal cancer cell.

In some embodiments, the MYBL2 binding site comprises RACSGTTAACSKHY (SEQ ID NO: 113). In some embodiments, the MYBL2 binding site comprises the reverse complement of SEQ ID NO: 113. In some embodiments, the MYBL2 binding site comprises SEQ ID NO: 113 and/or its reverse complement. In some embodiments, the MYBL2 binding site comprises AACSGTTAACSGHY (SEQ ID NO: 114). In some embodiments, the MYBL2 binding site comprises the reverse complement of SEQ ID NO: 114. In some embodiments, the MYBL2 binding site comprises SEQ ID NO: 114 and/or its reverse complement. In some embodiments, the MYBL2 binding site comprises AACGGTTAACGGTT (SEQ ID NO: 6). In some embodiments, the MYBL2 binding site comprises the reverse complement of SEQ ID NO: 6. In some embodiments, the MYBL2 binding site comprises SEQ ID NO: 6 and/or its reverse complement. In some embodiments, the MYBL2 binding site consists of SEQ ID NO: 6 and/or its reverse complement. In some embodiments, the TF is MYBL2 and the nucleic acid molecule is configured to specifically express the sequence of interest in a breast cancer cell.

In some embodiments, the MYB binding site comprises NNDDCCGTTD (SEQ ID NO: 115). In some embodiments, the MYB binding site comprises the reverse complement of SEQ ID NO: 115. In some embodiments, the MYB binding site comprises SEQ ID NO: 115 and/or its reverse complement. In some embodiments, the MYB binding site comprises TTGACCGTTA (SEQ ID NO: 7). In some embodiments, the MYB binding site comprises the reverse complement of SEQ ID NO: 7. In some embodiments, the MYB binding site comprises SEQ ID NO: 7 and/or its reverse complement. In some embodiments, the MYB binding site consists of SEQ ID NO: 7 and/or its reverse complement. In some embodiments, the TF is MYB and the nucleic acid molecule is configured to specifically express the sequence of interest in a breast cancer cell.

In some embodiments, the nucleic acid molecule is configured to specifically express the sequence of interest in a target cell. In some embodiments, the target cell is a cancer cell. In some embodiments, the target cell is a malignant cell. In some embodiments, the target cell is a disease cell. In some embodiments, the cancer is colorectal cancer. In some embodiments, the cancer is breast cancer. In some embodiments, the colorectal cancer is a colorectal adenocarcinoma. In some embodiments, the colorectal cancer is a colorectal carcinoma. In some embodiments, the cancer is a primary cancer. In some embodiments, the cancer is a metastasis of the cancer. In some embodiments, colorectal cancer is a cancer from the large intestine. In some embodiments, the cancer is a lung metastasis of colorectal cancer. In some embodiments, the cancer is an epithelial cancer. In some embodiments, the cancer is a breast adenocarcinoma. In some embodiments, the cancer is a breast carcinoma. In some embodiments, the breast cancer is primary ductal carcinoma. In some embodiments, the cancer is metastatic.

In some embodiments, the nucleic acid molecule further comprises a spacer between TF binding site repeats. In some embodiments, the TF binding region comprises a spacer between repeats. In some embodiments, there is a spacer between each repeat. In some embodiments, each repeat is separated from an adjacent repeat by a spacer. In some embodiments, the 5′ repeat is proceeded by a spacer. In some embodiments, the 3′ repeat is followed by a spacer. In some embodiments, each spacer is the same. In some embodiments, each spacer is different. In some embodiments, a spacer is between 1-20, 1-15, 1-10, 1-7, 1-5, 1-3, 2-20, 2-15, 2-10, 2-7, 2-5, 2-3, 3-20, 3-15, 3-10, 3-7 or 3-5 nucleotides in length. Each possibility represents a separate embodiment of the invention. In some embodiments, the spacer is 1-5 nucleotides in length. In some embodiments, the spacer consists of 1-5 nucleotides. In some embodiments, the spacer comprises 3 nucleotides. In some embodiments, the spacer consists of 3 nucleotides. In some embodiments, the spacer is 3 nucleotides in length. It will be understood that the sequence of the spacer is not important but rather that there be sufficient distance between each binding site such that a TF can bind at each binding site. If the binding sites are too close, then the binding of a first TF to a first binding site may occlude other of the other binding sites. Thus, it may be that for larger TFs a longer spacer may be needed and for smaller TFs a smaller spacer may be used. Examples of 3-mers that can be used as spacers include, but are not limited to ATC, GCT, TGA, CAG, ATG and the like.

In some embodiments, the TF binding region comprises at least 50, 60, 70, 75, 80, 90, 95, 100, 105, or 110 nucleotides. Each possibility represents a separate embodiment of the invention. In some embodiments, the TF binding region comprises at least 50 nucleotides. In some embodiments, the TF binding region comprises at least 100 nucleotides. In some embodiments, the TF binding region comprises at least 110 nucleotides. In some embodiments, the TF binding region comprises at least 111 nucleotides. In some embodiments, the TF binding region comprises at most 125, 130, 135, 140, 145, 150, 160, 170, 175, 180, 190, 200, 210, 220, 225, 230, 240, or 250 nucleotides. Each possibility represents a separate embodiment of the invention. In some embodiments, the TF binding region comprises at most 125 nucleotides. Each possibility represents a separate embodiment of the invention. In some embodiments, the TF binding region comprises at most 190 nucleotides. In some embodiments, the TF binding region comprises at most 200 nucleotides. In some embodiments, the TF binding region comprises at most 250 nucleotides. In some embodiments, the TF binding region consists of between 50 and 250 nucleotides. In some embodiments, the TF binding region consists of between 100 and 150 nucleotides. In some embodiments, the TF binding region consists of between 110 and 125 nucleotides. In some embodiments, the TF binding region consists of between 111 and 125 nucleotides.

In some embodiments, the TF binding region comprises 6 repeats of a SOX9 binding site. In some embodiments, the TF binding region comprises 6 repeats of SEQ ID NO: 1. In some embodiments, each repeat of SOX9 is separated by a 3-nucleotide spacer. In some embodiments, the TF binding region comprises GAACAAAGGCCCCTTTatcGAACAAAGGCCCCTTTgctGAACAAAGGCCCCTTTtga GAACAAAGGCCCCTTTcagGAACAAAGGCCCCTTTatgGAACAAAGGCCCCTTT (SEQ ID NO: 8). In some embodiments, the TF binding region consists of SEQ ID NO: 8.

In some embodiments, the TF binding region comprises 8 repeats of a GATA10 binding site. In some embodiments, the TF binding region comprises 8 repeats of SEQ ID NO: 2. In some embodiments, each repeat of GATA10 is separated by a 3-nucleotide spacer. In some embodiments, the TF binding region comprises TCCCTTTTATCTatcTCCCTTTTATCTgctTCCCTTTTATCTtgaTCCCTTTTATCTcagT CCCTTTTATCTatgTCCCTTTTATCTcgtTCCCTTTTATCTtacTCCCTTTTATCT (SEQ ID NO: 9). In some embodiments, the TF binding region consists of SEQ ID NO: 9.

In some embodiments, the TF binding region comprises 8 repeats of a TCF7L1 binding site. In some embodiments, the TF binding region comprises 8 repeats of SEQ ID NO: 3. In some embodiments, each repeat of TCF7L1 is separated by a 3-nucleotide spacer. In some embodiments, the TF binding region comprises TGCCTTTGATGTTatcTGCCTTTGATGTTgctTGCCTTTGATGTTtgaTGCCTTTGATG TTcagTGCCTTTGATGTTatgTGCCTTTGATGTTcgtTGCCTTTGATGTTtacTGCCTTT GATGTT (SEQ ID NO: 10). In some embodiments, the TF binding region consists of SEQ ID NO: 10.

In some embodiments, the TF binding region comprises 7 repeats of a LEF1 binding site. In some embodiments, the TF binding region comprises 7 repeats of SEQ ID NO: 4. In some embodiments, the TF binding region comprises 7 repeats of SEQ ID NO: 5. In some embodiments, each repeat of LEF1 is separated by a 3-nucleotide spacer. In some embodiments, the TF binding region comprises AAAGATCAAAGGGTTatcAAAGATCAAAGGGTTgctAAAGATCAAAGGGTTtgaAA AGATCAAAGGGTTcagAAAGATCAAAGGGTTatgAAAGATCAAAGGGTTcgtAAAG ATCAAAGGGTT (SEQ ID NO: 11). In some embodiments, the TF binding region consists of SEQ ID NO: 11. In some embodiments, the TF binding region comprises TCCTTTGATTTGCTatcTCCTTTGATTTGCTgctTCCTTTGATTTGCTtgaTCCTTTGAT TTGCTcagTCCTTTGATTTGCTatgTCCTTTGATTTGCTcgtTCCTTTGATTTGCT (SEQ ID NO: 12). In some embodiments, the TF binding region consists of SEQ ID NO: 12.

In some embodiments, the TF binding region comprises 7 repeats of a MYBL2 binding site. In some embodiments, the TF binding region comprises 7 repeats of SEQ ID NO: 6. In some embodiments, each repeat of MYBL2 is separated by a 3-nucleotide spacer. In some embodiments, the TF binding region comprises AACGGTTAACGGTTatcAACGGTTAACGGTTgctAACGGTTAACGGTTtgaAACGGT TAACGGTTcagAACGGTTAACGGTTatgAACGGTTAACGGTTcgtAACGGTTAACG GTT (SEQ ID NO: 13). In some embodiments, the TF binding region consists of SEQ ID NO: 13.

In some embodiments, the TF binding region comprises 9 repeats of a MYB binding site. In some embodiments, the TF binding region comprises 9 repeats of SEQ ID NO: 7. In some embodiments, each repeat of MYB is separated by a 3-nucleotide spacer. In some embodiments, the TF binding region comprises TTGACCGTTAatcTTGACCGTTAgctTTGACCGTTAtgaTTGACCGTTAcagTTGACCG TTAatgTTGACCGTTAcgtTTGACCGTTAtacTTGACCGTTAgaaTTGACCGTTA (SEQ ID NO: 14). In some embodiments, the TF binding region consists of SEQ ID NO: 14.

In some embodiments, the TF binding region comprises any one of SEQ ID NO: 8-14. In some embodiments, the TF binding region consists of any one of SEQ ID NO: 8-14. In some embodiments, the TF binding region comprises or consists of any one of SEQ ID NO: 8-14 or any sequence equivalent to SEQ ID NO: 8-14 in which a transcription factor binding site is replaced with its reverse complement. It will be understood that an equivalent sequence comprises no other changes than the replacement of at least one TF binding site with its reverse complement. In some embodiments, 1, 2, 3, 4, 5, 6, 7, or 8 of the TF binding sites are replaced with their reverse complements. Each possibility represents a separate embodiment of the invention.

In some embodiments, the nucleic acid molecule comprises a promoter. In some embodiments, the promoter is a minimal promoter. In some embodiments, the promoter is a constitutively active promoter. In some embodiments, the promoter is not activated unless a transcription factor binds to said TF binding site. In some embodiments, the promoter is not activated unless a transcription factor binds to said binding region. In some embodiments, binding of a TF to the TF binding site or binding region activates the promoter. In some embodiments, the TF binding site repeats are operatively linked to the promoter. In some embodiments, the TF binding region is operatively linked to the promoter. In some embodiments, the TF binding site repeats are 5′ to the promoter. In some embodiments, the TF binding region is 5′ to the promoter. In some embodiments, the TF binding site repeats are 3′ to the promoter. In some embodiments, the TF binding region is 3′ to the promoter. In some embodiments, the TF binding site repeats are within the promoter. In some embodiments, the TF binding region is within the promoter. In some embodiments, the TF binding region or the TF binding sites are a regulatory element regulating the promoter. In some embodiments, regulating is activating. In some embodiments, the promoter in absence of TF binding to a binding site does not transcribe the sequence of interest. In some embodiments, does not transcribe is does not substantially transcribe. In some embodiments, does not transcribe is does not detectably transcribe. In some embodiments, does not transcribe is does not significantly transcribe. In some embodiments, does not transcribe is does not transcribe at levels above a predetermined threshold. In some embodiments, the predetermined threshold is background levels. In some embodiments, the predetermined threshold is zero. Examples of minimal promoters which can be used as the promoter within the nucleic acid molecule of the invention can be found in Ede et al., “Quantitative Analyses of Core Promoters Enable Precise Engineering of Regulated Gene Expression in Mammalian Cells”, ACS Synth. Biol., 2016, 5, 5, 395-404, herein incorporated by reference in its entirety.

In some embodiments, the promoter is a viral promoter. In some embodiments, the promoter is an adenoviral promoter. In some embodiments, the promoter is the late adenoviral promoter. In some embodiments, the promoter is the adenoviral late promoter. In some embodiments, the adenoviral late promoter is the adenoviral major late promoter. In some embodiments, the minimal promoter is a fragment from a promoter. In some embodiments, the promoter is a core promoter. In some embodiments, the fragment is from −38 to +6 of the late adenoviral promoter. In some embodiments, a fragment from −38 to +6 of the late adenoviral promoter comprises ggggggctataaaagggggtgggggcgttcgtcctcactct (SEQ ID NO: 18). In some embodiments, a fragment from −38 to +6 of the late adenoviral promoter consists of SEQ ID NO: 18. In some embodiments, the promoter comprises SEQ ID NO: 18. In some embodiments, the promoter consists of SEQ ID NO: 18. In some embodiments, the promoter is the YB_TATA promoter. In some embodiments, the promoter is the YB_TATA core promoter. In some embodiments, the YB_TATA core promoter comprises tctagagggtatataatgggggcca (SEQ ID NO: 30). In some embodiments, the YB_TATA core promoter consists of SEQ ID NO: 30. In some embodiments, the promoter comprises SEQ ID NO: 30. In some embodiments, the promoter consists of SEQ ID NO: 30. In some embodiments, the promoter is selected from SEQ ID NO: 18 and SEQ ID NO: 30.

In some embodiments, the sequence of interest is operatively linked to the promoter. In some embodiments, the sequence of interest is operatively linked to the transcription factor binding site repeats. In some embodiments, the sequence of interest is operatively linked to the transcription factor binding region. In some embodiments, the sequence of interest is operatively linked to the promoter and the TF binding site repeats. In some embodiments, the sequence of interest is operatively linked to the promoter and the TF binding region. As used herein, the term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, being operably linked allows for transcription of the sequence of interest. In some embodiments, being operably linked allows for transcription of the sequence of interest upon binding of a TF to a TF repeat. In some embodiments, being operably linked allows for transcription of the sequence of interest upon binding of a TF to the TF binding region. In some embodiments, the promoter and TF repeats are 5′ to the sequence of interest. In some embodiments, the promoter and TF binding region are 5′ to the sequence of interest.

In some embodiments, the TF binding region and the promoter are separated by a spacer. In some embodiments, the most '3 TF binding site and the promoter are separated by a spacer. In some embodiments, the spacer comprises between 10-50, 10-40, 10-30, 10-20, 15-50, 15-40, 15-30, 15-20, 18-50, 10-40, 18-30, or 18-20 nucleotides. Each possibility represents a separate embodiment of the invention. In some embodiments, the spacer comprises between 10-20 nucleotides. In some embodiments, the spacer comprises at least 5, 8, 10, 12, 15, 17, 18, 20, 22 or 25 nucleotides. Each possibility represents a separate embodiment of the invention. In some embodiments, the spacer comprises at least 10 nucleotides. In some embodiments, the spacer comprises about 18 nucleotides.

In some embodiments, the nucleic acid molecule is configured to express the sequence of interest in a cancer cell. In some embodiments, the promoter and TF binding sites/binding region are configured to express the sequence of interest in a cancer cell. In some embodiments, the promoter and TF binding sites/binding region drive expression of the sequence of interest in a cancer cell. In some embodiments, expression is mRNA expression. In some embodiments, expression is protein expression. In some embodiments, expression comprises transcription. In some embodiments, expression comprises translation.

The term “expression” as used herein refers to the biosynthesis of a gene product, including the transcription and/or translation of that gene product. Thus, expression of a nucleic acid molecule may refer to transcription of the nucleic acid fragment (e.g., transcription resulting in mRNA or other functional RNA) and/or translation of RNA into a precursor or mature protein (polypeptide). Expressing of a gene within a cell is well known to one skilled in the art. It can be carried out by, among many methods, transfection, viral infection, or direct alteration of the cell's genome.

In some embodiments, the expression is specific expression. In some embodiments, specific is cancer specific. In some embodiments, specific is breast cancer specific. In some embodiments, specific is colorectal cancer specific. In some embodiments, specific is breast cancer and/or colorectal cancer specific. In some embodiments, specific expression comprises of target expression that is at or below a predetermined threshold. In some embodiments, off-target expression is expression in non-cancerous cells. In some embodiments, the non-cancerous cells are of the same cell type as the cancer. In some embodiments, off-target expression is expression in non-cancerous breast cells. In some embodiments, off-target expression is expression in non-cancerous colorectal cells. In some embodiments, the threshold is any expression. In some embodiments, the threshold is expression from an empty vector. In some embodiments, the threshold is zero expression. In some embodiments, the threshold is above MFI 9. In some embodiments, the threshold is MFI 10. In some embodiments, 9 is 9 of expression driven by a strong promoter. In some embodiments, 10 is 10 of expression driven by a strong promoter. In some embodiments, the strong promoter is the UBC promoter. In some embodiments, specific expression comprises no off-target expression. In some embodiments, no expression is no detectable expression. In some embodiments, no expression is no significant expression. Methods of determining expression or measuring expression are well known in the art and any such method may be used. Examples include, but are not limited to, a reporter assay such as is described herein below, RT-PCR, sequencing, western blot, and immunoassay.

In some embodiments, the nucleic acid molecule further comprises an untranslated region (UTR). In some embodiments, the nucleic acid molecule comprises a 5′ UTR. In some embodiments, the nucleic acid molecule comprises a 3′ UTR. In some embodiments, the 5′ UTR is between the promoter and the sequence of interest. In some embodiments, the 5′ UTR is adjacent to the 3′ end of the promoter. In some embodiments, the 5′ UTR is directly adjacent to the promoter. In some embodiments, the 5′ UTR comprises agatctgcgatctaagtaagcttggcattccggtactgttggtaaa (SEQ ID NO: 31). In some embodiments, the 5′ UTR consists of SEQ ID NO: 31.

In some embodiments, the sequence of interest is an open reading frame. As used herein, the term “open reading frame” refers to a nucleic acid sequence that can be translated into a protein. In some embodiments, the sequence of interest is a coding region. In some embodiments, the open reading frame comprises a start codon. In some embodiments, the start codon is a methionine codon. In some embodiments, the open reading frame comprises a stop codon. In some embodiments, the open reading frame comprises only one stop codon. In some embodiments, the open reading frame comprises a stop codon at its 3′ terminus. In some embodiments, the open reading frame has been codon optimized. In some embodiments, codon optimized is optimized for expression in a target cell. In some embodiments, the target cell is a mammalian cell. In some embodiments, the open reading frame encoding the first fusion protein is codon optimized. In some embodiments, the open reading frame encoding the second fusion protein is codon optimized. In some embodiments, a portion of the open reading frame encoding a protein binding domain is codon optimized.

In some embodiments, the sequence of interest encodes a protein. In some embodiments, the protein is a fusion protein. In some embodiments, the protein is an input of an AND gate circuit. In some embodiments, the sequence of interest encodes a therapeutic molecule. In some embodiments, the therapeutic molecule is configured to treat cancer. In some embodiments, the therapeutic molecule is an anticancer molecule. In some embodiments, the therapeutic protein is proapoptotic protein. In some embodiments, the therapeutic protein is a toxin.

By another aspect, there is provided a nucleic acid molecule system comprising at least two nucleic acid molecules of the invention, wherein a first nucleic acid molecule and a second nucleic acid molecule comprise repeats transcription factor binding sites of different transcription factors.

In some embodiments, the system comprises a first nucleic acid molecule of the invention, wherein the sequence of interest encodes a first fusion protein. In some embodiments, the system comprises a second nucleic acid molecule of the invention, wherein the sequence of interest encodes a second fusion protein. In some embodiments, the first fusion protein comprises a DNA binding domain and a first protein binding domain. In some embodiments, the second fusion protein comprises a transcriptional activation domain and a second protein binding domain. In some embodiments, the first and second protein binding domains are dimerization domains. In some embodiments, the first protein binding domain and the second protein binding domain are configured to bind to each other. In some embodiments, dimerization is heterodimerization. In some embodiments, the first and second protein binding domains do not bind to themselves. In some embodiments, the first and second fusion proteins are configured to binding to each other. In some embodiments, the first nucleic acid molecule comprises TF-binding sites of a first TF and the second nucleic acid molecule comprises TF-binding sites of a second TF and wherein the first and second TFs are different TFs.

In some embodiments, the system comprises a third nucleic acid molecule. In some embodiments, the third nucleic acid molecule is the output construct. In some embodiments, the third nucleic acid molecule comprises a polynucleotide operable linked to a transcriptional regulatory element. In some embodiments, the third nucleic acid molecule is a DNA molecule. In some embodiments, the DNA binding domain binds to the transcriptional regulatory element. In some embodiments, the transcriptional regulatory element is configured to be bound by the DNA binding domain. In some embodiments, the transcriptional regulatory element comprises at least one instance of a binding site of the DNA binding domain.

As used herein, the term “fusion protein” refers to a single polypeptide chain that contains domains or moieties from two distinct proteins that do not appear in a single polypeptide chain in nature. In some embodiments, the fusion protein is a chimeric protein. In some embodiments, the fusion protein is an artificial protein. In some embodiments, the fusion protein is not found in nature. The fusion protein may be formed by the joining of two or more peptides through a peptide bond formed between the amino-terminus of one peptide and the carboxyl-terminus of another peptide. The fusion protein may be expressed as a single polypeptide fusion protein from a nucleic acid sequence encoding the single contiguous conjugate. In some embodiments, fusion proteins are created through the joining of two or more genes that originally coded for separate proteins. Recombinant fusion proteins may be created artificially by recombinant DNA technology for use in biological research or therapeutics. “Chimeric” or “chimera” usually designate hybrid proteins made of polypeptides having different functions or physicochemical patterns. For example, a fusion protein can comprise a first part that is a DNA binding domain, and a second part (e.g., genetically fused to the first part) that comprises a protein binding domain. Methods of fusion protein generation, recombinant protein generation, recombinant DNA generation, and DNA fusion techniques are well known in the art, and any such method for making the chimeric molecules of the invention may be employed.

As used herein, the term “recombinant protein” refers to a protein which is coded for by a recombinant nucleic acid molecule (DNA or RNA) and is thus not naturally occurring. The term “recombinant DNA or RNA” refers to DNA or RNA molecules formed by laboratory methods of genetic recombination or rearrangement. Generally, this recombinant molecule is in the form of an mRNA, a vector, a plasmid or a virus, used to express the recombinant protein in a cell.

In some embodiments, the first fusion protein comprises a DNA binding domain. In some embodiments, the DNA binding domain is a targeting domain. In some embodiments, the first nucleic acid molecule is a targeting construct. In some embodiments, the DNA binding domain is a sequence specific DNA binding domain. DNA binding domains and their target sequences are well known in the art and any such DNA binding domain and target sequence may be used. Examples of these domains and targets include but are not limited to the Gal4-DNA binding domain and Gal4 binding site, the ZF1-DNA binding domain and the ZF1 binding site and the Tet-DNA binding domain and Tet binding site. In some embodiments, the DNA binding domain is a DNA binding domain of a transcription factor. In some embodiments, the domain is a part of a protein. In some embodiments, the domain is a fragment of a protein. In some embodiments, the domain is not the full protein. In some embodiments, the DNA binding domain is a native domain. In some embodiments, the DNA binding domain is an engineered domain.

In some embodiments, the DNA binding domain is the Gal4 DNA binding domain. In some embodiments, the Gal4 DNA binding domain is encoded by the nucleotide sequenceatgaagctactgtcttctatcgaacaagcatgcgatatttgccgacttaaaaagctcaagtgctccaaagaaaaaccga agtgcgccaagtgtctgaagaacaactgggagtgtcgctactctcccaaaaccaaaaggtctccgctgactagggcacatctgac agaagtggaatcaaggctagaaagactggaacagctatttctactgatttttcctcgagaagaccttgacatgattttgaaaatggatt ctttacaggatataaaagcattgttaacaggattatttgtacaagataatgtgaataaagatgccgtcacagatagattggcttcagtgg agactgatatgcctctaacattgagacagcatagaataagtgcgacatcatcatcggaagagagtagtaacaaaggtcaaagaca gttgactgtatcg (SEQ ID NO: 33). In some embodiments, the Gal4 DNA binding domain comprises the amino acid sequence MKLLSSIEQACDICRLKKLKCSKEKPKCAKCLKNNWECRYSPKTKRSPLTRAHLTE VESRLERLEQLFLLIFPREDLDMILKMDSLQDIKALLTGLFVQDNVNKDAVTDRLA SVETDMPLTLRQHRISATSSSEESSNKGQRQLTVS (SEQ ID NO: 34). In some embodiments, the Gal4 DNA binding domain consists of SEQ ID NO: 34. In some embodiments, the first nucleic acid molecule comprises a sequence of interest comprising SEQ ID NO: 33. In some embodiments, the first nucleic acid molecule comprises a sequence of interest that encodes SEQ ID NO: 33. In some embodiments, the Gal4 DNA binding domain comprises at least 85% identity to SEQ ID NO: 34 and comprises the ability to bind the Gal4 binding site. In some embodiments, the sequence encoding the Gal4 DNA binding domain comprises at least 85% identity to SEQ ID NO: 33 and encodes a protein with the ability to bind the Gal4 binding site. In some embodiments, the Gal4 binding site comprises CGGAGTACTGTCCTCCG (SEQ ID NO: 17). In some embodiments, the Gal4 binding site consists of SEQ ID NO: 17.

In some embodiments, the Gal4 binding domain comprises at least 1, 2, 3, 4, 5, 6, 7, or 8 Gal4 binding sites. Each possibility represents a separate embodiment of the invention. In some embodiments, the Gal4 binding domain comprises at least 3 Gal4 binding sites. In some embodiments, the Gal4 binding domain comprises at least 5 Gal4 binding sites. In some embodiments, the Gal4 binding domain comprises at least 8 Gal4 binding sites. In some embodiments, the Gal4 binding domain comprises 1, 2, 3, 4, 5, 6, 7, or 8 Gal4 binding sites. Each possibility represents a separate embodiment of the invention. In some embodiments, the Gal4 binding domain comprises 3 Gal4 binding sites. In some embodiments, the Gal4 binding domain comprises 5 Gal4 binding sites. In some embodiments, the Gal4 binding domain comprises 8 Gal4 binding sites.

In some embodiments, the DNA binding domain does not comprise transcription regulatory ability. In some embodiments, the first fusion protein does not comprise transcription regulatory ability. In some embodiments, regulatory ability is activation ability. In some embodiments, regulatory ability is inhibition ability. In some embodiments, the first fusion protein is not competent to activate transcription. It will be understood by a skilled artisan that the first and second fusion proteins are intended to bind together and thus reconstitute a complete transcriptional regulatory protein or complex. This ensures regulation of the output construct only in cells where both the first and second fusion proteins are expressed.

In some embodiments, the second fusion protein comprises a transcriptional regulatory element. In some embodiments, the second construct is a regulatory construct. In some embodiments, the second construct is an activation construct. In some embodiments, an element is a domain. In some embodiments, regulation is activation. In some embodiments, regulation is enhancement. In some embodiments, regulation is upregulation. In some embodiments, the element is a transcriptional activator. In some embodiments, the element is a transcriptional activator domain. In some embodiments, the element does not comprise DNA binding capability. In some embodiments, the second fusion protein does not comprise DNA binding capability. In some embodiments, the first fusion protein is the targeting module, and the second fusion protein is the activating module. In some embodiments, the first fusion protein and second fusion protein together reconstitute a transcription factor. Examples of transcriptional regulators include the Gal4 activation domain, the VP16 activation domain, the VP64 activation domain, the Tet activation domain and the lac suppressor, to name but a few. Any regulatory domain known in the art may be employed.

In some embodiments, the regulatory domain is the VP16 transcriptional activation domain. In some embodiments, the regulatory domain is the VP64 transcriptional activation domain. In some embodiments, the VP16 transcriptional activation domain is encoded by the nucleotide sequence atgtcgacggcccccccgaccgatgtcagcctgggggacgagctccacttagacggcgaggacgtggcgatggcgcatgccg acgcgctagacgatttcgatctggacatgttgggggacggggattccccgggtccggga (SEQ ID NO: 35). In some embodiments, the VP16 transcriptional activation domain comprises MSTAPPTDVSLGDELHLDGEDVAMAHADALDDFDLDMLGDGDSPGPG (SEQ ID NO: 36). In some embodiments, the VP16 transcriptional activation domain consists of SEQ ID NO: 36. In some embodiments, the second sequence of interest comprises SEQ ID NO: 35. In some embodiments, the second sequence of interest encodes SEQ ID NO: 36. Further, the regulation required, activation or repression, will be determined by the output produced by the system and its desired role in the target cell. It will be understood by a skilled artisan that the output of the system of the invention can be regulated by modifying the transcriptional regulatory element. This modification can be achieved by mutating the element, including multiple repeats of the element or by selecting an element with stronger or weaker activation ability. By selecting stronger activation output will go up, and conversely by selecting weaker activation will go down.

In some embodiments, the first fusion protein comprises a first protein binding domain. In some embodiments, the second fusion protein comprises a second protein binding domain. In some embodiments, the first and second protein binding domains are configured to bind to each other. In some embodiments, the first and second binding domains bind to each other. In some embodiments, the first and second protein binding domains are a first and second dimerization domains. In some embodiments, the first domain dimerizes with the second domain. Protein-protein binding domains are well known in the art and any such binding domain can be employed. In some embodiments, binding comprises forming a bond between the first protein binding domain and the second protein binding domain. In some embodiments, the bond is a covalent bond. In some embodiments, the bond is an electrostatic bond. In some embodiments, the bond is a chemical bond. In some embodiments, the bond is a disulfide bond. In some embodiments, the bond is a peptide bond. In some embodiments, the bond is an irreversible bond. In some embodiments, the bond is reversible. In some embodiments, the bond is a transient bond. In some embodiments, the bond is a permanent bond. In some embodiments, the bond is produced by protein splicing.

In some embodiments, protein binding domains are capable of binding with each other. In some embodiments, the first protein binding domain is capable of binding with the second protein binding domain. In some embodiments, the first and second protein binding domains are capable of binding with each other. In some embodiments, capable of binding is configured to bind. In some embodiments, binding is under physiological conditions. In some embodiments, binding is within a cell. In some embodiments, the cell is the target cell. In some embodiments, binding is within the cytoplasm. In some embodiments, binding is within a subject. In some embodiments, binding is in vivo. In some embodiments, binding is in vitro.

As used herein, the term “protein binding domain” refers to an amino acid sequence that upon contacting another amino acid sequence (the other protein binding domain) binds to it to form a stable bond. In some embodiments, the first and second protein binding domains are binding pair. Protein binding domains and dimerization domains are well known in the art, as many protein sequences are known to bind to each other. Examples of protein binding domains include the Coh2-DocS binding pair, the HrpS-HrpR binding pair, the antiparallel leucine zipper domains (Zip−/Zip+), the scFv anti-G4 and G4 binding pair (see Auslander et al., “Design of multipartite transcription factors for multiplexed logic genome integration control in mammalian cells”, ACS Synth. Biol., 2020 Nov. 20; 9(11): 2964-2970, herein incorporated by reference in its entirety), the hinge domain of antibody heavy chains, the CH1/CL domains of antibody heavy/light chains, and the ECD domains of TCR alpha/beta, to name but a few. Further, systems for finding binding partners, such as the SunTag system are well known in the art and can also yield binding pairs usable for the compositions and methods of the invention. In some embodiments, the first binding domain does not bind to itself. In some embodiments, the second binding domain does not bind to itself. In some embodiments, the binding domain is engineered to not bind to itself. In some embodiments, the first binding domain binds a plurality of second binding domains. In some embodiments, the second binding domain binds to a plurality of first binding domains.

In some embodiments, the first or second protein binding domain is Coh2. In some embodiments, the first or second protein binding domain comprises Coh2. In some embodiments, the first or second protein binding domain consists of Coh2. In some embodiments, Coh2 is encoded by gtggtagtagaaattggcaaagttacgggatctgttggaactacagttgaaatacctgtatatttcagaggagttccatccaaaggaa tagcaaactgcgactttgtgttcagatatgatccgaatgtattggaaattatagggatagatcccggagacataatagttgacccgaat cctaccaagagctttgatactgcaatatatcctgacagaaagataatagtattcctgtttgcggaagacagcggaacaggagcgtat gcaataactaaagacggagtatttgcaaaaataagagcaactgtaaaatcaagtgctccgggctatattactttcgacgaagtaggt ggatttgcagataatgacctggtagaacagaaggtatcatttatagacggtggtgttaacgtt (SEQ ID NO: 37). In some embodiments, SEQ ID NO: 37 further comprises a 5′ atg codon. In some embodiments, Coh2 comprises the amino acid sequence VVVEIGKVTGSVGTTVEIPVYFRGVPSKGIANCDFVFRYDPNVLEIIGIDPGDIIVDP NPTKSFDTAIYPDRKIIVFLFAEDSGTGAYAITKDGVFAKIRATVKSSAPGYITFDEV GGFADNDLVEQKVSFIDGGVNV (SEQ ID NO: 38). In some embodiments, Coh2 consists of SEQ ID NO: 38. In some embodiments, SEQ ID NO: 38 further comprises an N-terminal methionine residue. In some embodiments, Coh2 comprises at least 85% identity to SEQ ID NO: 38 and retains the ability to bind to DocS. In some embodiments, Coh2 is encoded by a sequence with at least 85% identity to SEQ ID NO: 37 and which encodes a protein that has the ability to bind to DocS.

In some embodiments, the first or second protein binding domain is DocS. In some embodiments, the first or second protein binding domain comprises DocS. In some embodiments, the first or second protein binding domain consists of DocS. In some embodiments, DocS is encoded by a nucleotide sequence comprising tctactaaattatacggcgacgtcaatgatgacggaaaagttaactcaactgacgctgtagcattgaagagatatgttttgagatcag gtataagcatcaacactgacaatgccgatttgaatgaagacggcagagttaattcaactgacttaggaattttgaagagatatattctc aaagaaatagatacattgccgtacaagaac (SEQ ID NO: 39). In some embodiments, the sequence that encodes DocS consists of SEQ ID NO: 39. In some embodiments, DocS comprises the amino acid sequence STKLYGDVNDDGKVNSTDAVALKRYVLRSGISINTDNADLNEDGRVNSTDLGILK RYILKEIDTLPYKN (SEQ ID NO: 40). In some embodiments, DocS consists of SEQ ID NO: 40. In some embodiments, DocS comprises a sequence with at least 85% identity to SEQ ID NO: 40 and retains the ability to bind to Coh2. In some embodiments, DocS is encoded by a sequence with at least 85% identity to SEQ ID NO: 39 and which encodes a protein with the ability to bind Coh2.

In some embodiments, the first and second binding domain are the Coh2 and DocS domains. It will be understood that it does not matter which molecule encodes the Coh2 and which DocS, or which fusion protein contains Coh2 and which DocS, all that matters is that the two are both present; one in one fusion protein (encoded by one sequence of interest) and one in the other fusion protein (encoded by the other sequence of interest). In some embodiments, Coh2 is the Coh2 protein. In some embodiments, Coh2 is a Coh2 domain. In some embodiments, DocS is the DocS protein. In some embodiments, DocS is a DocS domain.

In some embodiments, the first or second protein binding domain is HrpS. In some embodiments, the first or second protein binding domain comprises HrpS. In some embodiments, the first or second protein binding domain consists of HrpS. In some embodiments, HrpS is encoded by agtcttgatgaaaggtttgaggatgatctggacgaggagcgggttccgaatctggggatagttgccgaaagtatttcgcaactgggt atcgacgtgctgctatcgggtgagaccggcacgggcaaagacacgattgcccgacggattcatgagatgtcaggccgcaaagg gcgcctggtggcgatgaattgcgcggccattccggagtccctcgccgagagcgagttattcggcgtggtcagcggtgcctacacc ggcgctgatcgctccagagtcggttatgtcgaagcggcgcagggcggcacgctgtacctggatgagatcgatagcatgccgctg agcctgcaagccaaattgctgagggtgctggaaacccgagcgcttgaacggctgggttcgacgtcgacgatcaagctggatatct gcgtgatcgcctccgcccaatgctcgctggacgacgccgtcgagcgggggcagtttcgtcgcgatctgtattttcgcctgaacgtc ctgacactcaagcttcctccgctacgtaaccagtctgatcgcatagttcccctgttcacacgttttacggccgccgccgcgagggag ctcggtgttcccgttcccgatgtttgcccactgctgcacaaagtgctgctgggccacgactggcccggcaatatccgtgagctcaa ggcggcagccaaacgccatgtgctgggtttccccttgctgggcgccgagccgcagggcgaagagcacttggcctgtgggctca aatcgcaattgcgagtgatcgaaaaagccctgattcaggagtcgctcaagcgccacgacaattgtgtggattcggtaagcctggaa ctggacgtgccacgccgtacgctctatcgacgcatcaaagaattgcagatc (SEQ ID NO: 41). In some embodiments, SEQ ID NO: 41 further comprises a 5′ atg codon. In some embodiments, HrpS comprises the amino acid sequence SLDERFEDDLDEERVPNLGIVAESISQLGIDVLLSGETGTGKDTIARRIHEMSGRKG RLVAMNCAAIPESLAESELFGVVSGAYTGADRSRVGYVEAAQGGTLYLDEIDSMP LSLQAKLLRVLETRALERLGSTSTIKLDICVIASAQCSLDDAVERGQFRRDLYFRLN VLTLKLPPLRNQSDRIVPLFTRFTAAAARELGVPVPDVCPLLHKVLLGHDWPGNIR ELKAAAKRHVLGFPLLGAEPQGEEHLACGLKSQLRVIEKALIQESLKRHDNCVDS VSLELDVPRRTLYRRIKELQI (SEQ ID NO: 42). In some embodiments, HrpS consists of SEQ ID NO: 42. In some embodiments, SEQ ID NO: 42 further comprises an N-terminal methionine residue. In some embodiments, HrpS comprises at least 85% identity to SEQ ID NO: 42 and retains the ability to bind to HrpR. In some embodiments, HrpS is encoded by a sequence with at least 85% identity to SEQ ID NO: 41 and which encodes a protein that has the ability to bind to HrpR.

In some embodiments, the first or second protein binding domain is HrpR. In some embodiments, the first or second protein binding domain comprises HrpR. In some embodiments, the first or second protein binding domain consists of HrpR. In some embodiments, HrpR is encoded by a nucleotide sequence comprising agtacaggcatcgataaggacgtccgagagtgttggggcgtaactgcattatcagcgggtcatcaaattgcaatgaatagcgcgttt ctggatatggacttgctgttgtgcggggaaaccggcaccggcaaggacacactggccaaccgcattcacgagttgtccagcaggt cgggaccctttgtgggcatgaactgcgccgccattcccgagtcgctggcagagagccagttattcggtgtggtcaacggtgcattc accggcgtatgccgggctcgcgagggctacatagaggcctccagtggtggcaccttgtacctggatgaaatcgacagcatgccg ttgagcctgcaagccaaactgctgcgtgtgttggagagtcgaggtatcgagcgtctgggctcgaccgaatttatcccggtggatctg cggatcattgcctcggcccagcggccactggatgaactggtggaacaaggacttttccgtcgcgacctgttttttcggctcaacgtg ctgacgcttcacttgccagccttgcgcaaacgtcgtgaacagatcctgccattgttcgaccagttcacccagggtatcgctgccgag ttcggacgtcccgctcctgcgctggacagcgggcgtgtgcagctgctgctcagccacgactggccgggcaacatccgcgaattg aagtctgcggccaagcgcttcgtactcggcttccccttgctgggcgccgaccctgtggaagcgcttgaccctgccacggggctgc gcacgcaaatgcgcatcatcgagaaaatgctcatccaggatgccttgaagcggcacaggcacaatttcgacgcggtgcttcagga gttggagttgccaagacgcaccctgtatcaccgcatgaaggaactgggagttgcagcgccgatcgctgcgacggccggggtc (SEQ ID NO: 43). In some embodiments, the sequence that encodes HrpR consists of SEQ ID NO: 43. In some embodiments, HrpR comprises the amino acid sequence STGIDKDVRECWGVTALSAGHQIAMNSAFLDMDLLLCGETGTGKDTLANRIHELS SRSGPFVGMNCAAIPESLAESQLFGVVNGAFTGVCRAREGYIEASSGGTLYLDEID SMPLSLQAKLLRVLESRGIERLGSTEFIPVDLRIIASAQRPLDELVEQGLFRRDLFFR LNVLTLHLPALRKRREQILPLFDQFTQGIAAEFGRPAPALDSGRVQLLLSHDWPGNI RELKSAAKRFVLGFPLLGADPVEALDPATGLRTQMRIIEKMLIQDALKRHRHNFD AVLQELELPRRTLYHRMKELGVAAPIAATAGV (SEQ ID NO: 44). In some embodiments, HrpR consists of SEQ ID NO: 44. In some embodiments, HrpR comprises a sequence with at least 85% identity to SEQ ID NO: 44 and retains the ability to bind to HrpS. In some embodiments, HrpR is encoded by a sequence with at least 85% identity to SEQ ID NO: 43 and which encodes a protein with the ability to bind HrpS.

In some embodiments, the first and second binding domain are the HrpS and HrpR domains. It will be understood that it does not matter which molecule encodes the HrpS and which HrpR, or which fusion protein contains HrpS and which HrpR, all that matters is that the two are both present; one in one fusion protein (encoded by one sequence of interest) and one in the other fusion protein (encoded by the other sequence of interest). In some embodiments, HrpS is the HrpS protein. In some embodiments, HrpS is a HrpS domain. In some embodiments, HrpR is the HrpR protein. In some embodiments, HrpR is a HrpR domain.

In some embodiments, the system further comprises a regulating nucleic acid molecule. In some embodiments, the regulating nucleic acid molecule is a fourth nucleic acid molecule. In some embodiments, the regulating nucleic acid molecule comprises a coding region encoding an HrpV protein. In some embodiments, the HrpV is encoded by a sequence comprising atgatcgaggtgaccgagaagagcgccttctacgcccaagtggccgctcagagccctgctgtttggcctgtggctaatggcgtgg cctttgtgtccagaagggaacaccatgattggggcatcgccctgcacatcgagggaagagccctccgccccgaccagctgagag atgccctgcagagacggtttatggaaagcgaaagattcaaccactacttcctgttcctggatgtgcggcgggacttcgtggtctggc acgccgtgaacgagaagccaggcagctacgccagcctggacgacatcagaagacacgagctgatgctggccggcctggacca cctgtctgaggaaatgcac (SEQ ID NO: 61). In some embodiments, the HrpV is encoded by a sequence consisting of SEQ ID NO: 61. In some embodiments, the HrpV is encoded by a sequence with at least 85% identity to SEQ ID NO: 61 and encoding a protein that binds to HrpS. In some embodiments, the HrpV is encoded by a sequence with at least 85% identity to SEQ ID NO: 61 and encoding a protein that competes with HrpR for binding to HrpS. In some embodiments, HrpV comprises the amino acid sequence MIEVTEKSAFYAQVAAQSPAVWPVANGVAFVSRREHHDWGIALHIEGRALRPDQ LRDALQRRFMESERFNHYFLFLDVRRDFVVWHAVNEKPGSYASLDDIRRHELML AGLDHLSEEMH (SEQ ID NO: 62). In some embodiments, HrpV consists of SEQ ID NO: 62. In some embodiments, HrpV comprises a sequence with at least 85% identity to SEQ ID NO: 62 and retains the ability to bind HrpS. In some embodiments, HrpV comprises a sequence with at least 85% identity to SEQ ID NO: 62 and retains the ability to compete with HrpR for binding to HrpS.

In some embodiments, the regulating nucleic acid molecule comprises a promoter configured to express in non-cancerous cells. In some embodiments, non-cancerous cells are healthy cells. In some embodiments, express is specifically express. In some embodiments, the promoter does not express in cancer cells. In some embodiments, the coding region encoding HrpV is operatively linked to the promoter. A regulatory construct that expresses HrpV in non-cancerous cells produces an added layer of protection against off target expression of the polynucleotide. Even should the first and second molecules both produce expression in off target non-cancer cells the expression of HrpV will block heterodimerization between the first and second fusion proteins and thus inhibit expression of the polynucleotide.

In some embodiments, at least one of the first, and second protein binding domain is mutated to decrease binding to the other domain. In some embodiments, at least one of the first, and second protein binding domain is mutated to increase binding to the other domain. In some embodiments, both protein binding domains are mutated. In some embodiments, the mutated binding domain is DocS15. In some embodiments, the mutated binding domain is DocS102. It will be understood by a skilled artisan that the output of the system of the invention can be regulated by modifying the binding of the two protein binding domains. This modification can be achieved by mutating either or both binding domains, or by selecting a binding pair that binds each other with stronger or weaker affinity. By increasing affinity of binding output will go up, and conversely by decreasing affinity of binding output will go down.

In some embodiments, the DNA-binding domain is N-terminal to the protein binding domain. In some embodiments, the DNA-binding domain is C-terminal to the protein binding domain. In some embodiments, the protein binding domain is N-terminal to the DNA-binding domain. In some embodiments, the protein binding domain is C-terminal to the DNA-binding domain. In some embodiments, the DNA-binding domain and the protein binding domain are separated by a linker. In some embodiments, the transcriptional regulatory domain is N-terminal to the protein binding domain. In some embodiments, the transcriptional regulatory domain is C-terminal to the protein binding domain. In some embodiments, the protein binding domain is N-terminal to the transcriptional regulatory domain. In some embodiments, the protein binding domain is C-terminal to the transcriptional regulatory domain. In some embodiments, the transcriptional regulatory domain and the protein binding domain are separated by a linker.

In some embodiments, the sequence encoding the DNA-binding domain is 5′ to the sequence encoding the protein binding domain. In some embodiments, the sequence encoding the protein binding domain is 5′ to the sequence encoding the DNA-binding domain. In some embodiments, the sequence encoding the DNA-binding domain is 3′ to the sequence encoding the protein binding domain. In some embodiments, the sequence encoding the protein binding domain is 3′ to the sequence encoding the DNA-binding domain. In some embodiments, the sequence encoding the transcriptional activation domain is 5′ to the sequence encoding the protein binding domain. In some embodiments, the sequence encoding the protein binding domain is 5′ to the sequence encoding the transcriptional activation domain. In some embodiments, the sequence encoding the transcriptional activation domain is 3′ to the sequence encoding the protein binding domain. In some embodiments, the sequence encoding the protein binding domain is 3′ to the sequence encoding the transcriptional activation domain. In some embodiments, the domains are separated by a linker.

In some embodiments, the linker is a nucleotide linker. In some embodiments, the linker comprises multiples of 3 nucleotides such that the coding sequence downstream of the linker is in frame. In some embodiments, the linker is devoid of a stop codon. In some embodiments, the linker comprises 3, 6, 9, 12, 15, 18, 21, 24, 27, or 30 nucleotides. Each possibility represents a separate embodiment of the invention. In some embodiments, the linker comprises 6, 9, 12 or 15 nucleotides.

In some embodiments, the linker is a peptide linker. In some embodiments, a peptide linker is an amino acid linker. In some embodiments, the peptide linker comprises a peptide bond. In some embodiments, the peptide linker consists of a peptide bond. In some embodiments, the linker comprises at least 1, 2, 3, 4, 5, 6, 7, 8, 9 or 10 amino acids. Each possibility represents a separate embodiment of the invention. In some embodiments, the linker comprises at most 10, 15, 20, 25, 30, 40, 50, 60, 70, 75, 80, 90, 100, 150, 200, 250, 300, 250, 400, 450, 500, 550, 600, 650, 700, 750, 800, 850, 900, 950 or 1000 amino acids. Each possibility represents a separate embodiment of the invention. In some embodiments, the linker comprises 2-5 amino acids. In some embodiments, the linker is of a sufficient length to allow the DNA-binding domain to bind to the third transcriptional regulatory element and to allow the transcriptional regulatory element to regulate transcription of the polynucleotide.

In some embodiments, the targeting is to the third transcriptional regulatory element. In some embodiments, the DNA binding domain is configured to bind the third transcriptional regulatory element. In some embodiments, the DNA binding domain binds to the third transcriptional regulatory element. In some embodiments, the third transcriptional regulatory element comprises at least one binding site of the DNA binding domain. In some embodiments, the third transcriptional regulatory element comprises at least 1, 2, 3, 4, 5, 6, 7, 8, 9, 10, 12, 14, 15, 16, 18, 20, 25, 30, 35, 40, 45, 50, 60, 70, 80, 90 or 100 binding sites of the DNA binding domain. Each possibility represents a separate embodiment of the invention. In some embodiments, the third transcriptional regulatory element comprises 1, 2, 3, 4, 5, 6, 7, 8, 9, 10, 12, 14, 15, 16, 18, 20, 25, 30, 35, 40, 45, 50, 60, 70, 80, 90 or 100 binding sites of the DNA binding domain. Each possibility represents a separate embodiment of the invention. In some embodiments, the third transcriptional regulatory element comprises 3 binding sites of the DNA binding domain. In some embodiments, the third transcriptional regulatory element comprises at least 5 binding sites of the DNA binding domain. In some embodiments, the third transcriptional regulatory element comprises 8 binding sites of the DNA binding domain. In some embodiments, the third transcriptional regulatory element comprises at least 14 binding sites of the DNA binding domain. Repeated binding domains are known to increase binding efficiency and transcriptional regulation as they amplify the input to the regulatory element. It will be understood by a skilled artisan that the output of the system of the invention can be regulated by modifying the number of binding sites. By increasing the number of binding sites output will go up, and conversely by decreasing the number of binding sites output will go down.

In some embodiments, recruitment of the transcriptional activation domain to the transcriptional regulatory element drives transcription of the polynucleotide. In some embodiments, recruitment of the transcriptional activation domain to the transcriptional regulatory element in the third nucleic acid molecule drives transcription of the polynucleotide. It will be understood that the transcriptional activation domain will only be recruited to the transcriptional regulatory element of the third nucleic acid molecule is the first fusion protein and the second fusion protein have dimerized by the protein binding domains. When heterodimerization occurs the DNA binding domain can target the heterodimer complex to the transcriptional regulatory element of the third nucleic acid molecule and the transcriptional activation domain can then drive transcription of the polynucleotide. Thus, the polynucleotide is only transcribed when both the first fusion protein and the second fusion protein are produced and this will only occur if both promoters are activated. In this way the nucleic acid molecule system produces highly targeted expression of the polynucleotide. Both promoters controlled by their respective TF repeats must be active for the polynucleotide to be expressed. Thus, even if a first promoter/TF binding domain produces expression in target cells and some off target expression and a second promoter/TF binding domain also produced expression in target cells and some off target expression the polynucleotide will only be expressed in the target cells (or off target cells shared by the two promoter/TF binding domains). It will thus be clear that two TF binding regions should be selected that produce targeted expression in the same cancer cells and do not have overlapping off target expression. This will produce highly targeted expression only in the cancer cells.

In some embodiments, the binding site comprises a mutation that increased binding of the DNA binding domain. In some embodiments, the binding site comprises a mutation that decreased binding of the DNA binding domain. In some embodiments, the third regulatory element comprises both mutated and non-mutated binding sites. It will be understood by a skilled artisan that the output of the system of the invention can be regulated by modifying the binding site for the DNA binding domain. This modification can be achieved by mutating the binding site, or by selecting a binding domain and binding site with stronger or weaker affinity. By increasing affinity of binding output will go up, and conversely by decreasing affinity of binding output will go down.

In some embodiments, there is a spacer between binding sites. In some embodiments, a promoter comprises at least one spacer between two regulatory elements. In some embodiments, a spacer comprises at least 1, 2, 3, 4, 5, 6, 7, 8, 9, or 10 nucleotides. Each possibility represents a separate embodiment of the invention. In some embodiments, the spacer is at least 3 nucleotides. In some embodiments, the size or nucleotide makeup of a spacer enhances or inhibits binding to a regulatory element. In some embodiments, the size or nucleotide makeup of a spacer enhances or inhibits the regulatory effect of a regulatory element.

In some embodiments, the transcriptional regulatory element is a promoter. In some embodiments, the transcriptional regulatory element comprises at least one binding site for the DNA binding domain. In some embodiments, at least one binding site is 1, 2, 3, 4, 5, 6, 7, 8, 9 or 10 binding sites. Each possibility represents a separate embodiment of the invention. In some embodiments, at least one binding site is 3 binding sites. In some embodiments, at least one binding site is 8 binding sites. In some embodiments, the binding sites are separated by spacers. In some embodiments, a spacer comprises or consists of 2 nucleotides. In some embodiments, a spacer comprises or consists of 7 nucleotides. In some embodiments, a spacer comprises or consists of 11 nucleotides. In some embodiments, a spacer comprises or consists of 6 nucleotides. In some embodiments, the spacers are all the same size. In some embodiments, the spacers are different sizes. In some embodiments, the transcription regulatory element comprises binding sites for the DNA binding domain and a promoter. In some embodiments, the binding sites for the DNA binding domain are 5′ to the promoter. In some embodiments, the most 3′ binding site is separated from the promoter by a spacer.

In some embodiments, the transcription regulatory element comprises 8 Gal4-binding sites. In some embodiments, the transcription regulatory element comprises the nucleotide sequence cggagtactgtcctccgagcggagtactgtcctccgactcgagcggagtactgtcctccgatcggagtactgtcctccgcgaattc cggagtactgtcctccgaagacgctagacggagtactgtcctccgagcggagtactgtcctccgactcgagcggagtactgtcctc cg (SEQ ID NO: 45). In some embodiments, the transcription regulatory element comprises 3 Gal4-binding sites. In some embodiments, the transcription regulatory element comprises the nucleotide sequence cggagtactgtcctccgagcggagtactgtcctccgactcgagcggagtactgtcctccg (SEQ ID NO: 46).

In some embodiments, the polynucleotide is the output. In some embodiments, the third nucleic acid molecule is an output construct. In some embodiments, the polynucleotide is a therapeutic molecule. In some embodiments, the polynucleotide encodes a protein. In some embodiments, the polynucleotide comprises an open reading frame encoding a protein. In some embodiments, the protein is a therapeutic protein. In some embodiments, the molecule is a protein. In some embodiments, the molecule is an RNA. In some embodiments, the polynucleotide comprises a third open reading frame. In some embodiments, the polynucleotide encodes a protein. In some embodiments, the polynucleotide comprises a sequence of an RNA. In some embodiments, the RNA is a non-coding RNA. In some embodiments, the non-coding RNA is a regulatory RNA. In some embodiments, the polynucleotide comprises a sequence of a regulatory RNA. In some embodiments, the polynucleotide encodes a regulatory RNA. In some embodiments, the polynucleotide is transcribable to an RNA. In some embodiments, the RNA is a regulatory RNA. Examples of regulatory RNAs include but are not limited to small interfering RNAs (siRNAs), microRNAs (miRs), short hairpin RNAs (shRNAs), antisense oligonucleotides (ASOs), ribozymes and long non-coding RNAs (lncRNAs). In some embodiments, the RNA is a therapeutic RNA.

In some embodiments, the protein is a therapeutic protein. In some embodiments, the output is a therapeutic output. In some embodiments, the therapeutic output is gene therapy. In some embodiments, the output is a drug. In some embodiments, the protein is a naturally occurring protein. In some embodiments, the protein is a synthetic protein. In some embodiments, the therapeutic molecule is an immunotherapy. In some embodiments, an immunotherapy is an immunomodulator. In some embodiments, an immunotherapy is an immunostimulant. In some embodiments, the immunotherapy is a cytokine. In some embodiments, the therapeutic molecule is toxic. In some embodiments, the therapeutic molecule induces apoptosis. In some embodiments, the therapeutic protein is a pro-apoptotic protein. In some embodiments, the therapeutic protein is an anticancer protein. In some embodiments, the therapeutic protein is a toxin. In some embodiments, the therapeutic protein induces cell cycle arrest. In some embodiments, the therapeutic molecule is an antibody or antigen binding domain. In some embodiments, the therapeutic molecule is a protein domain. In some embodiments, the therapeutic molecule is a full-length protein. In some embodiments, the therapeutic molecule is a binding domain. In some embodiments, the therapeutic molecule is a fusion protein. In some embodiments, the therapeutic protein is a receptor. In some embodiments, the receptor is a chimeric antigen receptor. In some embodiments, the therapeutic protein is an immunogenic cell death inducer.

In some embodiments, the therapeutic protein is an immunomodulatory protein. In some embodiments, the immunomodulatory protein is a cytokine. In some embodiments, the cytokine is a proinflammatory cytokine. Examples of proinflammatory cytokines include, but are not limited to interleukin-1 (IL-1), IL-2, IL-6, IL-12, IL-15, IL-17, IL-18, interferon gamma and tumor necrosis factor alpha. In some embodiments, the therapeutic protein is IL-2. In some embodiments, the therapeutic protein is IL-15. In some embodiments, the cytokine is a chemokine. In some embodiments, the chemokine is CCL21. In some embodiments, the cytokine is a stimulatory cytokine. Examples of stimulatory cytokines include, but are not limited to GM-CSF, G-CSF, FLT3LG, and CSF-1.

In some embodiments, IL-2 is encoded by a nucleic acid sequence comprising gcacctacttcaagttctacaaagaaaacacagctacaactggagcatttactgctggatttacagatgattttgaatggaattaataat tacaagaatcccaaactcaccaggatgctcacatttaagttttacatgcccaagaaggccacagaactgaaacatcttcagtgtctag aagaagaactcaaacctctggaggaagtgctaaatttagctcaaagcaaaaactttcacttaagacccagggacttaatcagcaata tcaacgtaatagttctggaactaaagggatctgaaacaacattcatgtgtgaatatgctgatgagacagcaaccattgtagaatttctg aacagatggattaccttttgtcaaagcatcatctcaacactgact (SEQ ID NO: 49). In some embodiments, IL-2 is encoded by a nucleic acid sequence consisting of SEQ ID NO: 49. In some embodiments, IL-2 is encoded by a sequence with at least 85% identity to SEQ ID NO: 49 and which encodes a protein with proinflammatory cytokine function. In some embodiments, IL-2 comprises the amino acid sequence APTSSSTKKTQLQLEHLLLDLQMILNGINNYKNPKLTRMLTFKFYMPKKATELKH LQCLEEELKPLEEVLNLAQSKNFHLRPRDLISNINVIVLELKGSETTFMCEYADETA TIVEFLNRWITFCQSIISTLT (SEQ ID NO: 50). In some embodiments, IL-2 consists of SEQ ID NO: 50. In some embodiments, IL-2 comprises a sequence with at least 85% identity to SEQ ID NO: 50 and which retains proinflammatory cytokine function. In some embodiments, SEQ ID NO: 49 further comprises a 5′ atg codon. In some embodiments, SEQ ID NO: 50 further comprises an N-terminal methionine residue.

In some embodiments, IL-15 is encoded by a nucleic acid sequence comprising ggcattcatgtcttcattttgggctgtttcagtgcagggcttcctaaaacagaagccaactgggtgaatgtaataagtgatttgaaaaa aattgaagatcttattcaatctatgcatattgatgctactttatatacggaaagtgatgttcaccccagttgcaaagtaacagcaatgaa gtgctttctcttggagttacaagttatttcacttgagtccggagatgcaagtattcatgatacagtagaaaatctgatcatcctagcaaac aacagtttgtcttctaatgggaatgtaacagaatctggatgcaaagaatgtgaggaactggaggaaaaaaatattaaagaatttttgc agagttttgtgcatattgtccaaatgttcatcaacacttct (SEQ ID NO: 47). In some embodiments, IL-15 is encoded by a nucleic acid sequence consisting of SEQ ID NO: 47. In some embodiments, IL-15 is encoded by a sequence with at least 85% identity to SEQ ID NO: 47 and which encodes a protein with proinflammatory cytokine function. In some embodiments, IL-15 comprises the amino acid sequence GIHVFILGCFSAGLPKTEANWVNVISDLKKIEDLIQSMHIDATLYTESDVHPSCKVT AMKCFLLELQVISLESGDASIHDTVENLIILANNSLSSNGNVTESGCKECEELEEKNI KEFLQSFVHIVQMFINTS (SEQ ID NO: 48). In some embodiments, IL-15 consists of SEQ ID NO: 48. In some embodiments, IL-15 comprises a sequence with at least 85% identity to SEQ ID NO: 48 and which retains proinflammatory cytokine function. In some embodiments, SEQ ID NO: 47 further comprises a 5′ ATG codon. In some embodiments, SEQ ID NO: 48 further comprises an N-terminal methionine residue.

In some embodiments, IL-15 is encoded by a nucleic acid sequence comprising gccaactgggtgaatgtaataagtgatttgaaaaaaattgaagatcttattcaatctatgcatattgatgctactttatatacggaaagtg atgttcaccccagttgcaaagtaacagcaatgaagtgctttctcttggagttacaagttatttcacttgagtccggagatgcaagtattc atgatacagtagaaaatctgatcatcctagcaaacaacagtttgtcttctaatgggaatgtaacagaatctggatgcaaagaatgtga ggaactggaggaaaaaaatattaaagaatttttgcagagttttgtacatattgtccaaatgttcatcaacacttct (SEQ ID NO: 70). In some embodiments, IL-15 is encoded by a nucleic acid sequence consisting of SEQ ID NO: 70. In some embodiments, IL-15 is encoded by a sequence with at least 85% identity to SEQ ID NO: 70 and which encodes a protein with proinflammatory cytokine function. In some embodiments, IL-15 comprises the amino acid sequence ANWVNVISDLKKIEDLIQSMHIDATLYTESDVHPSCKVTAMKCFLLELQVISLESG DASIHDTVENLIILANNSLSSNGNVTESGCKECEELEEKNIKEFLQSFVHIVQMFINT S (SEQ ID NO: 71). In some embodiments, IL-15 consists of SEQ ID NO: 71. In some embodiments, IL-15 comprises a sequence with at least 85% identity to SEQ ID NO: 71 and which retains proinflammatory cytokine function. In some embodiments, SEQ ID NO: 70 further comprises a 5′ ATG codon. In some embodiments, SEQ ID NO: 71 further comprises an N-terminal methionine residue.

In some embodiments, the therapeutic protein is Granulocyte-macrophage colony-stimulating factor (GM-CSF). In some embodiments, GM-CSF is encoded by a sequence comprising gcacctgccagatctcctagtcccagcacacaaccctgggagcatgtgaatgccatccaggaggccaggagactcctgaacctg agtagagatactgctgctgagatgaatgaaacagtggaagttatctctgaaatgtttgacctccaagagcctacttgccttcagacca gactggagctgtataagcagggactgcggggcagcctcaccaagctcaagggccccttgaccatgatggctagccactacaaac agcactgccctccaacaccagaaacttcctgtgcaacccagattatcacctttgaaagtttcaaagagaacctgaaggacttcctgct tgtcattccctttgactgctgggagccagtgcaggag (SEQ ID NO: 74). In some embodiments, GM-CSF is encoded by a sequence consisting of SEQ ID NO: 74. In some embodiments, GM-CSF is encoded by a sequence with at least 85% identity to SEQ ID NO: 74 and which encodes a protein with the ability to bind GM-CSF receptor (GM-CSFR). In some embodiments, GM-CSF is encoded by a sequence with at least 85% identity to SEQ ID NO: 74 and which encodes a protein with the ability to bind GM-CSFR and induce signaling by the engaged receptor. In some embodiments, GM-CSF comprises APARSPSPSTQPWEHVNAIQEARRLLNLSRDTAAEMNETVEVISEMFDLQEPTCLQ TRLELYKQGLRGSLTKLKGPLTMMASHYKQHCPPTPETSCATQIITFESFKENLKD FLLVIPFDCWEPVQE (SEQ ID NO: 75). In some embodiments, GM-CSF consists of SEQ ID NO: 75. In some embodiments, GM-CSF comprises an amino acid sequence with at least 85% identity to SEQ ID NO: 75 and which retains the ability to bind GM-CSFR. In some embodiments, GM-CSF comprises an amino acid sequence with at least 85% identity to SEQ ID NO: 75 and which retains the ability to bind GM-CSFR and induce signaling by the bound receptor. In some embodiments, SEQ ID NO: 74 further comprises a 5′ ATG codon. In some embodiments, SEQ ID NO: 75 further comprises an N-terminal methionine residue.

In some embodiments, the therapeutic protein is Fms-related tyrosine kinase 3 ligand (FLT3LG). In some embodiments, FLT3LG is encoded by a sequence comprising acccaggactgctccttccaacacagccccatctcctccgacttcgctgtcaaaatccgtgagctgtctgactacctgcttcaagatt acccagtcaccgtggcctccaacctgcaggacgaggagctctgcgggggcctctggcggctggtcctggcacagcgctggatg gagcggctcaagactgtcgctgggtccaagatgcaaggcttgctggagcgcgtgaacacggagatacactttgtcaccaaatgtg cctttcagcccccccccagctgtcttcgcttcgtccagaccaacatctcccgcctcctgcaggagacctccgagcagctggtggcg ctgaagccctggatcactcgccagaacttctcccggtgcctggagctgcagtgtcagcccgactcctcaaccctgccacccccatg gagtccccggcccctggaggccacagccccgacagccccgcagccccctctgctcctcctactgctgctgcccgtgggcctcct gctgctggccgctgcctggtgcctgcactggcagaggacgcggcggaggacaccccgccctggggagcaggtgccccccgtc cccagtccccaggacctgctgcttgtggagcac (SEQ ID NO: 78). In some embodiments, FLT3LG is encoded by a sequence consisting of SEQ ID NO: 78. In some embodiments, FLT3LG is encoded by a sequence with at least 85% identity to SEQ ID NO: 78 and which encodes a protein with the ability to bind CD40. In some embodiments, FLT3LG is encoded by a sequence with at least 85% identity to SEQ ID NO: 78 and which encodes a protein with the ability to bind fms like tyrosine kinase 3 (FLT3) and induce signaling by the engaged receptor. In some embodiments, FLT3LG comprises TQDCSFQHSPISSDFAVKIRELSDYLLQDYPVTVASNLQDEELCGGLWRLVLAQR WMERLKTVAGSKMQGLLERVNTEIHFVTKCAFQPPPSCLRFVQTNISRLLQETSEQ LVALKPWITRQNFSRCLELQCQPDSSTLPPPWSPRPLEATAPTAPQPPLLLLLLLPV GLLLLAAAWCLHWQRTRRRTPRPGEQVPPVPSPQDLLLVEH (SEQ ID NO: 79). In some embodiments, FLT3LG consists of SEQ ID NO: 79. In some embodiments, FLT3LG comprises an amino acid sequence with at least 85% identity to SEQ ID NO: 79 and which retains the ability to bind FLT3. In some embodiments, FLT3LG comprises an amino acid sequence with at least 85% identity to SEQ ID NO: 79 and which retains the ability to bind FLT3 and induce signaling by the bound receptor. In some embodiments, SEQ ID NO: 78 further comprises a 5′ ATG codon. In some embodiments, SEQ ID NO: 79 further comprises an N-terminal methionine residue.

In some embodiments, the therapeutic protein is C—C motif chemokine 21 (CCL21). In some embodiments, CCL21 is encoded by a sequence comprising agcgatggtggcgcacaggactgttgcctgaagtattcacagcggaaaatcccagctaaagtggttcggagctatcgcaagcagg aaccttccttggggtgttcaatcccagctatcctctttcttccaagaaagcgctctcaggccgagctgtgtgctgacccaaaagagct gtgggtccagcagttgatgcagcacttggacaagacgccgtctccccagaaacccgcccagggctgcagaaaagataggggcg cctcaaaaacaggcaaaaaagggaaaggtagcaaaggttgcaaacgcaccgaacgaagccagacgccaaaagggcca (SEQ ID NO: 86). In some embodiments, CCL21 is encoded by a sequence consisting of SEQ ID NO: 86. In some embodiments, CCL21 is encoded by a sequence with at least 85% identity to SEQ ID NO: 86 and which encodes a protein with the ability to bind C—C chemokine receptor type 7 (CCR7). In some embodiments, CCL21 is encoded by a sequence with at least 85% identity to SEQ ID NO: 86 and which encodes a protein with the ability to bind CCR7 and induce signaling by the engaged receptor. In some embodiments, CCL21 comprises SDGGAQDCCLKYSQRKIPAKVVRSYRKQEPSLGCSIPAILFLPRKRSQAELCADPKE LWVQQLMQHLDKTPSPQKPAQGCRKDRGASKTGKKGKGSKGCKRTERSQTPKG P (SEQ ID NO: 87). In some embodiments, CCL21 consists of SEQ ID NO: 87. In some embodiments, CCL21 comprises an amino acid sequence with at least 85% identity to SEQ ID NO: 87 and which retains the ability to bind CCR7. In some embodiments, CCL21 comprises an amino acid sequence with at least 85% identity to SEQ ID NO: 87 and which retains the ability to bind CCR7 and induce signaling by the bound receptor. In some embodiments, SEQ ID NO: 86 further comprises a 5′ ATG codon. In some embodiments, SEQ ID NO: 87 further comprises an N-terminal methionine residue.

In some embodiments, the therapeutic protein is a therapeutic single chain variable region. In some embodiments, the therapeutic protein is a single domain antibody. In some embodiments, the therapeutic protein is a VHH. In some embodiments, the therapeutic protein is a CD3 binding agent. In some embodiments, the therapeutic protein is an anti-CD3 scFv. In some embodiments, the binding agent is selected from an scFv, a single domain antibody and a VHH. In some embodiments, CD3 is CD3 epsilon (CD3e). CD3e is well known in the art. The human gene can be found at Entrez gene #916 and the human protein can be found at Uniprot #P07766. The mRNA sequence for human CD3e can be found in RefSeq ID NM_000733 and the human protein can be found in RefSeq ID NP_000724. In some embodiments, the anti-CD3e scFv is encoded by a sequence comprising caagtgcagcttcagcagagcggcgctgagctggcccggcccggggcctctgtgaaaatgagctgcaaggcttccggttacacc tttacaaggtacacaatgcactgggtgaagcagcggccagggcaggggctcgagtggatcggctacataaacccatctcgggga tatacaaactacaatcaaaagttcaaagacaaggccacactgacaacggacaagtcaagcagcacagcttacatgcaactgagct ctctgacatccgaggatagcgccgtttactattgcgccagatactatgatgaccactattgcttggactactggggacagggaacaa ccctcacagttagctccggcggggggggcagtggaggtggaggatctgggggcggcggtagtcagatcgtcctcacacagtct ccggccataatgtccgcctcccccggagagaaggttactatgacatgttccgcatcttcctctgtgtcatatatgaattggtaccagca gaagagtggcacctctcctaaacgctggatttacgatacctctaaactggcgtccggggtgcctgcacatttccgcggatccggctc cggtacgagttattcactcacaatatctggaatggaggccgaagatgccgctacttactactgccaacaatggtcaagcaacccctt cactttcgggagcgggacaaagctggagatcaac (SEQ ID NO: 51). In some embodiments, the anti-CD3e scFv is encoded by a sequence consisting of SEQ ID NO: 51. In some embodiments, the anti-CD3e scFv is encoded by a sequence comprising at least 85% identity to SEQ ID NO: 51 and encoding a protein that retains the 3 heavy chain CDRs and 3 light chain CDRs such that it retains binding to CD3e. In some embodiments, the anti-CD3e scFv is encoded by a sequence comprising at least 85% identity to SEQ ID NO: 51 and encoding a protein that retains the 3 heavy chain CDRs and 3 light chain CDRs such that it retains T cell stimulatory activity. In some embodiments, the scFv comprises the amino acid sequence QVQLQQSGAELARPGASVKMSCKASGYTFTRYTMHWVKQRPGQGLEWIGYINPS RGYTNYNQKFKDKATLTTDKSSSTAYMQLSSLTSEDSAVYYCARYYDDHYCLDY WGQGTTLTVSSGGGGSGGGGSGGGGSQIVLTQSPAIMSASPGEKVTMTCSASSSV SYMNWYQQKSGTSPKRWIYDTSKLASGVPAHFRGSGSGTSYSLTISGMEAEDAAT YYCQQWSSNPFTFGSGTKLEIN (SEQ ID NO: 52). In some embodiments, the anti-CD3e scFv consists of SEQ ID NO: 52. In some embodiments, the scFv comprises a sequence with at least 85% identity to SEQ ID NO: 52 and which retains the 3 heavy chain CDRs and 3 light chain CDRs and retains binding to CD3e. In some embodiments, the anti-CD3e scFv is a stimulatory scFv. In some embodiments, a stimulatory scFv stimulates T cell activation. In some embodiments, the activation is stimulated upon binding of the CD3e on T cells by the scFv. In some embodiments, the scFv comprises a sequence with at least 85% identity to SEQ ID NO: 52 and which retains the 3 heavy chain CDRs and 3 light chain CDRs and retains T cell activation ability.

In some embodiments, the therapeutic protein is an immune checkpoint inhibitor. In some embodiments, the therapeutic protein is an immune checkpoint blockade molecule. In some embodiments, the therapeutic protein is a single chain antibody immune checkpoint inhibitor. Examples of immune checkpoint proteins that can be blocked by the inhibitor include, but are not limited to PD-1, PD-L1, CTLA-4, LAG-3, TIM-3, BTLA, HVEM, ICOS, and TIGIT. In some embodiments, the therapeutic protein is a Programmed cell death protein 1 (PD-1) binding agent. In some embodiments, the therapeutic protein is an anti-PD-1 scFv. In some embodiments, the binding agent is selected from an scFv, a single domain antibody and a VHH. PD-1 is well known in the art. The human gene can be found at Entrez gene #5133, and the human protein can be found at Uniprot #Q15116. The mRNA sequence for human PD-1 can be found in RefSeq ID NM_005018 and the human protein can be found in RefSeq ID NP_005009. In some embodiments, the anti-PD-1 scFv is based off the variable regions of the anti-PD-1 antibody pembrolizumab. In some embodiments, the scFV comprises a heavy chain variable region of pembrolizumab. In some embodiments, the heavy chain variable region of pembrolizumab comprises or consists of SEQ ID NO: 68. In some embodiments, the scFV comprises a light chain variable region of pembrolizumab. In some embodiments, the light chain variable region of pembrolizumab comprises or consists of SEQ ID NO: 69. In some embodiments, the anti-PD-1 scFv is encoded by a sequence comprising atgggagttaaggtattgttcgcattgatttgcatagccgtggccgaagctcaggtgcagctcgtagagagcgggggaggtgtcgt tcagccagggaggagtctgcggctggactgcaaagcctccgggattactttctcaaacagcgggatgcactgggtgaggcaggc tcccggaaagggcctcgagtgggtggcggtaatatggtacgacgggagcaaacgctactacgcagacagtgtgaaaggaaggt tcactatttctcgcgacaattcaaagaacaccctcttcctgcagatgaacagtctgcgggcagaagacaccgctgtctattactgtgc caccaatgacgattactggggccagggtaccctcgtgaccgtgtctagcgcctctaccaaaggaccatcagtttttcctcttgctccc tgcagtcgctcaaccagcgagagcacagcagcgctgggatgtctggtgaaggattattttcctgagcccgtgactgtgagctggaa ttcaggcgccctgacgtccggcgtccatacattcccagcggtacttcaaagtagcgggttgtactctctctctagcgtggtaaccgta ccgagctcctccctggggacgaaaacgtatacatgtaatgtcgatcacaaaccatctaacacaaaagtggacaaacgcgttgagtc caagtatggccctccatgcccaccctgccccgcaccggagtttctgggcgggcccagtgtctttctgttcccaccgaagcctaagg acacgttgatgatctcaagaacacctgaagtcacctgcgtagtcgtggacgtttctcaggaggatcccgaggtccaattcaattggt acgttgatggagtggaggtccacaacgcaaagacaaagccgcgcgaagaacagttcaattctacttaccgcgttgttagcgtgctg actgtgctccaccaagactggctgaatggtaaggagtataagtgcaaggtgagcaataagggattgccatctagcatcgaaaaga caatatccaaagccaagggccaaccacgagagccacaagtgtacacgttgcctccctcacaagaagagatgaccaagaatcaag tgagcctcacttgcctggtcaagggattctacccttctgatatcgcagtggagtgggagtccaatggacagcccgagaacaactac aagacaacacccccagtgctggattccgacggctcattcttcttgtatagccggctgacagtggacaagagcaggtggcaggaag gaaatgtcttctcctgctccgtgatgcacgaggccctccacaaccactacactcagaaatctctctctctttcactcggtaaaggaag cggagctactaacttcagcctgctgaagcaggctggagacgtggaggagaaccctggacctggagttaaggtattgttcgcattga tttgcatagccgtggccgaagctgaaatagtgctgacccagtcccccgccaccctctctctctcacccggagagagggcaactctg tcctgcagggcctcccaatccgtaagcagctatctggcttggtaccagcaaaaacccggacaggcaccccggctgttgatctatga cgcctccaaccgagccactggcatccccgccagattctccggatctggatcagggactgattttaccctgactatcagttctctgga acctgaagattttgcagtttattattgtcagcaaagctctaactggccaagaacctttggccagggcacgaaggtcgagataaaaact gtcgcggccccaagcgtctttatctttccgccgtctgatgaacagctgaaatcagggaccgcctctgtggtgtgtctgctgaacaact tctacccaagggaagctaaggtgcaatggaaagtagacaatgccttgcagagcggcaactctcaggagagcgtaaccgaacag gatagtaaggattcaacttacagtctgagctctaccctgactctgagtaaggccgattacgagaagcataaagtctacgcatgcgag gtcacacatcaaggtctttcatctcccgtgacaaagtcctttaatcgaggggaatgc (SEQ ID NO: 82). In some embodiments, the anti-PD-1 scFv is encoded by a sequence consisting of SEQ ID NO: 82. In some embodiments, the anti-PD-1 scFv is encoded by a sequence comprising at least 85% identity to SEQ ID NO: 82 and encoding a protein that retains the 3 heavy chain CDRs and 3 light chain CDRs such that it retains binding to Programmed death-ligand 1 (PD-L1). In some embodiments, the anti-PD-1 scFv is encoded by a sequence comprising at least 85% identity to SEQ ID NO: 82 and encoding a protein that retains the 3 heavy chain CDRs and 3 light chain CDRs such that it retains immune checkpoint blockade function. In some embodiments, the scFv comprises the amino acid sequence MGVKVLFALICIAVAEAQVQLVESGGGVVQPGRSLRLDCKASGITFSNSGMHWVR QAPGKGLEWVAVIWYDGSKRYYADSVKGRFTISRDNSKNTLFLQMNSLRAEDTA VYYCATNDDYWGQGTLVTVSSASTKGPSVFPLAPCSRSTSESTAALGCLVKDYFP EPVTVSWNSGALTSGVHTFPAVLQSSGLYSLSSVVTVPSSSLGTKTYTCNVDHKPS NTKVDKRVESKYGPPCPPCPAPEFLGGPSVFLFPPKPKDTLMISRTPEVTCVVVDVS QEDPEVQFNWYVDGVEVHNAKTKPREEQFNSTYRVVSVLTVLHQDWLNGKEYK CKVSNKGLPSSIEKTISKAKGQPREPQVYTLPPSQEEMTKNQVSLTCLVKGFYPSDI AVEWESNGQPENNYKTTPPVLDSDGSFFLYSRLTVDKSRWQEGNVFSCSVMHEA LHNHYTQKSLSLSLGKGSGATNFSLLKQAGDVEENPGPGVKVLFALICIAVAEAEI VLTQSPATLSLSPGERATLSCRASQSVSSYLAWYQQKPGQAPRLLIYDASNRATGI PARFSGSGSGTDFTLTISSLEPEDFAVYYCQQSSNWPRTFGQGTKVEIKTVAAPSVF IFPPSDEQLKSGTASVVCLLNNFYPREAKVQWKVDNALQSGNSQESVTEQDSKDS TYSLSSTLTLSKADYEKHKVYACEVTHQGLSSPVTKSFNRGEC (SEQ ID NO: 83). In some embodiments, the anti-PD-1 scFv consists of SEQ ID NO: 83. In some embodiments, the scFv comprises a sequence with at least 85% identity to SEQ ID NO: 83 and which retains the 3 heavy chain CDRs and 3 light chain CDRs and retains binding to PD-L1. In some embodiments, the anti-PD-1 scFv is an immune checkpoint inhibitor. In some embodiments, an immune checkpoint inhibitor is suitable for immune checkpoint blockade. In some embodiments, the scFv comprises a sequence with at least 85% identity to SEQ ID NO: 83 and which retains the 3 heavy chain CDRs and 3 light chain CDRs and retains immune checkpoint inhibition.

In some embodiments, the therapeutic protein is a secreted protein. In some embodiments, the therapeutic protein is a cell surface protein. In some embodiments, the therapeutic protein is configured for display on the cell surface. In some embodiments, the scFv is configured for display on the cell surface. In some embodiments, the therapeutic protein is a fusion protein of an scFv and a membrane anchor. In some embodiments, the therapeutic protein comprises a membrane anchor domain. In some embodiments, the membrane anchor is a Duffy antigen/chemokine receptor (DARC). In some embodiments, the scFv is N-terminal to the membrane anchor. In some embodiments, the scFv is linked to the membrane anchor by a linker. In some embodiments, the DARC is encoded by a sequence comprising gcaggtatggcctcctctgggtatgtcctccaggcggagctctccccctcaactgagaactcaagtcagctggacttcgaagatgta tggaattcttcctatggtgtgaatgattccttcccagatggagactatgatgccaacctggaagcagctgccccctgccactcctgta acctgctggatgactctgcactgcccttcttcatcctcaccagtgtcctgggtatcctagctagcagcactgtcctcttcatgcttttcag acctctcttccgctggcagctctgccctggctggcctgtcctggcacagctggctgtgggcagtgccctcttcagcattgtggtgcc cgtcttggccccagggctaggtagcactcgcagctctgccctgtgtagcctgggctactgtgtctggtatggctcagcctttgccca ggctttgctgctagggtgccatgcctccctgggccacagactgggtgcaggccaggtcccaggcctcaccctggggctcactgtg ggaatttggggagtggctgccctactgacactgcctgtcaccctggccagtggtgcttctggtggactctgcaccctgatatacagc acggagctgaaggctttgcaggccacacacactgtagcctgtcttgccatctttgtcttgttgccattgggtttgtttggagccaaggg gctgaagaaggcattgggtatggggccaggcccctggatgaatatcctgtgggcctggtttattttctggtggcctcatggggtggtt ctaggactggatttcctggtgaggtccaagctgttgctgttgtcaacatgtctggcccagcaggctctggacctgctgctgaacctgg cagaagccctggcaattttgcactgtgtggctacgcccctgctcctcgccctattctgccaccaggccacccgcaccctcttgccct ctctgcccctccctgaaggatggtcttctcatctggacacccttggaagcaaatcc (SEQ ID NO: 53). In some embodiments, the DARC is encoded by a sequence consisting of SEQ ID NO: 53. In some embodiments, the DARC is encoded by a sequence with at least 85% identity to SEQ ID NO: 53 and that encodes a protein with membrane anchoring function. In some embodiments, the DARC comprises the amino acid sequence AGMASSGYVLQAELSPSTENSSQLDFEDVWNSSYGVNDSFPDGDYDANLEAAAP CHSCNLLDDSALPFFILTSVLGILASSTVLFMLFRPLFRWQLCPGWPVLAQLAVGS ALFSIVVPVLAPGLGSTRSSALCSLGYCVWYGSAFAQALLLGCHASLGHRLGAGQ VPGLTLGLTVGIWGVAALLTLPVTLASGASGGLCTLIYSTELKALQATHTVACLAI FVLLPLGLFGAKGLKKALGMGPGPWMNILWAWFIFWWPHGVVLGLDFLVRSKLL LLSTCLAQQALDLLLNLAEALAILHCVATPLLLALFCHQATRTLLPSLPLPEGWSSH LDTLGSKS (SEQ ID NO: 54). In some embodiments, the DARC consists of SEQ ID NO: 54. In some embodiments, the DARC comprises at least 85% identity to SEQ ID NO: 54 and retains membrane anchorage function.

In some embodiments, the therapeutic protein is an immune cell stimulating molecule. In some embodiments, the immune cell is a cytotoxic immune cell. In some embodiments, the immune cell is selected from a T cell, a natural killer cell (NK) cell, a B cell, a macrophage, a dendritic cell and an antigen presenting cell. In some embodiments, a cytotoxic immune cell is selected from a T cell and an NK cell. In some embodiments, the T cell is a CD8 positive T cell. In some embodiments, the therapeutic protein is an immune cell activating molecule. In some embodiments, stimulating is activating. In some embodiments, the immune cell stimulating protein is a cytokine. In some embodiments, the immune cell activating protein is selected from: CD40L, MICA, ULBP1, 4-1BBL and OX40L.

In some embodiments, the therapeutic protein is CD40 ligand (CD40L). In some embodiments, CD40L is encoded by a sequence comprising atgatcgaaacatacaaccaaacttctccccgatctgcggccactggactgcccatcagcatgaaaatttttatgtatttacttactgttt ttcttatcacccagatgattgggtcagcactttttgctgtgtatcttcatagaaggttggacaagatagaagatgaaaggaatcttcatg aagattttgtattcatgaaaacgatacagagatgcaacacaggagaaagatccttatccttactgaactgtgaggagattaaaagcca gtttgaaggctttgtgaaggatataatgttaaacaaagaggagacgaagaaagaaaacagctttgaaatgcaaaaaggtgatcaga atcctcaaattgcggcacatgtcataagtgaggccagcagtaaaacaacatctgtgttacagtgggctgaaaaaggatactacacc atgagcaacaacttggtaaccctggaaaatgggaaacagctgaccgttaaaagacaaggactctattatatctatgcccaagtcacc ttctgttccaatcgggaagcttcgagtcaagctccatttatagccagcctctgcctaaagtcccccggtagattcgagagaatcttact cagagctgcaaatacccacagttccgccaaaccttgcgggcaacaatccattcacttgggaggagtatttgaattgcaaccaggtg cttcggtgtttgtcaatgtgactgatccaagccaagtgagccatggcactggcttcacgtcctttggcttactcaaactc (SEQ ID NO: 66). In some embodiments, CD40L is encoded by a sequence consisting of SEQ ID NO: 66. In some embodiments, CD40L is encoded by a sequence with at least 85% identity to SEQ ID NO: 66 and which encodes a protein with the ability to bind CD40. In some embodiments, CD40L is encoded by a sequence with at least 85% identity to SEQ ID NO: 66 and which encodes a protein with the ability to bind CD40 and induce signaling by the bound CD40. In some embodiments, CD40L comprises MIETYNQTSPRSAATGLPISMKIFMYLLTVFLITQMIGSALFAVYLHRRLDKIEDER NLHEDFVFMKTIQRCNTGERSLSLLNCEEIKSQFEGFVKDIMLNKEETKKENSFEM QKGDQNPQIAAHVISEASSKTTSVLQWAEKGYYTMSNNLVTLENGKQLTVKRQG LYYIYAQVTFCSNREASSQAPFIASLCLKSPGRFERILLRAANTHSSAKPCGQQSIHL GGVFELQPGASVFVNVTDPSQVSHGTGFTSFGLLKL (SEQ ID NO: 67). In some embodiments, CD40L consists of SEQ ID NO: 67. In some embodiments, CD40L comprises an amino acid sequence with at least 85% identity to SEQ ID NO: 67 and which retains the ability to bind CD40. In some embodiments, CD40L comprises an amino acid sequence with at least 85% identity to SEQ ID NO: 67 and which retains the ability to bind CD40 and induce signaling by the bound CD40. In some embodiments, SEQ ID NO: 66 further comprises a 5′ ATG codon. In some embodiments, SEQ ID NO: 67 further comprises an N-terminal methionine residue.

In some embodiments, the therapeutic protein is MHC class I polypeptide-related sequence A (MICA). In some embodiments, MICA is encoded by a sequence comprising ccccacagtcttcgttataacctcacggtgctgtcctgggatggatctgtgcagtcagggtttctcactgaggtacatctggatggtca gcccttcctgcgctgtgacaggcagaaatgcagggcaaagccccagggacagtgggcagaagatgtcctgggaaataagacat gggacagagagaccagagacttgacagggaacggaaaggacctcaggatgaccctggctcatatcaaggaccagaaagaagg cttgcattccctccaggagattagggtctgtgagatccatgaagacaacagcaccaggagctcccagcatttctactacgatgggg agctcttcctctcccaaaacctggagactaaggaatggacaatgccccagtcctccagagctcagaccttggccatgaacgtcagg aatttcttgaaggaagatgccatgaagaccaagacacactatcacgctatgcatgcagactgcctgcaggaactacggcgatatct aaaatccggcgtagtcctgaggagaacagtgccccccatggtgaatgtcacccgcagcgaggcctcagagggcaacattaccgt gacatgcagggcttctggcttctatccctggaatatcacactgagctggcgtcaggatggggtatctttgagccacgacacccagc agtggggggatgtcctgcctgatgggaatggaacctaccagacctgggtggccaccaggatttgccaaggagaggagcagagg ttcacctgctacatggaacacagcgggaatcacagcactcaccctgtgccctctgggaaagtgctggtgcttcagagtcattggca gacattccatgtttctgctgttgctgctgctgctatttttgttattattattttctatgtccgttgttgtaagaagaaaacatcagctgcagag ggtccagagctcgtgagcctgcaggtcctggatcaacacccagttgggacgagtgaccacagggatgccacacagctcggattt cagcctctgatgtcagatcttgggtccactggctccactgagggcgcc (SEQ ID NO: 90). In some embodiments, MICA is encoded by a sequence consisting of SEQ ID NO: 90. In some embodiments, MICA is encoded by a sequence with at least 85% identity to SEQ ID NO: 90 and which encodes a protein with the ability to bind natural-killer group 2, member D (NKG2D). In some embodiments, MICA is encoded by a sequence with at least 85% identity to SEQ ID NO: 90 and which encodes a protein with the ability to bind NKG2D and induce signaling by the engaged receptor. In some embodiments, MICA comprises PHSLRYNLTVLSWDGSVQSGFLTEVHLDGQPFLRCDRQKCRAKPQGQWAEDVLG NKTWDRETRDLTGNGKDLRMTLAHIKDQKEGLHSLQEIRVCEIHEDNSTRSSQHF YYDGELFLSQNLETKEWTMPQSSRAQTLAMNVRNFLKEDAMKTKTHYHAMHAD CLQELRRYLKSGVVLRRTVPPMVNVTRSEASEGNITVTCRASGFYPWNITLSWRQ DGVSLSHDTQQWGDVLPDGNGTYQTWVATRICQGEEQRFTCYMEHSGNHSTHPV PSGKVLVLQSHWQTFHVSAVAAAAIFVIIIFYVRCCKKKTSAAEGPELVSLQVLDQ HPVGTSDHRDATQLGFQPLMSDLGSTGSTEGA (SEQ ID NO: 91). In some embodiments, MICA consists of SEQ ID NO: 91. In some embodiments, MICA comprises an amino acid sequence with at least 85% identity to SEQ ID NO: 91 and which retains the ability to bind NKG2D. In some embodiments, MICA comprises an amino acid sequence with at least 85% identity to SEQ ID NO: 91 and which retains the ability to bind NKG2D and induce signaling by the bound receptor. In some embodiments, SEQ ID NO: 90 further comprises a 5′ ATG codon. In some embodiments, SEQ ID NO: 91 further comprises an N-terminal methionine residue.

In some embodiments, the therapeutic protein is UL16-binding protein 1 (ULBP1). In some embodiments, ULBP1 is encoded by a sequence comprising ggatgggtcgacacacactgtctttgctatgacttcatcatcactcctaagtccagacctgaaccacagtggtgtgaagttcaaggcc tggtggatgaaaggccttttcttcactatgactgtgttaaccacaaggccaaagcctttgcttctctggggaagaaagtcaatgtcaca aaaacctgggaagaacaaactgaaacactaagagacgtggtggatttccttaaagggcaactgcttgacattcaagtggagaattt aatacccattgagcccctcaccctgcaggccaggatgtcttgtgagcatgaagcccatggacacggcagaggatcttggcagttc ctcttcaatggacagaagttcctcctctttgactcaaacaacagaaagtggacagcacttcatcctggagccaagaagatgacaga gaagtgggagaagaacagggatgtgaccatgttcttccagaagatttcactgggggattgtaagatgtggcttgaagaatttttgatg tactgggaacaaatgctggatccaacaaaaccaccctctctggccccaggcacaacccaacccaaggccatggccaccaccctc agtccctggagccttctcatcatcttcctctgcttcattctagctggcaga (SEQ ID NO: 94). In some embodiments, ULBP1 is encoded by a sequence consisting of SEQ ID NO: 94. In some embodiments, ULBP1 is encoded by a sequence with at least 85% identity to SEQ ID NO: 94 and which encodes a protein with the ability to bind NKG2D. In some embodiments, ULBP1 is encoded by a sequence with at least 85% identity to SEQ ID NO: 94 and which encodes a protein with the ability to bind NKG2D and induce signaling by the engaged receptor. In some embodiments, ULBP1 comprises GWVDTHCLCYDFIITPKSRPEPQWCEVQGLVDERPFLHYDCVNHKAKAFASLGKK VNVTKTWEEQTETLRDVVDFLKGQLLDIQVENLIPIEPLTLQARMSCEHEAHGHG RGSWQFLFNGQKFLLFDSNNRKWTALHPGAKKMTEKWEKNRDVTMFFQKISLG DCKMWLEEFLMYWEQMLDPTKPPSLAPGTTQPKAMATTLSPWSLLIIFLCFILAGR (SEQ ID NO: 95). In some embodiments, ULBP1 consists of SEQ ID NO: 95. In some embodiments, ULBP1 comprises an amino acid sequence with at least 85% identity to SEQ ID NO: 95 and which retains the ability to bind NKG2D. In some embodiments, ULBP1 comprises an amino acid sequence with at least 85% identity to SEQ ID NO: 95 and which retains the ability to bind NKG2D and induce signaling by the bound receptor. In some embodiments, SEQ ID NO: 94 further comprises a 5′ ATG codon. In some embodiments, SEQ ID NO: 95 further comprises an N-terminal methionine residue.

In some embodiments, the therapeutic protein is Tumor necrosis factor ligand superfamily member 9 (TNFSF9/4-1BBL). In some embodiments, 4-1BBL is encoded by a sequence comprising atggaatacgcctctgacgcttcactggaccccgaagccccgtggcctcccgcgccccgcgctcgcgcctgccgcgtactgcctt gggccctggtcgcggggctgctgctgctgctgctgctcgctgccgcctgcgccgtcttcctcgcctgcccctgggccgtgtccgg ggctcgcgcctcgcccggctccgcggccagcccgagactccgcgagggtcccgagctttcgcccgacgatcccgccggcctct tggacctgcggcagggcatgtttgcgcagctggtggcccaaaatgttctgctgatcgatgggcccctgagctggtacagtgaccc aggcctggcaggcgtgtccctgacggggggcctgagctacaaagaggacacgaaggagctggtggtggccaaggctggagtc tactatgtcttctttcaactagagctgcggcgcgtggtggccggcgagggctcaggctccgtttcacttgcgctgcacctgcagcca ctgcgctctgctgctggggccgccgccctggctttgaccgtggacctgccacccgcctcctccgaggctcggaactcggccttcg gtttccagggccgcttgctgcacctgagtgccggccagcgcctgggcgtccatcttcacactgaggccagggcacgccatgcctg gcagcttacccagggcgccacagtcttgggactcttccgggtgacccccgaaatcccagccggactcccttcaccgaggtcgga a (SEQ ID NO: 98). In some embodiments, 4-1BBL is encoded by a sequence consisting of SEQ ID NO: 98. In some embodiments, 4-1BBL is encoded by a sequence with at least 85% identity to SEQ ID NO: 98 and which encodes a protein with the ability to bind 4-1BB. In some embodiments, 4-1BBL is encoded by a sequence with at least 85% identity to SEQ ID NO: 98 and which encodes a protein with the ability to bind 4-1BB and induce signaling by the engaged receptor. In some embodiments, 4-1BBL comprises MEYASDASLDPEAPWPPAPRARACRVLPWALVAGLLLLLLLAAACAVFLACPWA VSGARASPGSAASPRLREGPELSPDDPAGLLDLRQGMFAQLVAQNVLLIDGPLSW YSDPGLAGVSLTGGLSYKEDTKELVVAKAGVYYVFFQLELRRVVAGEGSGSVSL ALHLQPLRSAAGAAALALTVDLPPASSEARNSAFGFQGRLLHLSAGQRLGVHLHT EARARHAWQLTQGATVLGLFRVTPEIPAGLPSPRSE (SEQ ID NO: 99). In some embodiments, 4-1BBL consists of SEQ ID NO: 99. In some embodiments, 4-1BBL comprises an amino acid sequence with at least 85% identity to SEQ ID NO: 99 and which retains the ability to bind 4-1BB. In some embodiments, 4-1BBL comprises an amino acid sequence with at least 85% identity to SEQ ID NO: 99 and which retains the ability to bind 4-1BB and induce signaling by the bound receptor. In some embodiments, SEQ ID NO: 98 further comprises a 5′ ATG codon. In some embodiments, SEQ ID NO: 99 further comprises an N-terminal methionine residue.

In some embodiments, the therapeutic protein is Tumor necrosis factor ligand superfamily member 4 (TNFSF4/OX40L). In some embodiments, OX40L is encoded by a sequence comprising ctattgctggtggcctctgtaattcagggactggggctgctcctgtgcttcacctacatctgcctgcacttctctgctcttcaggtatcac atcggtatcctcgaattcaaagtatcaaagtacaatttaccgaatataagaaggagaaaggtttcatcctcacttcccaaaaggagga tgaaatcatgaaggtgcagaacaactcagtcatcatcaactgtgatgggttttatctcatctccctgaagggctacttctcccaggaag tcaacattagccttcattaccagaaggatgaggagcccctcttccaactgaagaaggtcaggtctgtcaactccttgatggtggcctc tctgacttacaaagacaaagtctacttgaatgtgaccactgacaatacctccctggatgacttccatgtgaatggcggagaactgatt cttatccatcaaaatcctggtgaattctgtgtcctt (SEQ ID NO: 100). In some embodiments, OX40L is encoded by a sequence consisting of SEQ ID NO: 100. In some embodiments, OX40L is encoded by a sequence with at least 85% identity to SEQ ID NO: 100 and which encodes a protein with the ability to bind OX40. In some embodiments, OX40L is encoded by a sequence with at least 85% identity to SEQ ID NO: 100 and which encodes a protein with the ability to bind OX40 and induce signaling by the engaged receptor. In some embodiments, OX40L comprises LLLVASVIQGLGLLLCFTYICLHFSALQVSHRYPRIQSIKVQFTEYKKEKGFILTSQK EDEIMKVQNNSVIINCDGFYLISLKGYFSQEVNISLHYQKDEEPLFQLKKVRSVNSL MVASLTYKDKVYLNVTTDNTSLDDFHVNGGELILIHQNPGEFCVL (SEQ ID NO: 101). In some embodiments, OX40L consists of SEQ ID NO: 101. In some embodiments, OX40L comprises an amino acid sequence with at least 85% identity to SEQ ID NO: 101 and which retains the ability to bind OX40. In some embodiments, OX40L comprises an amino acid sequence with at least 85% identity to SEQ ID NO: 101 and which retains the ability to bind OX40 and induce signaling by the bound receptor. In some embodiments, SEQ ID NO: 100 further comprises a 5′ ATG codon. In some embodiments, SEQ ID NO: 101 further comprises an N-terminal methionine residue.

It will be understood by a skilled artisan that therapeutic proteins that are to be secreted or displayed in the plasma membrane will require a signal peptide in order to ensure co-translational insertion into the ER. In some embodiments, the therapeutic protein comprises a signal peptide (SP). In some embodiments, the SP is an N-terminal SP. In some embodiments, the SP is the endogenous SP of the therapeutic protein. In some embodiments, the SP is a heterologous SP. In some embodiments, the SP is encoded by a nucleotide sequence comprising atgagaatttcgaaaccacatttgagaagtatttccatccagtgctacttgtgtttacttctaaacagtcattttctaactgaagct (SEQ ID NO: 55). In some embodiments, the SP is encoded by a nucleotide sequence consisting of SEQ ID NO: 55. In some embodiments, IL-15 comprises an SP encoded by SEQ ID NO: 55. In some embodiments, the SP comprises MRISKPHLRSISIQCYLCLLLNSHFLTEA (SEQ ID NO: 56). In some embodiments, the SP consists of SEQ ID NO: 56. In some embodiments, the SP is encoded by a nucleotide sequence comprising atggtattgggaaccatagatttgtgcagctgtttcagtgcagggcttcctaaaacagaa (SEQ ID NO: 72). In some embodiments, the SP is encoded by a nucleotide sequence consisting of SEQ ID NO: 72. In some embodiments, IL-15 comprises an SP encoded by SEQ ID NO: 72. In some embodiments, the SP comprises MVLGTIDLCSCFSAGLPKTE (SEQ ID NO: 73). In some embodiments, the SP consists of SEQ ID NO: 73. In some embodiments, the IL-15 SP is SEQ ID NO: 56. In some embodiments, the SP is encoded by a nucleotide sequence comprising atgtacaggatgcaactcctgtcttgcattgcactaagtcttgcacttgtcacaaacagt (SEQ ID NO: 57). In some embodiments, the SP is encoded by a nucleotide sequence consisting of SEQ ID NO: 57. In some embodiments, IL-2 comprises an SP encoded by SEQ ID NO: 57. In some embodiments, the SP comprises MYRMQLLSCIALSLALVTNS (SEQ ID NO: 58). In some embodiments, the SP consists of SEQ ID NO: 58. In some embodiments, the IL-2 SP is SEQ ID NO: 58. In some embodiments, the SP is encoded by a nucleotide sequence comprising atgcggctctgcatcccgcaggtgctgttggccttgttcctttccatgctgacagggccgggagaaggcagc (SEQ ID NO: 59). In some embodiments, the SP is encoded by a nucleotide sequence consisting of SEQ ID NO: 59. In some embodiments, the scFv comprises an SP encoded by SEQ ID NO: 59. In some embodiments, the SP comprises MRLCIPQVLLALFLSMLTGPGEGS (SEQ ID NO: 60). In some embodiments, the SP consists of SEQ ID NO: 60. In some embodiments, the scFv SP is SEQ ID NO: 60. In some embodiments, the SP is encoded by a nucleotide sequence comprising atgggagttaaggtattgttcgcattgatttgcatagccgtggccgaagct (SEQ ID NO: 84). In some embodiments, the SP is encoded by a nucleotide sequence consisting of SEQ ID NO: 84. In some embodiments, the scFv comprises an SP encoded by SEQ ID NO: 84. In some embodiments, the SP comprises MGVKVLFALICIAVAEA (SEQ ID NO: 85). In some embodiments, the SP consists of SEQ ID NO: 85. In some embodiments, the scFv SP is SEQ ID NO: 85. In some embodiments, the SP consists of SEQ ID NO: 69. In some embodiments, the CD40L SP is SEQ ID NO: 69. In some embodiments, the SP is encoded by a nucleotide sequence comprising atgtggctgcagagcctgctgctcttgggcactgtggcctgtagcatctct (SEQ ID NO: 76). In some embodiments, the SP is encoded by a nucleotide sequence consisting of SEQ ID NO: 76. In some embodiments, GM-CSF comprises an SP encoded by SEQ ID NO: 76. In some embodiments, the SP comprises MWLQSLLLLGTVACSIS (SEQ ID NO: 77). In some embodiments, the SP consists of SEQ ID NO: 77. In some embodiments, the GM-CSF SP is SEQ ID NO: 77. In some embodiments, the SP is encoded by a nucleotide sequence comprising atgacagtgctggcgccagcctggagcccaacaacctatctcctcctgctgctgctgctgagctcgggactcagtggg (SEQ ID NO: 80). In some embodiments, the SP is encoded by a nucleotide sequence consisting of SEQ ID NO: 80. In some embodiments, FLT3LG comprises an SP encoded by SEQ ID NO: 80. In some embodiments, the SP comprises MTVLAPAWSPTTYLLLLLLLSSGLSG (SEQ ID NO: 81). In some embodiments, the SP consists of SEQ ID NO: 81. In some embodiments, the FLT3LG SP is SEQ ID NO: 81. In some embodiments, the SP is encoded by a nucleotide sequence comprising atggctcaatccttggcgctgtccttgctgatcttggtgctggcatttggcattccccgcacacaagga (SEQ ID NO: 88). In some embodiments, the SP is encoded by a nucleotide sequence consisting of SEQ ID NO: 88. In some embodiments, CCL21 comprises an SP encoded by SEQ ID NO: 88. In some embodiments, the SP comprises MAQSLALSLLILVLAFGIPRTQG (SEQ ID NO: 89). In some embodiments, the SP consists of SEQ ID NO: 89. In some embodiments, the CCL21 SP is SEQ ID NO: 89. In some embodiments, the SP is encoded by a nucleotide sequence comprising atggggctgggcccggtcttcctgcttctggctggcatcttcccttttgcacctccgggagctgctgctgag (SEQ ID NO: 92). In some embodiments, the SP is encoded by a nucleotide sequence consisting of SEQ ID NO: 92. In some embodiments, MICA comprises an SP encoded by SEQ ID NO: 92. In some embodiments, the SP comprises MGLGPVFLLLAGIFPFAPPGAAAE (SEQ ID NO: 93). In some embodiments, the SP consists of SEQ ID NO: 93. In some embodiments, the MICA SP is SEQ ID NO: 93. In some embodiments, the SP is encoded by a nucleotide sequence comprising atggcagcggccgccagccccgcgttccttctgtgcctcccgcttctgcacctgctgtctggctggtcccgggca (SEQ ID NO: 96). In some embodiments, the SP is encoded by a nucleotide sequence consisting of SEQ ID NO: 96. In some embodiments, ULBP1 comprises an SP encoded by SEQ ID NO: 96. In some embodiments, the SP comprises MAAAASPAFLLCLPLLHLLSGWSRA (SEQ ID NO: 97). In some embodiments, the SP consists of SEQ ID NO: 97. In some embodiments, the ULBP1 SP is SEQ ID NO: 97. In some embodiments, the SP is encoded by a nucleotide sequence comprising atggaaagggtccaacccctggaagagaatgtgggaaatgcagccaggccaagattcgagaggaacaag (SEQ ID NO: 102). In some embodiments, the SP is encoded by a nucleotide sequence consisting of SEQ ID NO: 102. In some embodiments, OX40L comprises an SP encoded by SEQ ID NO: 102. In some embodiments, the SP comprises MERVQPLEENVGNAARPRFERNK (SEQ ID NO: 103). In some embodiments, the SP consists of SEQ ID NO: 103. In some embodiments, the OX40L SP is SEQ ID NO: 103.

It will be understood that any open reading frame described herein may also comprise a 3′ stop codon. Any stope codon may be used including TGA, TAA and TAG. If the nucleic acid molecule is an RNA, then an RNA stop codon such as UGA, UAA, UAG will be used. In general, herein, DNA sequences are given, but it will be understood that the equivalent RNA sequences with all thymidines replaced by uracils are envisioned.

In some embodiments, the protein is a reporter protein. In some embodiments, the output is a reporter molecule. In some embodiments, the reporter molecule is a reporter protein. In some embodiments, the reporter is fluorescent. In some embodiments, the reporter is a detectable molecule. In some embodiments, the reporter is an enzyme.

In some embodiments, the system further comprises a fourth nucleic acid molecule comprising a second polynucleotide operably linked to the third transcriptional regulatory element. In some embodiments, the polynucleotide of the third nucleic acid molecule is a first polynucleotide. In some embodiments, the fourth nucleic acid construct is a second output construct. In some embodiments, the system comprises at least one output construct. In some embodiments, an output construct comprises a polynucleotide operably linked to the third transcriptional regulatory element. In some embodiments, the system comprises a plurality of output constructs. In some embodiments, the system comprises at least 1, 2, 3, 4, 5, 6, 7, 8, 9 or 10 output constructs. Each possibility represents a separate embodiment of the invention. In some embodiments, the system comprises three output constructs. In some embodiments, each output construct produces the same polynucleotide. In some embodiments, each output construct produces a different polynucleotide. In some embodiments, a plurality of output molecules produces a therapeutic complex. In some embodiments, the complex is an antibody. In some embodiments, the complex is an activation complex. In some embodiments, the complex is a regulatory complex. In some embodiments, the complex is a lethal complex. In some embodiments, the complex is an apoptotic complex. In some embodiments, the complex is cytotoxic. In some embodiments, the complex is immunomodulatory. In some embodiments, all the outputs are immunomodulatory. In some embodiments, all the outputs are cytotoxic. In some embodiments, all the outputs are anticancer.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the therapeutic molecule is an antibody. In some embodiments, the immunotherapy is an antibody. In some embodiments, the therapeutic molecule is an antigen binding domain. In some embodiments, the immunotherapy is an antigen binding domain. In some embodiments, the therapeutic molecule is a single chain antibody. In some embodiments, the immunotherapy is a single chain antibody. In some embodiments, the therapeutic molecule is a single domain antibody. In some embodiments, the immunotherapy is a single domain antibody. In some embodiments, the antigen binding fragment is a single chain antibody. In some embodiments, antigen binding fragment is a single domain antibody. In some embodiments, the antibody comprises a transmembrane domain. In some embodiments, the antibody is a cell surface antibody. In some embodiments, the antibody is a surface-displayed antibody.

As used herein, the terms “single chain antibodies” and “single chain variable fragments” are used synonymously and refer to a fusion protein of variable region of heavy and light chains of immunoglobulins, connected by a short peptide linker. As used herein, the terms “single domain antibody”, “nanobody”, “DARPin” and “VHH” are used synonymously and refer to an antibody fragment consisting of a single monomeric variable antibody domain.

Other polypeptides which may be encoded by the polynucleotide include antigen-binding antibody fragments such as single domain antibodies (“dAbs”), Fv, scFv, Fab′ and CHI and CK or CL domain has been excised. As minibodies are smaller than conventional antibodies they should achieve better tissue penetration in clinical/diagnostic use but being bivalent they should retain higher binding affinity than monovalent antibody fragments, such as dAbs. Accordingly, unless the context dictates otherwise, the term “antibody” as used herein encompasses not only whole antibody molecules, but also antigen-binding antibody fragments of the type discussed above. Each framework region present in the encoded polypeptide may comprise at least one amino acid substitution relative to the corresponding human acceptor framework. Thus, for example, the framework regions may comprise, in total, three, four, five, six, seven, eight, nine, ten, eleven, twelve, thirteen, fourteen, or fifteen amino acid substitutions relative to the acceptor framework regions. Given the properties of the individual amino acids comprising the disclosed protein products, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

In some embodiments, at least one of the first, second and third nucleic acid construct comprises a tag. In some embodiments, the first construct comprises a tag. In some embodiments, the second construct comprises a tag. In some embodiments, the tag is a degradation tag. In some embodiments, the tag is a detectable tag. In some embodiments, the tag is a fluorescent tag. In some embodiments, the tag is a protein. In some embodiments, the tag is in frame with the fusion protein. In some embodiments, the tag is separated from the fusion protein by a cleavable linker. In some embodiments, the linker is a peptide. In some embodiments, the cleavable peptide is a viral peptide. In some embodiments, the viral peptide is the P2A peptide.

In some embodiments, the nucleic acid molecule system is configured to express the polynucleotide in a cancer cell. In some embodiments, express is specifically express. In some embodiments, the cancer is colorectal cancer and the first nucleic acid molecule comprises binding sites for a first TFs selected from LEF1, SOX9, GATA10 and TCF7L1 and the second nucleic acid molecule comprises binding sites for a second TF selected from LEF1, SOX9, GATA10 and TCF7L1 wherein the first and second TF are different TFs. In some embodiments, the cancer is breast cancer and the first nucleic acid molecule comprises binding sites for a first TFs selected from SOX9, MYB and MYBL2 and the second nucleic acid molecule comprises binding sites for a second TF selected from SOX9, MYB and MYBL2 wherein the first and second TF are different TFs. In some embodiments, the cancer is breast cancer and the first nucleic acid molecule comprises binding sites MYB and the second nucleic acid molecule comprises binding sites for MYBL2.

In some embodiments, the cancer is a colorectal cancer and the first nucleic acid molecule comprises binding sites for LEF1 and the second nucleic acid molecule comprises binding sites for a second TF selected from SOX9 and TCF7L1. In some embodiments, the first nucleic acid molecule comprises repeats of SEQ ID NO: 5 and the second nucleic acid molecule comprises repeats of SEQ ID NO: 1. In some embodiments, the first nucleic acid molecule comprises SEQ ID NO: 12 and the second nucleic acid molecule comprises SEQ ID NO: 8. In some embodiments, the first nucleic acid molecule comprises repeats of SEQ ID NO: 4 and the second nucleic acid molecule comprises repeats of SEQ ID NO: 3. In some embodiments, the first nucleic acid molecule comprises SEQ ID NO: 11 and the second nucleic acid molecule comprises SEQ ID NO: 10. In some embodiments, nucleic acid molecule system is for use in expressing the polynucleotide in a colorectal cancer cell. In some embodiments, the cell is in a subject.

In some embodiments, the cancer is breast cancer and the first nucleic acid molecule comprises binding sites for MYBL2 and the second nucleic acid molecule comprises binding sites for MYB. In some embodiments, the first nucleic acid molecule comprises repeats of SEQ ID NO: 6 and the second nucleic acid molecule comprises repeats of SEQ ID NO: 7. In some embodiments, the first nucleic acid molecule comprises SEQ ID NO: 13 and the second nucleic acid molecule comprises SEQ ID NO: 14. In some embodiments, nucleic acid molecule system is for use in expressing the polynucleotide in a breast cancer cell. In some embodiments, the cell is in a subject.

In some embodiments, the third nucleic acid molecule comprises a sequence comprising SEQ ID NO: 63. In some embodiments, the third nucleic acid molecule comprises a sequence consisting of SEQ ID NO: 63. In some embodiments, the third nucleic acid molecule comprises a sequence comprising SEQ ID NO: 64. In some embodiments, the third nucleic acid molecule comprises a sequence consisting of SEQ ID NO: 64. In some embodiments, the third nucleic acid molecule comprises a sequence comprising SEQ ID NO: 65. In some embodiments, the third nucleic acid molecule comprises a sequence consisting of SEQ ID NO: 65. In some embodiments, the system comprises SEQ ID NO: 63, 64 and 65. In some embodiments, the system comprises a third nucleic acid molecule comprising SEQ ID NO: 63, a fourth nucleic acid molecule comprising SEQ ID NO: 64 and a fifth nucleic acid molecule comprising SEQ ID NO: 65.

By another aspect, there is provided a composition comprising a nucleic acid molecule of the invention.

By another aspect, there is provided a composition comprising a nucleic acid molecule system of the invention.

In some embodiments, the composition is a pharmaceutical composition. In some embodiments, the composition is a diagnostic composition. In some embodiments, the composition is formulated for systemic administration. In some embodiments, the composition is formulated for intratumoral administration. In some embodiments, the composition is formulated for intravenous administration. In some embodiments, the composition is formulated for administration to a subject. In some embodiments, the composition is formulated for local administration.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, intratumoral, or intraperitoneal.

The dosage administered will be dependent upon the age, health, and weight of the recipient, kind of concurrent treatment, if any, frequency of treatment, and the nature of the effect desired.

In some embodiments, the subject is a mammal. In some embodiments, the subject is a human. In some embodiments, the subject is need of the composition of the invention. In some embodiments, the subject is need of treatment with a composition of the invention. In some embodiments, the subject is in need of a method of the invention. In some embodiments, the subject is in need of expression of the polynucleotide. In some embodiments, the subject suffers from cancer. In some embodiments, the subject suffers from breast cancer. In some embodiments, the subject suffers from colorectal cancer.

In some embodiments, the composition further comprises a pharmaceutically acceptable carrier excipient or adjuvant. In some embodiments, the composition is a therapeutic composition. In some embodiments, the composition comprises a therapeutically effective amount of the nucleic acid molecule system of the invention. In some embodiments, a therapeutically effective amount of the nucleic acid molecule system is an amount sufficient to produce a therapeutically effective amount of the polynucleotide. In some embodiments, a therapeutically effective amount of the polynucleotide is a therapeutically effective amount of the polynucleotide within a target cell. In some embodiments, effective is effective to treat the cancer.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, gel, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

By another aspect, there is provided a method of treating cancer in a subject in need thereof, the method comprising administering to the subject a nucleic acid molecule of the invention, a nucleic acid molecule system of the invention or a composition of the invention, thereby treating cancer.

By another aspect, there is provided a method of detecting a cancer cell in a subject, the method comprising administering a nucleic acid molecule of the invention, a nucleic acid molecule system of the invention or a composition of the invention to the subject and detecting expression of the polynucleotide, thereby detecting a cancer cell in a subject.

In some embodiments, the cancer is colorectal cancer and a nucleic acid molecule comprises a binding site for SOX9, GATA10, LEF1 and/or TCF7L1. In some embodiments, the cancer is breast cancer and a nucleic acid molecule comprises a binding site for SOX9, MYB and/or MYBL2. In some embodiments, the cancer is breast cancer and a nucleic acid molecule comprises a binding site for MYB and/or MYBL2.

In some embodiments, the polynucleotide is a detectable molecule. In some embodiments, the polynucleotide encodes a detectable molecule. In some embodiments, the molecule is a protein. In some embodiments, the molecule is an RNA. In some embodiments, detectable is fluorescent. In some embodiments, the detectable molecule is a reporter protein. In some embodiments, the molecule is a barcode. In some embodiments, the barcode is a nucleic acid barcode. As used here, the term “barcode”, and “nucleic acid barcode”, are interchangeable with each other and have the same meaning. The nucleic acid molecule serving as a barcode is a polymer of nucleic acids and may be single-stranded or double-stranded, optionally containing synthetic, non-natural or altered nucleotide bases. In some embodiments, the nucleic acid molecule is labelled, for instance, with biotin, a radiolabel, or a fluorescent label.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Transfection and infection of cells: 6×10\hat{ }5 HEK293T cells were plated per well in a 6-well dish one day before transfection/or on the day of the infection. For transfection, a DNA mix (2 ug total) was prepared containing 0.5 ug packaging and envelope plasmids and 1 ug viral plasmid. Optimem and FuGENE® HD Transfection Reagent were added according to manufacturer's instructions and incubated for 10 minutes at room temperature. Mixture was added dropwise to cells and removed 18 hours later to be replaced by fresh media. For infection, virus was added directly to the cells followed by the addition of polybrene. The cells were incubated with the virus for 24 hours followed by removal and replacement with fresh media.

Flow cytometry: Flow cytometry for fluorescent reporter genes expression or antibodies staining was performed according to standard protocols. The following anti-human antibodies were used: anti-CD25-PE (Miltenyi Biotec), anti-CD69-APC (Miltenyi Biotec), anti-CD45-APC (Miltenyi Biotec), anti-CD3-VioBlue (Miltenyi Biotec), anti-CD8-PE (Miltenyi Biotec), anti-CD56-APC (Miltenyi Biotec), anti-CD16-PE (Miltenyi Biotec), anti-CD4-FITC (Miltenyi Biotec), anti-CD154-APC (Miltenyi Biotec), anti-CD19-PC (Miltenyi Biotec), and anti-CD14-APC (Miltenyi Biotec). The following control antibodies were also used anti-HA-PE (Miltenyi Biotec), REA Control Antibody (S) human IgG1-APC (Miltenyi Biotec), REA Control Antibody (S) human IgG1-PE (Miltenyi Biotec), REA Control Antibody (S) human IgG1-VioBlue (Miltenyi Biotec), and REA Control Antibody (S) human IgG1-FITC (Miltenyi Biotec). LIVE/DEAD fixable violet dead stain kit (Invitrogen) was used for live/dead staining.

Measuring serum and culture media cytokine levels: Serum or culture media levels of IL-2 and IFN-g were determined using the ELISA MAX™ Deluxe Set Human IL-2 kit (BioLegend) and the ELISA MAX™ Deluxe Set Human IFN-g kit (BioLegend). ELISAs were performed according to the manufacturer's protocol.

### Example 1: Determining Colorectal Cancer Specific Synthetic Promoters

A novel RNA-barcode-based promoter library containing 6,000 synthetic promoters each containing a different transcription factor (TF) binding site (BS) was established. Each synthetic promoter contained 3-12 repeats of a TF-BS found in the literature or predicted by computer modeling. Downstream of the promoter, each construct contained a unique 20 bp RNA barcode that could be used to determine unique mRNA expression from that synthetic promoter.

The library was screened to identify potential colorectal cancer (CRC)-specific synthetic promoters and breast cancer (BC) specific synthetic promoters. To facilitate this screening, an initial screen was carried out on 5 colorectal cancer cells, 5 breast cancer cells, and 5 different non-tumorigenic cell lines from various tissues, used as negative controls. The library was transfected into the cell lines and next generation sequencing (NGS) was carried out to measure barcode expression (as a proxy for promoter activity). The NGS results yield 24 potential BC-specific promoters, and 31 potential CRC-specific promoters. Based on this analysis, the 5 best synthetic promoters for CRC were selected for further analysis, and the 4 best for BC.

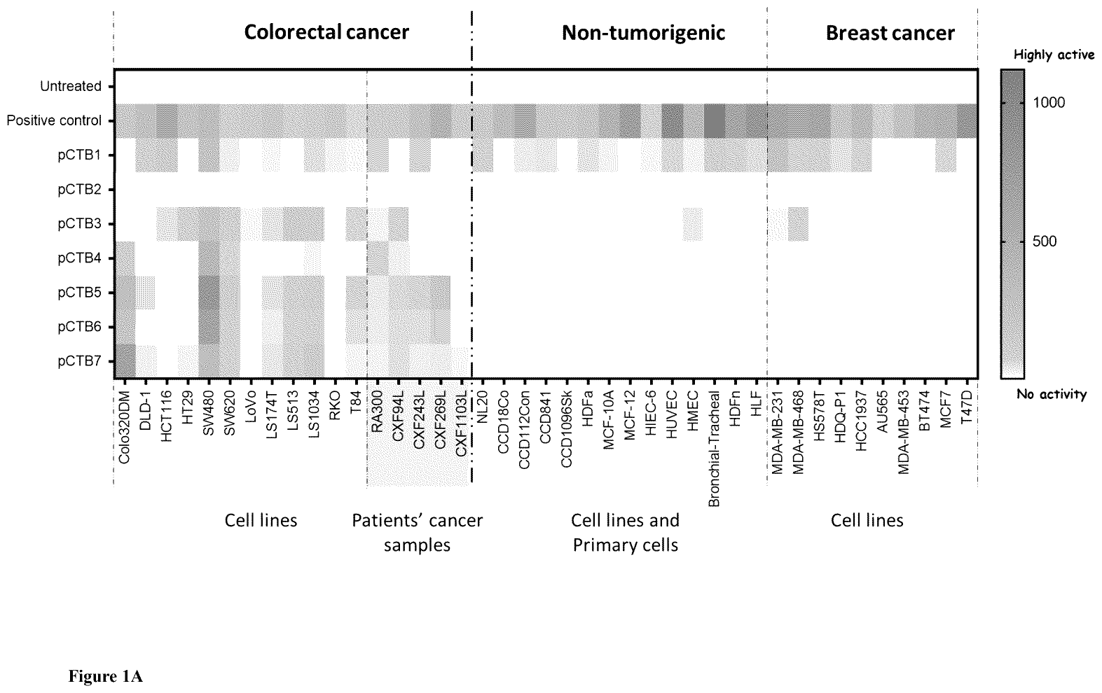

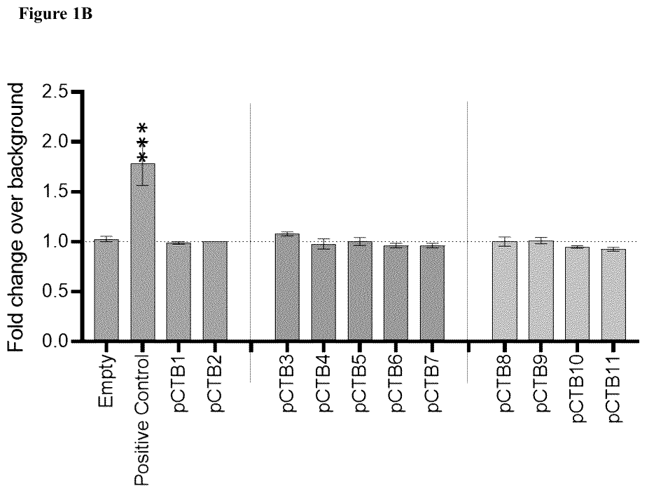

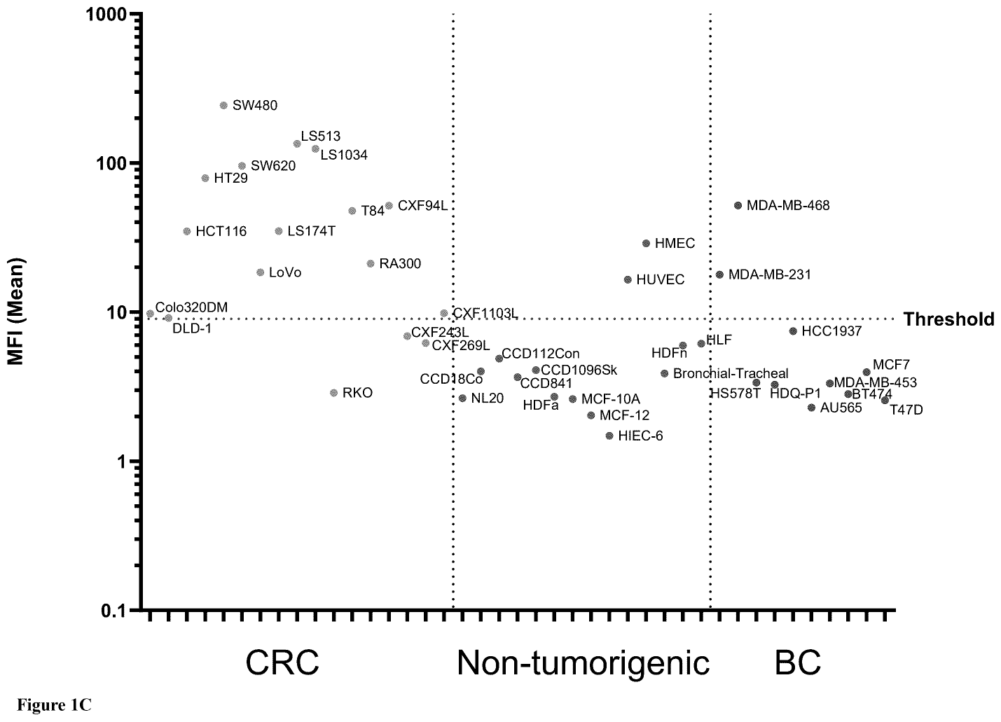

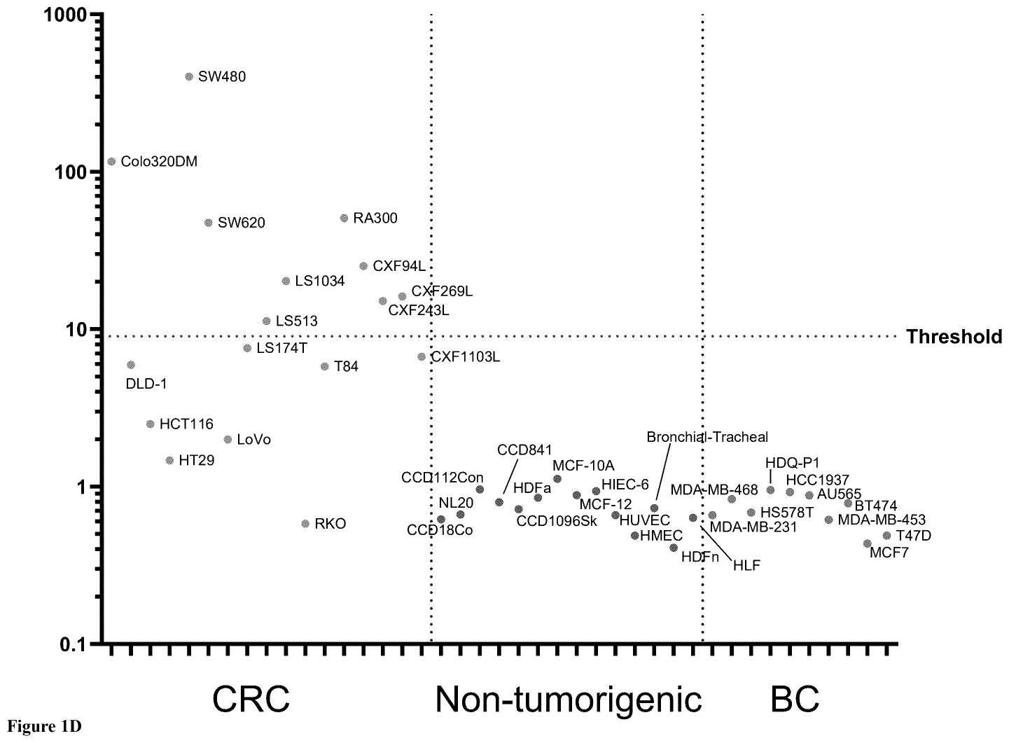

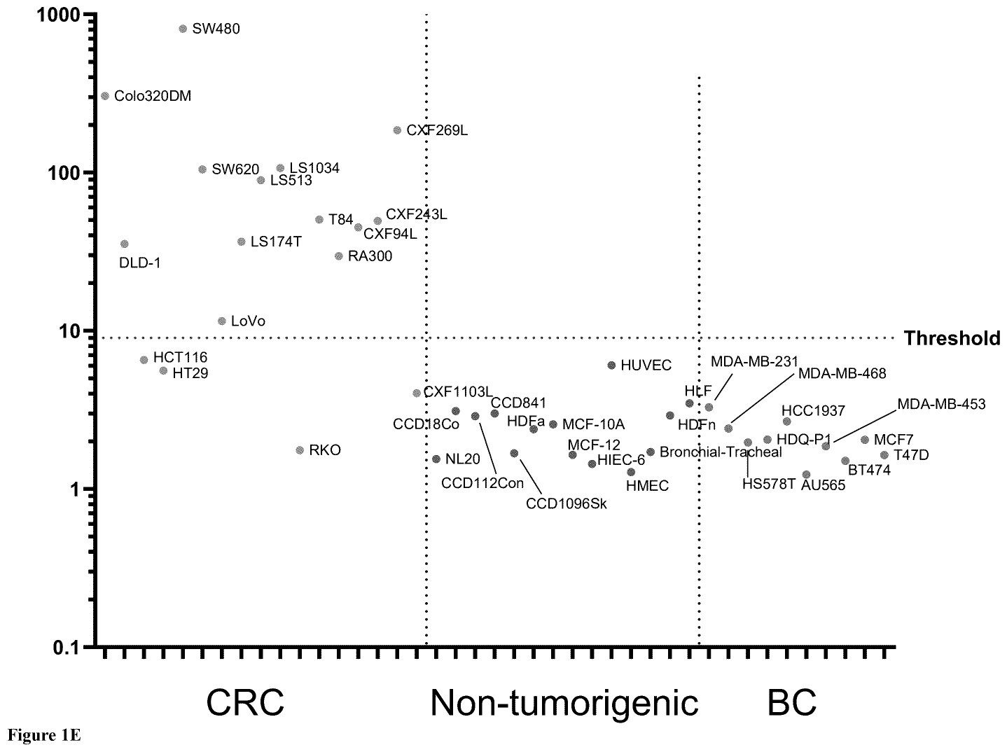

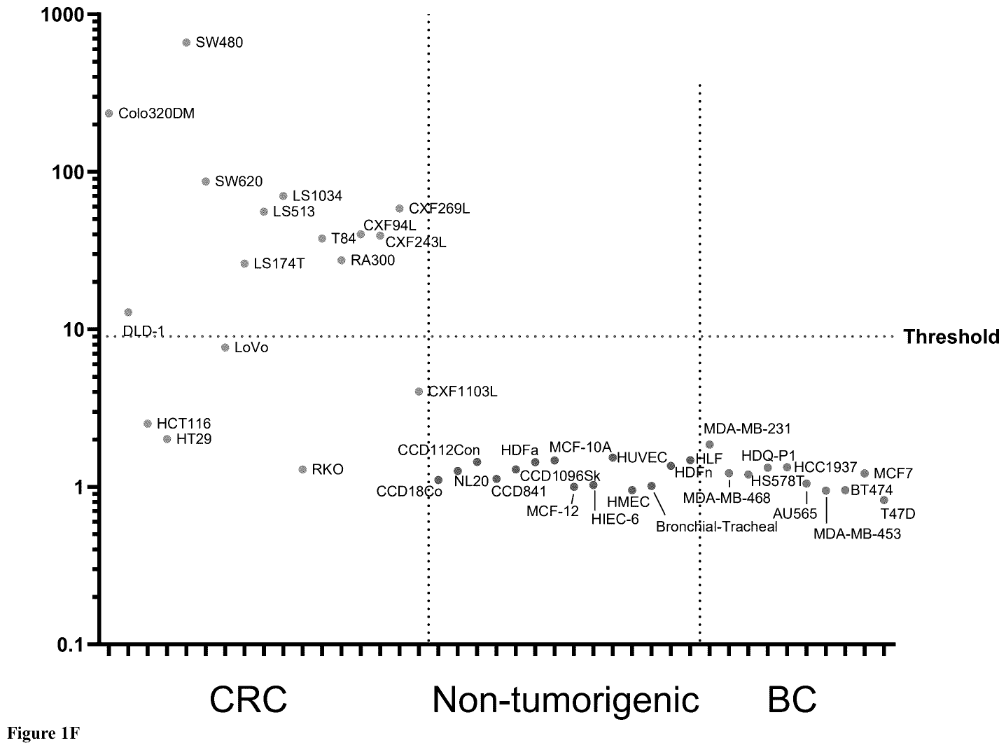

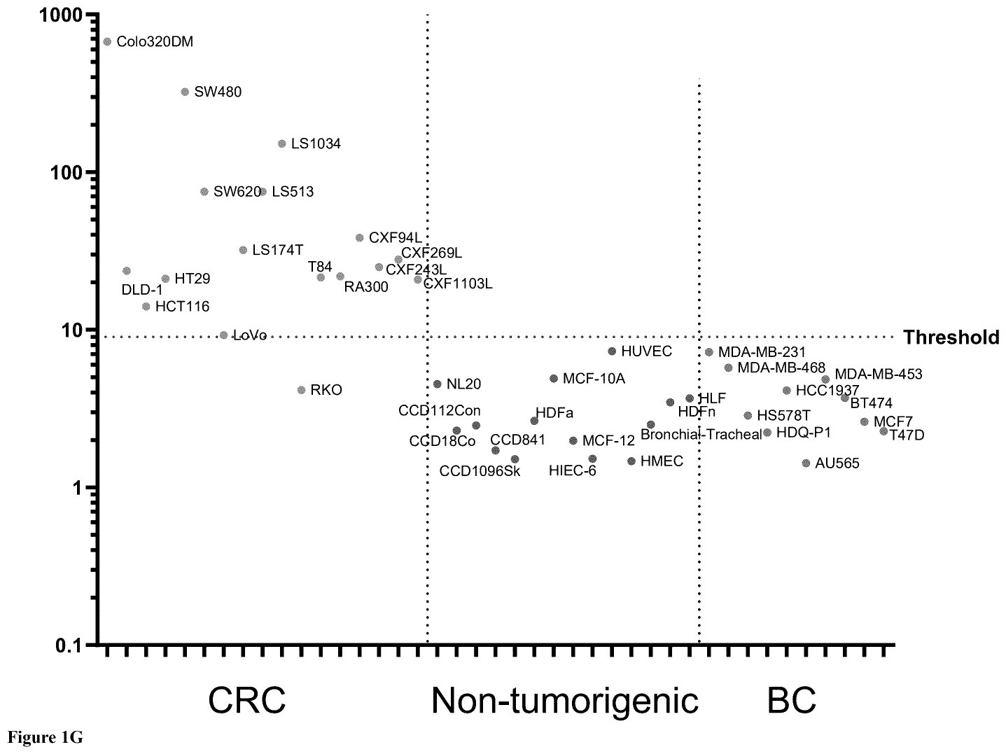

For the identification of CRC-specific promoters, each selected synthetic promoter was further tested for specificity using a promoter-EGFP cell-based assay. Each of the five synthetic promoter candidates was cloned into a lentiviral plasmid backbone upstream to the EGFP sequence. Virus was produced and was transduced into 12 CRC cell lines, 5 CRC patient samples, 10 breast cancer cell lines and 14 non-tumorigenic cells, followed by measurement of EGFP output levels using flow cytometry was carried out (FIG. 1A). Empty vector was used as a negative control along with pCTB2, a non-active promoter, to determine background levels. A lentiviral plasmid with EGFP downstream of the constitutive promoter UbC was used as a positive control (called PC-positive control). pCTB1 which contains a non-specific promoter was also used as a positive control. EGFP protein output expression was measured 96 hours post-infection by flow cytometry. pCTB3-7 all showed high expression in many of the CRC cell lines and patient samples and no expression in most of the non-tumorigenic cells. Additionally, pCTB3 showed mild expression in the two triple negative breast cancer cell lines, MDA-MB-231 and MDA-MB-468. PBMCs from healthy donors were also tested and expression was not observed (FIG. 1B). A cutoff for positive expression (MFI 9) was determined (FIG. 1C-1G) and was found to be useful for all 5 promoters. All five CRC-specific promoter candidates showed expression in the vast majority of CRC cell lines and expression in a maximum of two non-tumorigenic cell lines. Four of the promoters (other than pCTB3) showed no expression above the threshold in any of the non-tumorigenic cell lines. Therefore, it was determined that these synthetic promoters are indeed CRC-specific. The individual TF-BSs present in each syntenic promoter as well as the full sequences of the TF-BS regions are summarized in Table 1.

TABLE 1
[]synthetic promoter sequences; CRC-specific

| Synthetic | | | Promoter |
|---|---|---|---|
| promoter | TF | BS (SEQ ID NO:) | region |
| pCTB3 | SOX9 | GAACAAAGGCCCCTTT | SEQ ID NO: 8 |
| | | (1) | |
| pCTB4 | GATA10 | TCCCTTTTATCT (2) | SEQ ID NO: 9 |
| pCTB5 | TCF7L1 | TGCCTTTGATGTT (3) | SEQ ID NO: 10 |
| pCTB6 | LEF1 | AAAGATCAAAGGGTT (4) | SEQ ID NO: 11 |
| pCTB7 | LEF1 | TCCTTTGATTTGCT (5) | SEQ ID NO: 12 |

### Example 2: Designing AND Circuits for Highly Specific CRC Expression

An AND gate genetic circuit is one that requires two inputs to both be present (input 1 AND input 2) for the circuit to be active. Such circuits can greatly enhance specificity as even if one input is active in off target cells the circuit would not be active so long as the other input did not share the same off target expression. It was thus hypothesized that an AND circuit that made use of two of the CRC-specific synthetic promoters would have enhanced specificity and reduced off-target effects. Such a dual-promoter integrator (DPI) for producing cancer cell specificity was previously described in Nissim and Bar-Ziv, “A tunable dual-promoter integrator for targeting of cancer cells”, Mol. Syst. Biol., 2010 Dec. 21; 6:444, herein incorporated by reference in its entirety.

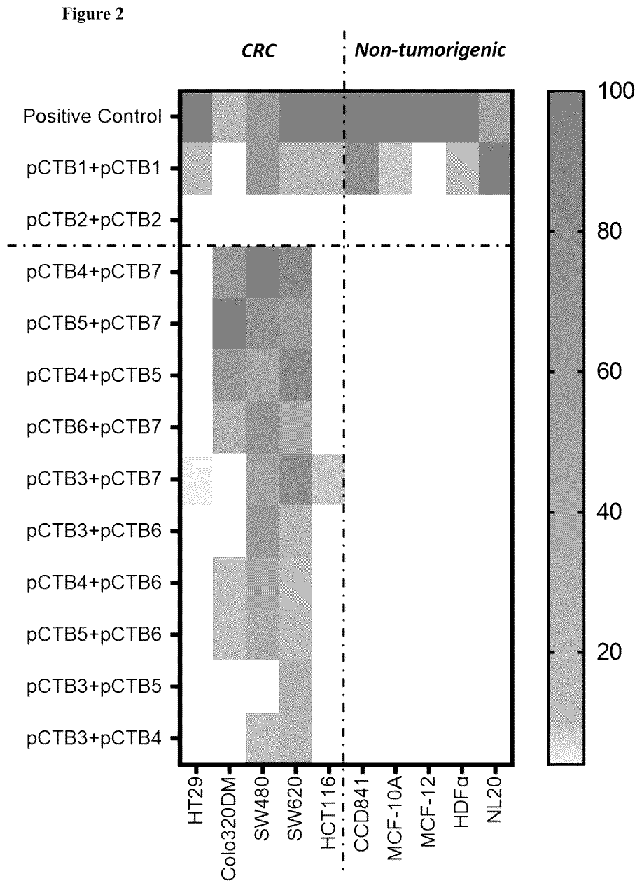

This system was used to drive highly specific CRC expression of EGFP. A first synthetic promoter driving expression of Gal4BD-Coh2 (SEQ ID NO: 16; a fusion protein of the Coh2 and the Gal4 binding domain) and a second promoter driving expression of VP16AD-DocS (SEQ ID NO: 15; a fusion protein of DocS and the VP16 activation domain) were used as the inputs. The output molecule contained Gal4-binding sites (CGGAGTACTGTCCTCCG; SEQ ID NO: 17) operatively linked to a minimal promoter driving the expression of EGFP (SEQ ID NO: 19). Bases −38 to +6 of the adenovirus major late promoter (GGGGGGCTATAAAAGGGGGTGGGGGCGTTCGTCCTCACTCT; SEQ ID NO: 18) were used as the minimal promoter and the constructs were inserted into a lentiviral vector for expression in target cells. EGFP was measured 96 hours post-infection by flow cytometry. Once again various CRC cell lines and non-tumorigenic cell lines were tested. Combinations of the 5 CRC-specific promoters were tested and it was determined that all expression in non-cancerous tissues was abolished, while high expression was achieved in the various cancerous cell lines (FIG. 2). Two specific combinations were found to be particularly useful. Combination of pCTB3 and pCTB7 produced the best expression in HT29 cells and HCT116 cells, two moderately differentiated cell lines. Combination of pCTB5 and pCTB6 produced strong expression in Colo320DM, SW480 and SW620 cells, the latter two of which are metastatic, poorly differentiated cell lines. The combination of pCTB3 and pCTB7 was appealing as a number of cell lines and patient samples had shown these two promoters to have the strongest expression (see FIG. 1A).

### Example 3: Selecting Output Molecules

The transcript produced by the output construct is meant to induce CRC killing. Therefore, the output construct can encode a toxin, cell death protein or the like. However, there is a risk that not all of the patient's tumor cells will be infected with the circuit and thus die due to production of the output protein. As such, output proteins were investigated that impact the tumor microenvironment (TME) and thus produce a cytotoxic effect against the whole tumor and not just the cells that express the circuit. To this end immunomodulators, which can enhance the immune response against the tumor as a whole, were investigated. Three main functional groups were examined: molecules able to modulate an immunosuppressive TME into an immunogenic one; chemoattractants of antigen presenting cells (APCs), cytotoxic T lymphocytes (CTLs) and natural killer (NK) cells; and CTL and NK cell activators. Further, the DPI circuit allows for easy combination of different types of immunomodulators. The circuit can simply be expanded to have more than one output construct or to have a single construct that encodes multiple proteins.

Four sets of output combinations were tested and are summarized in Table 2. Each combination includes molecules from at least two of the functional groups.

TABLE 2
Four immunomodulator combinations

| Combinations | Outputs |
|---|---|
| Combination 1 | IL-2, IL-15, anti-CD3e |
| Combination 2 | IL-2, IL-15, 4-1BBL |
| Combination 3 | IL-2, CD80, CD86 |
| Combination 4 | IL-15, anti-CD3e, CXCL10 |

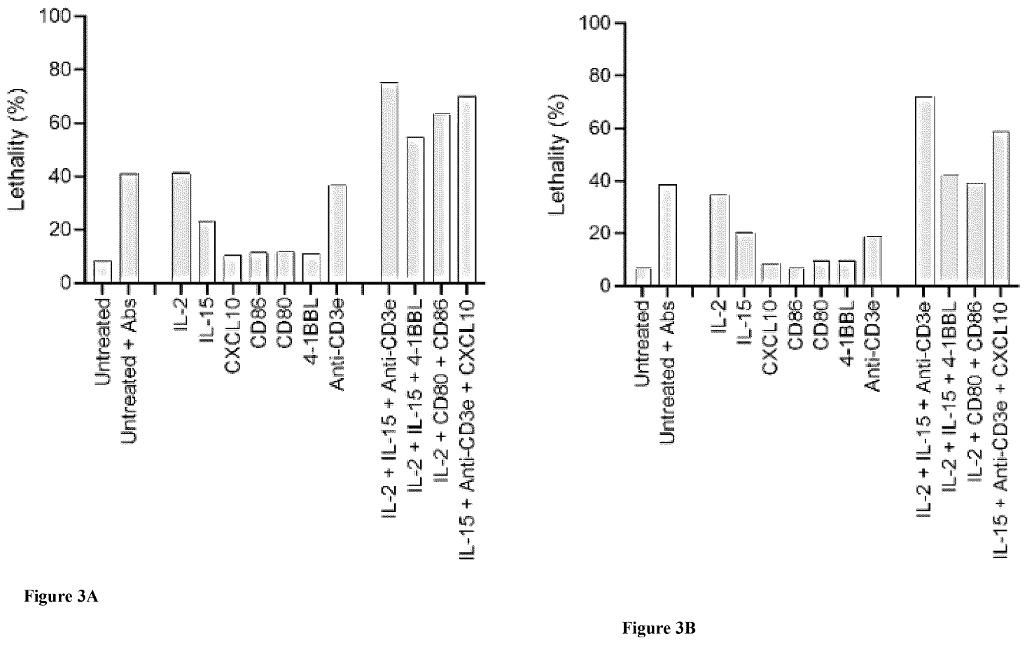

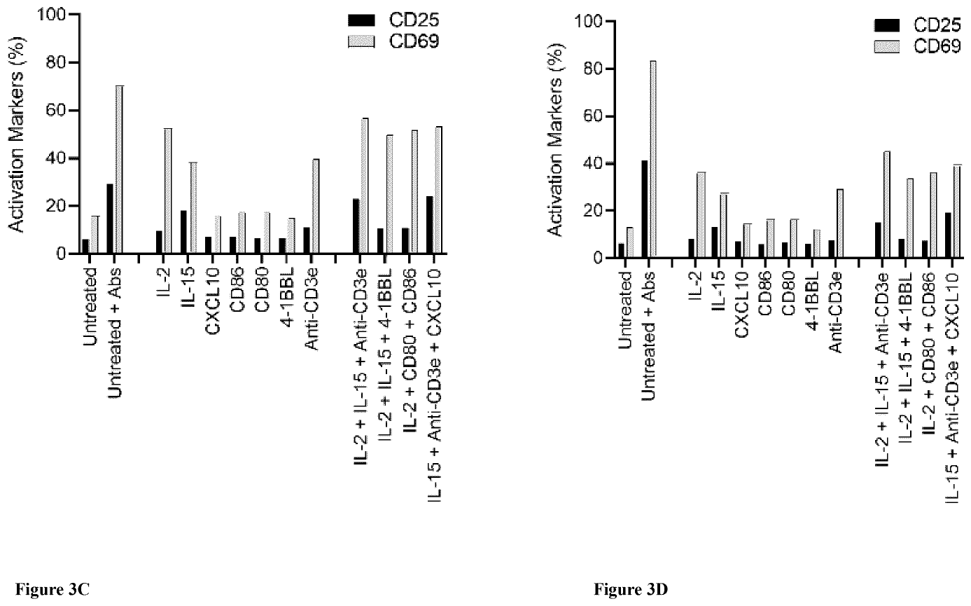

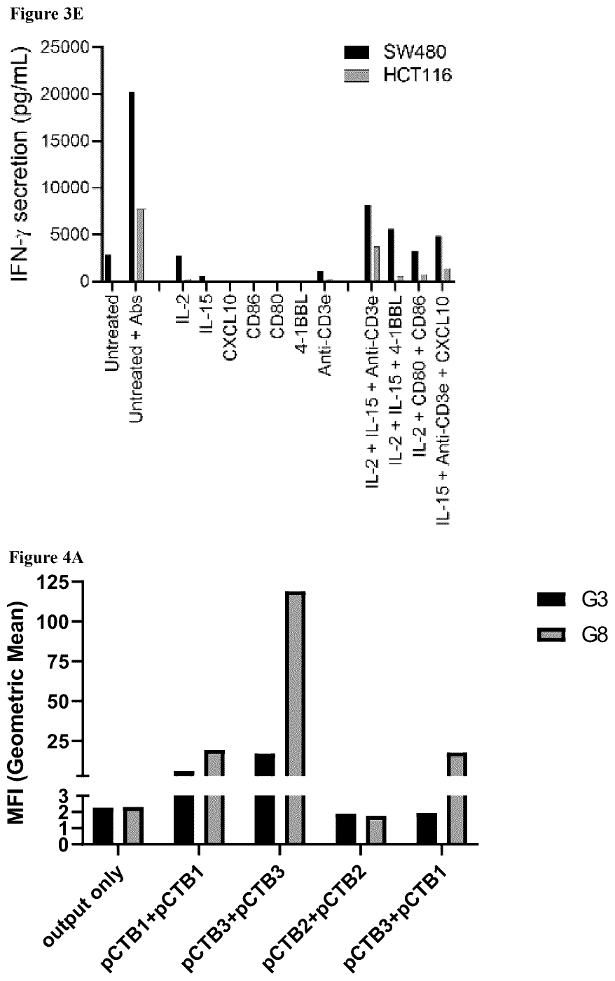

To facilitate the selection process, the various immunomodulatory genes were cloned into an expression lentivirus vector under the control of the UbC constitutive promoter. A co-culture cytotoxicity cell-based assay was used to test the efficacy of the various molecules alone and in combination in several CRC cell lines. The assay consists of co-culturing CRC cells together with naïve immune cell PBMCs and monitoring cell death. Live/Dead cell viability dye staining followed by flow cytometry was used to quantify CRC cell death. Immune cell activation was detected by measuring levels of IFN-g secretion by standard ELISA and detection of cell surface activation markers CD25 and CD69 by flow cytometry. HCT116 and SW480 cells were infected with the various proteins/molecules, alone or in combination, and then co-cultured with naïve PBMCs obtained from healthy donor at an effector cell to target cell ratio of 10:1 (PBMCs: CRC cells). 48 hours after co-culture the immune cells were tested for activation markers, the culture media was tested for cytokine secretion, and CRC cell viability was measured. CRC cells mock administered the immunomodulatory proteins were used as a negative control. Co-cultures of uninfected CRC cells and immune cells activated by anti-CD3 and anti-CD28 antibodies were used as a positive control. IL-2, IL-15 and anti-CD3e showed greater individual killing ability over the other tested molecules in HCT116 cells (FIG. 3A) and SW480 cells (FIG. 3B). It was thus not surprising that the combination of these three molecules (Combination 1) was the most effective combination. Surprisingly though, Combination 4 (IL-15, anti-CD3e and CXCL10) was nearly as effective a combination, even though CXCL10 was inferior to IL-2 as an individual treatment. Levels of activation markers on the immune cells (FIG. 3C-3D) and IFN-g secretion (FIG. 3E) showed similar results. Combination 1 was selected for further killing assays and therefore the three immunomodulators (IL-2, IL-15 and anti-CD3e) were cloned into output vectors under the control of a minimal promoter linked to Gal4-binding sites (SEQ ID NO: 20-22 with 3 Gal4-BS and SEQ ID NO: 23-25 with 8 Gal4-BS).

### Example 4: Designing Output Construct in Lentivirus

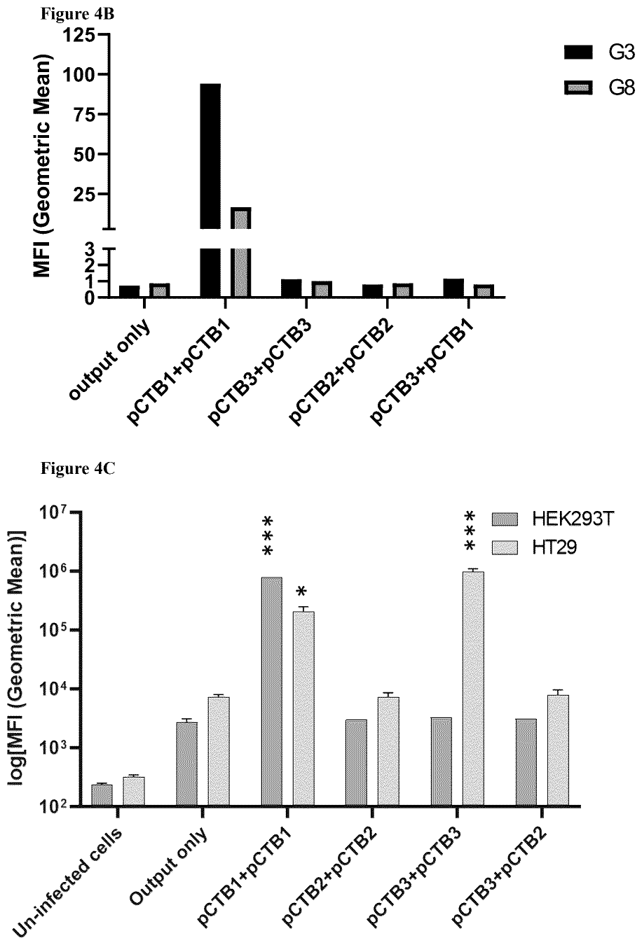

The output construct drives expression of the therapeutic molecule only when the protein complex formed by binding of the two interacting proteins (e.g., Coh2 and DocS) and containing the DNA binding domain (e.g., Gal4 binding domain) and the transcriptional activation domain (e.g., VP16 activation domain) binds to the promoter of the output construct. In this case, the output construct contains repeats of the Gal4 binding site (SEQ ID NO: 17) upstream of the minimal promoter. In order to optimize the output expression two different constructs were tested, one containing three repeats of the binding site (G3; SEQ ID NO: 26) and one containing eight repeats (G8; SEQ ID NO: 27). Various couples of constructs were infected into HT29 CRC cells along with the G3 or the G8 output constructs, and EGFP expression was measured (FIG. 4A). When constitutive promoter constructs were expressed (pCTB1), EGFP expression was equal whether 3 Gal4 binding sites were present or 8. When tissue specific promoters were used (pCTB2) expression was much higher from the G8 construct than the G3. In fact, a combination of a tissue specific promoter and a constitutive promoter also produced increased expression from the G8 construct. When HEK293, non-cancerous cells, were used, no difference was observed in expression between the G3 and G8 constructs (FIG. 4B). A comparison of the expression in the two cell types using the G8 output construct showed the tissue specific nature of the promoters (FIG. 4C). The G8 format for the output construct was thus adopted for all future experiments.

### Example 5: Designing Output Construct in Adenovirus

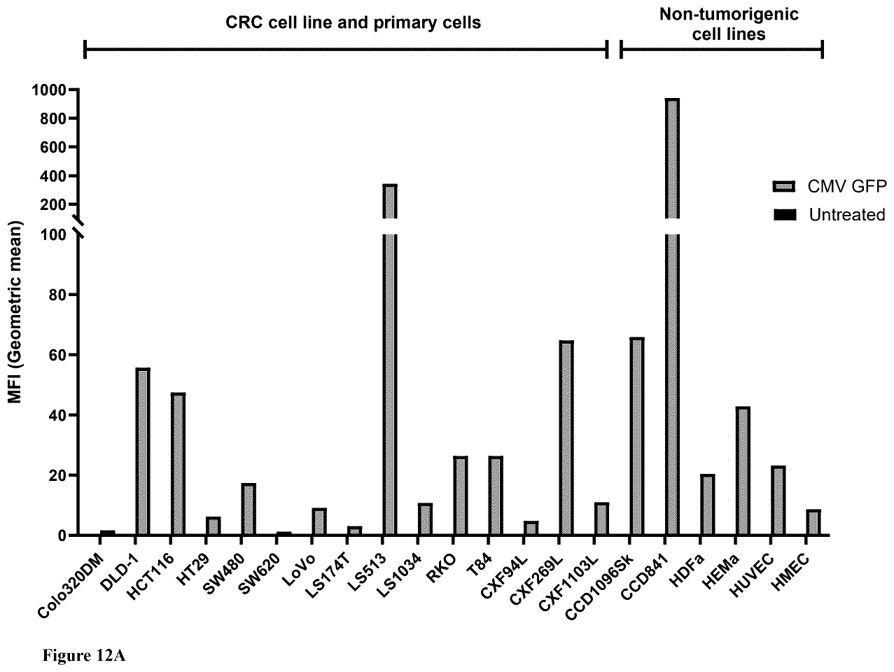

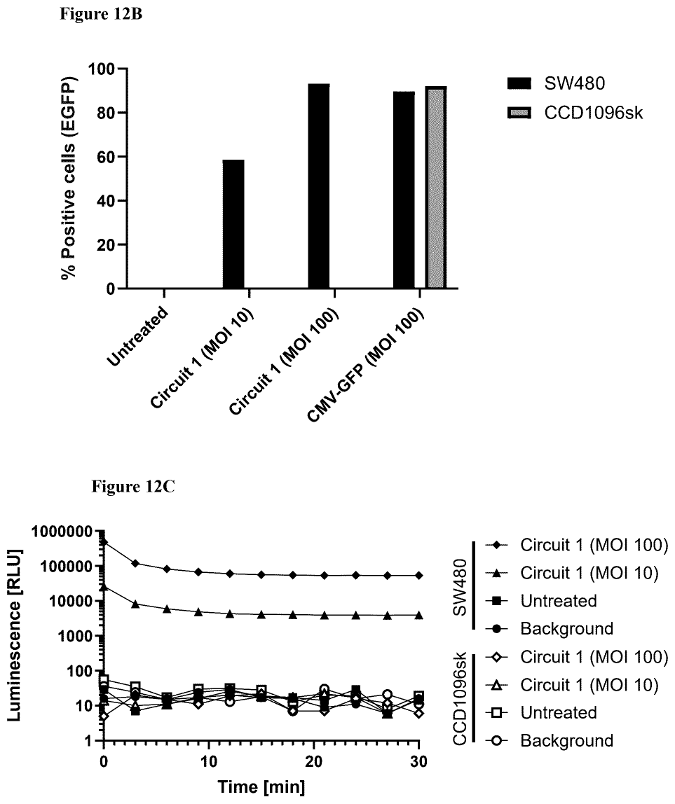

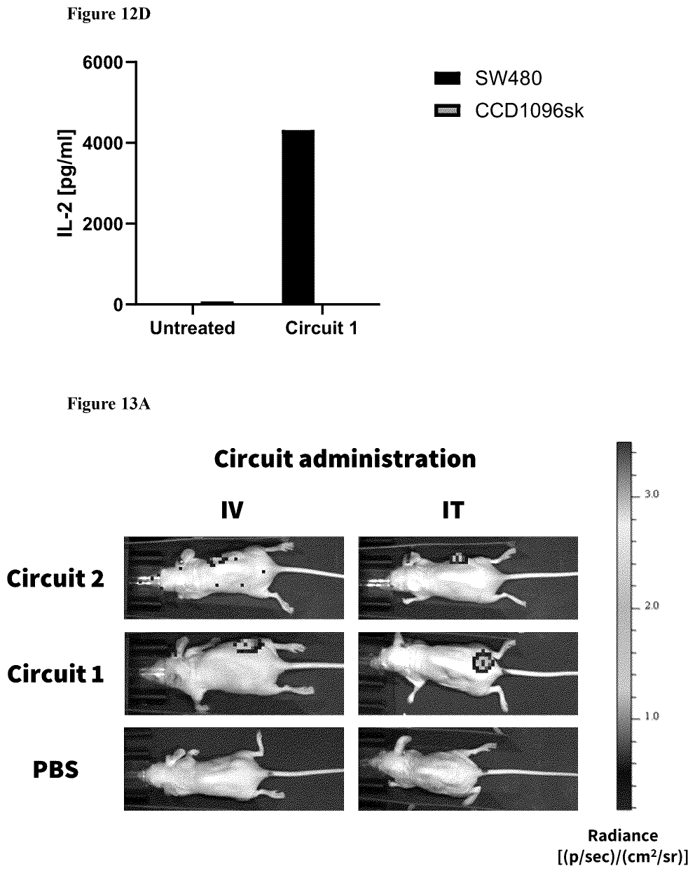

In order to achieve efficient circuit expression from a single virus particle, an expression cassette comprised of either CMV-EGFP or the abovementioned circuit was constructed and inserted into the backbone of adenovirus. Validating the ability to use adenovirus as a vector, the tropism of the CMV-EGFP adenovirus was tested in various CRC cell lines, CRC primary cells and non-cancerous cell lines (FIG. 12A). The adenoviral vector containing CMV-EGFP expressed in all tested cells although with a great deal of variability. To demonstrate the selectivity of the promoters in adenovirus towards CRC cell lines, a circuit using pCTB3 and pCTB7 as inputs (Circuit 1) was tested with three different outputs: EGFP, Firefly-Luciferase and IL-2 and anti-CD3e. These adenoviruses were used to infect either the CRC cell line SW480 or the non-cancerous cell line CCD-1096sk at a MOI (Multiplicity Of Infection) ranging between 10-300. Flow cytometry, luminescence and ELISA were used to measure the outputs of the various circuits used, EGFP, Firefly-Luciferase and IL-2 and anti-CD3e, respectively. The results show that only the SW480 cell line expressed the circuit's output while the circuit produced no appreciable expression in the control cells (FIG. 12B-12D). The CMV-EGFP control was equally expressed in both cell lines (FIG. 12B). Further, the expression from the circuit was dose dependent, as an increase in MOI of an order of magnitude produced an increase in output expression (FIG. 12A-12B). These results demonstrate that the adenovirus can be used similarly to lentivirus.

### Example 6: In Vitro Killing of CRC Cells with the DPI Circuits

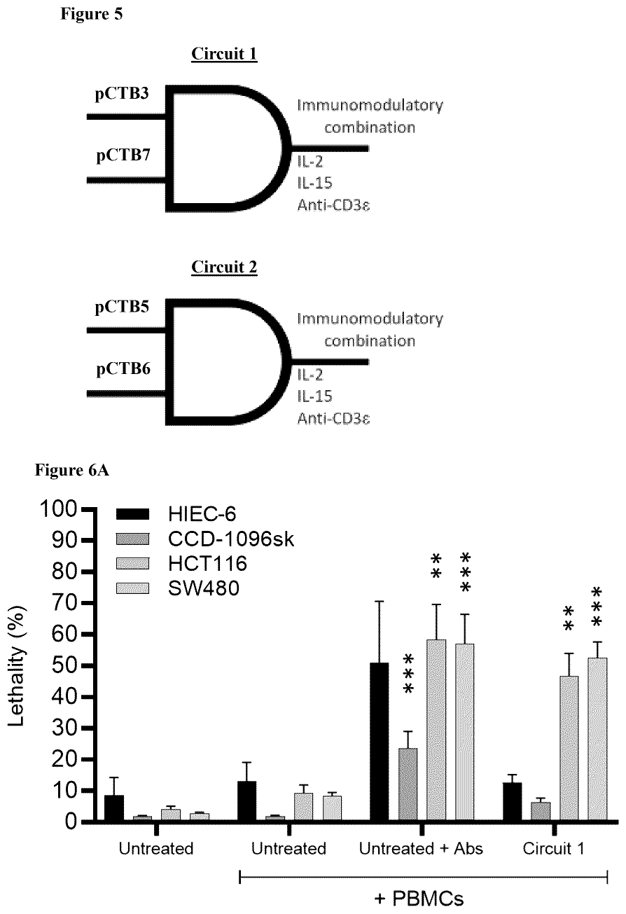

Two circuits for killing CRC cells were built. The first used pCTB3 and pCTB7 as inputs and had three output constructs expressing IL-2, IL-15 and anti-CD3e (Circuit 1). The second used pCTB5 and pCTB6 as inputs and had the same three output constructs. These circuits are depicted visually in FIG. 5.

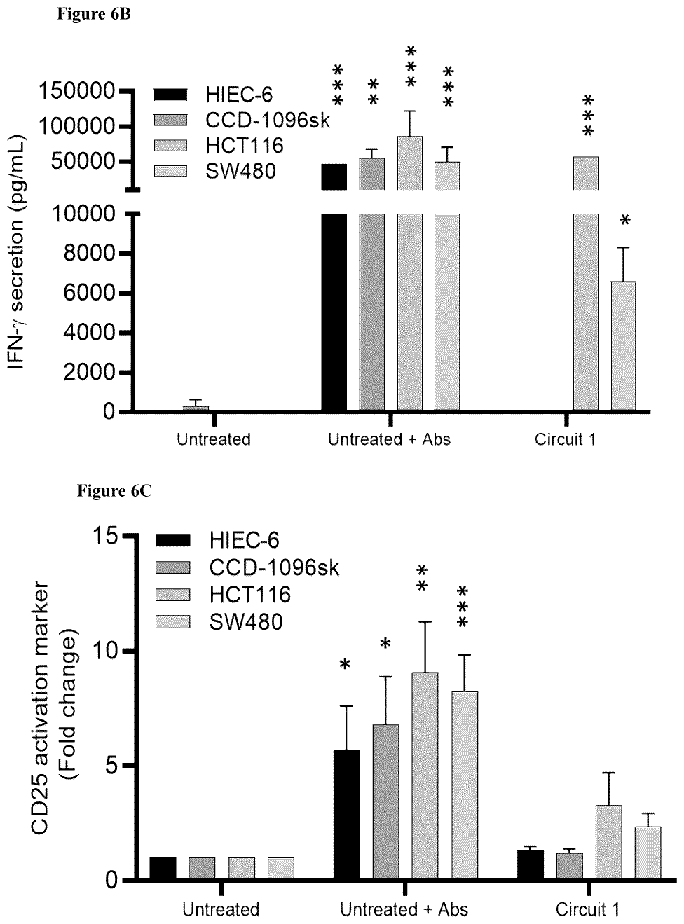

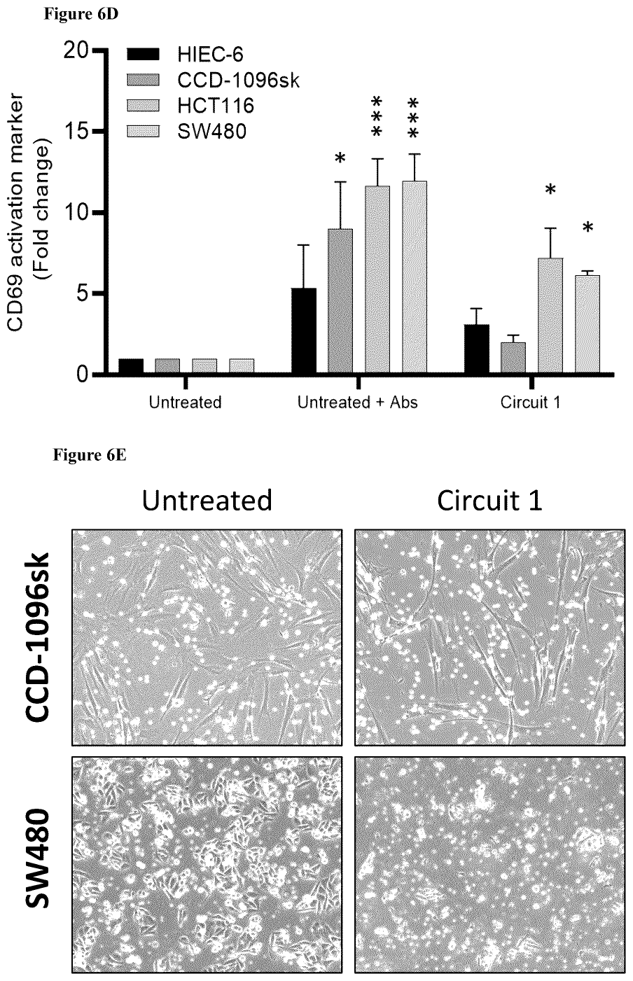

Circuit 1 in lentivirus was used to infect CRC cell lines SW480 and HCT116 as well as the non-tumorigenic colorectal cell line HIEC-6 and the skin cell line CCD-1096sk. The co-culture cytotoxicity cell-based assay was used, as before, to test immune cell killing. The same controls were used as before and lethality, immune activation markers and IFN-g secretion were all measured 72 hours after the start of co-culture. Cell lines cultured alone or with naïve PBMCs showed lethality around of below 10% at 72 hours (FIG. 6A, first two sets of columns) whereas co-culture with PBMCs stimulated with anti-CD3 and anti-CD28 showed robust lethality (greater than 50% for 3 of the lines and greater than 25% for the fourth) of both the CRC cell lines and the non-cancerous cell lines (FIG. 6A, third set of columns). In contrast, CRC cells expressing circuit 1 showed robust lethality comparable to that observed with stimulatory antibodies (greater than 50%), while the non-cancerous cell lines expressing circuit 1 showed lethality comparable to the negative controls (around or below 10%) (FIG. 6A, last set of columns). The same was true for IFNg secretion, which was high in co-culture of all cell lines when the stimulatory antibodies were added but was only observed in co-culture with CRC cells expressing circuit 1 and was completely absent from co-cultures with non-cancerous cells expressing circuit 1 (FIG. 6B). Activation markers CD69 and CD625 showed similar patterns with levels increasing in all incubations with stimulatory antibodies added but only when circuit 1 was expressed in CRC cells and not when expressed in non-cancerous cells (FIG. 6C). The specific cell death in CRC cells is presented visually in FIG. 6D and all of this data taken together demonstrates the CRC specificity of the circuits of the invention and the complete absence of off-target effects in non-cancerous cells.

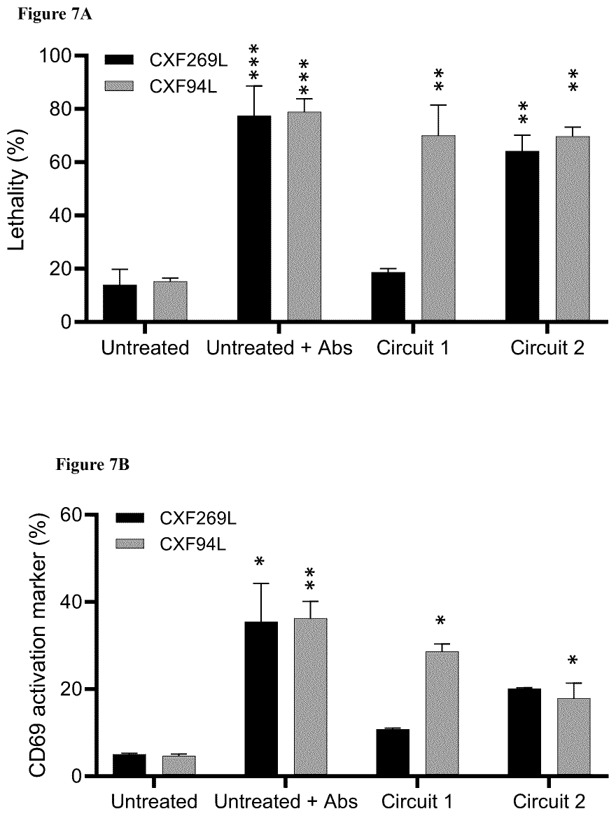

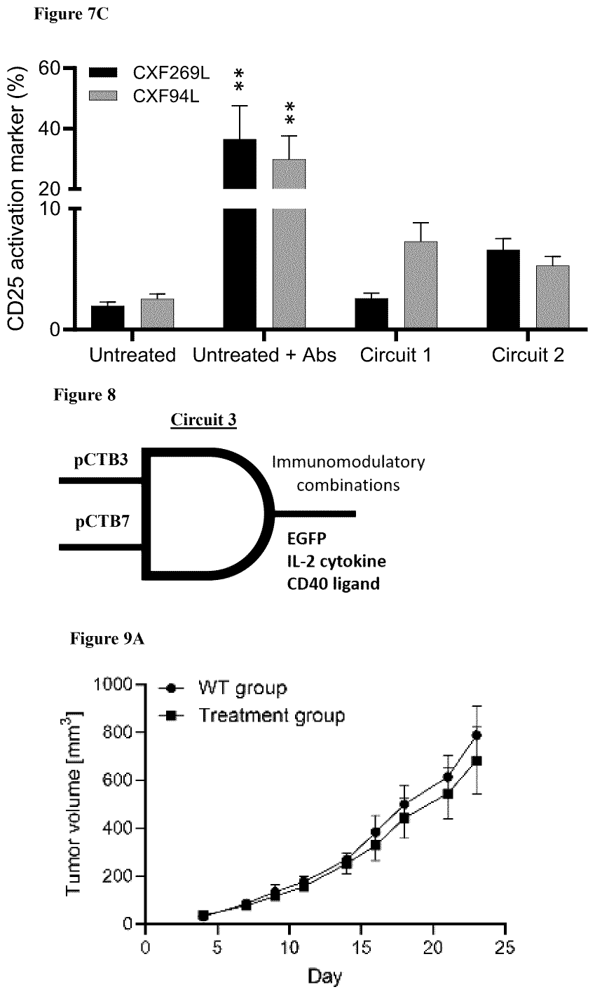

Next, the efficacy of circuit 1 and circuit 2 was tested in patient derived xenograft models (PDX). Two patient derived cell samples CXF94L (Patient sample 1 in FIG. 1) and CXF269L (Patient sample 2 in FIG. 1) were infected with the two lentiviral circuits and co-cultured with naïve PBMCs for 72 hours at an effector to target cell ratio of 10:1. Circuit 2 was highly active in both PDX models and produced increased lethality (FIG. 7A) and immune cell activation (FIG. 7B-7C) that was comparable to treatment with stimulatory antibodies. In contrast, circuit 1 was only active in one of the PDX models (CXF94L/Patient sample 1) and not in the other (FIG. 7A-7C). An examination of FIG. 1 shows why this is so and highlights the combinatorial nature of the circuit. pCTB3 did not express in Patient sample 2 (CXF269L) and so, even though pCTB7 did express in this PDX model, circuit 1 did not produce expression of the immunomodulatory proteins in these cells. Both pCTB5 and pCTB6 expressed in both Patient sample 1 and Patient sample 2 and so circuit 2 was active in both of these PDX models.

Other immunomodulatory output proteins are also examined. Their expression from single promoters and from the DPI circuit is tested. The ability to kill cancer cells both in vitro, in the PDX model and in mice is tested. The promoters and circuits of the invention are able to produce cancer specific expression of the output molecules. The following molecules are tested: IL-15 isoform 2, GM-CSF, FLT3LG, anti-PD1 scFv, CCL21, MICA, ULBP1, 4-1BBL, and OX40L. Various combinations of these output molecules and combinations with IL-2, IL-15 isoform 1, anti-CD3e, CXCL10, CD80, and CD86 are also tested.

### Example 7: In Vivo Monitoring of the DPI Circuits in a Mouse Model

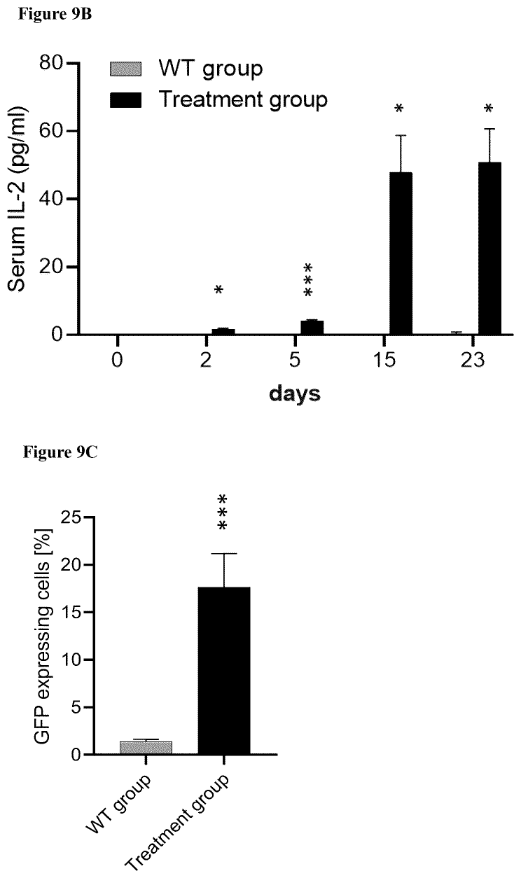

To test the circuit's in vivo activity, NSG mice were chosen for the experiments. NSG mice are a well-documented immunodeficient model for immunotherapy drug development, lacking immune system cells, and so the circuit would not be expected to have a strong effect on the tumor. However, this provides an opportunity to monitor off-target and on-target expression of the immunomodulatory molecules in vivo. First, to test the circuit activity in a mouse model and obtain an engineered cancer cells growth curve, HCT116 colorectal cancer cells were ex vivo infected with Circuit 3 (FIG. 8) in a lentivirus. Circuit 3 makes use of the same two inputs as circuit 1 (pCTB3 and pCTB7), but as output produces EGFP, IL-2 and CD40L (SEQ ID NO: 28-29 for the 3-BS, G3, construct and the 8-BS, G8, constructs respectively). ⅓ of the cells were infected with each output construct. This combination of molecules was selected because EGFP expression allows for real-time monitoring of the cells, IL-2 can be measured in serum using ELISA and CD40L can be detected on the cell surface by Flow cytometry. 1×10\hat{ }6 infected cells (⅓ with the EGFP construct, ⅓ with the IL-2 construct and ⅓ with the CD40L construct) were inoculated into the mouse flank and mice were sacrificed after 23 days. During the experiment period, blood samples were extracted from mice at several time points for future examination of IL-2 serum level detection. At day 23, tumors were excised and measured, dissociated and examined. As can be seen in FIG. 9A, expression of Circuit 3 did not affect tumor size and growth curve compared to untreated HCT116 cells which is to be expected as human immune cells were not administered. As the purpose of this experiment was to monitor output expression, IL-2 secretion by the cancer cells was measured to be sufficiently high that it could already be detected in blood by day 2 and was highly present by day 15 (FIG. 9B). In the excised tumors, ˜20% of cells expressed EGFP in the treatment group (FIG. 9C) indicating the preserved functionality of the circuit overtime. CD40L was undetectable, probably due to the enzymatic activity of the tumor dissociation kit, removing cell surface proteins. EGFP and CD40L are not detected in any non-cancerous cells.

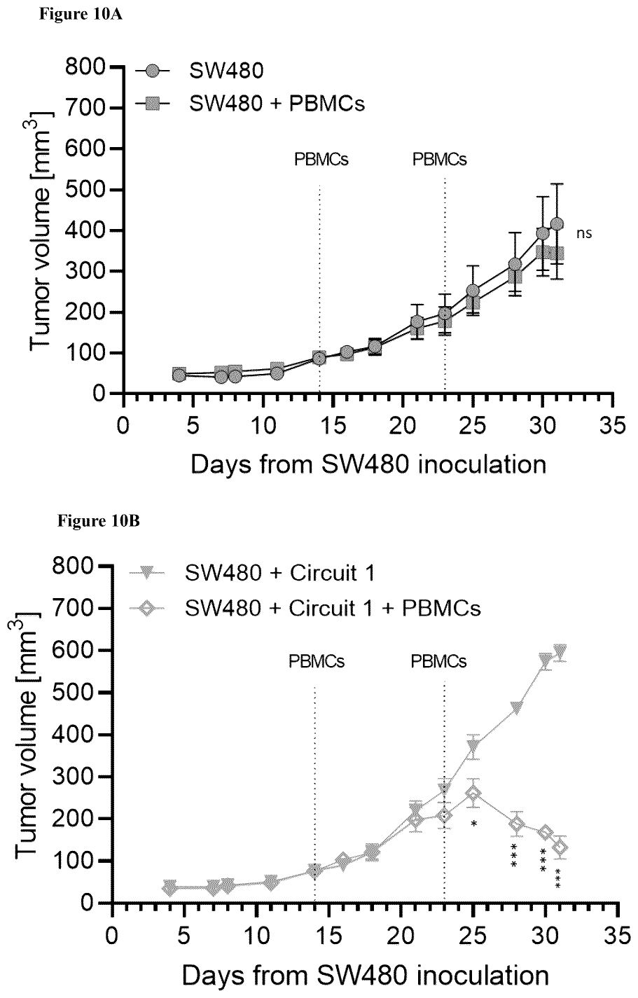

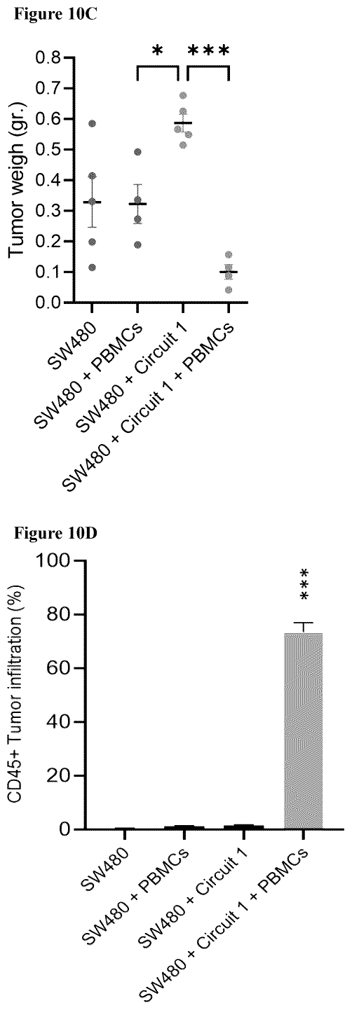

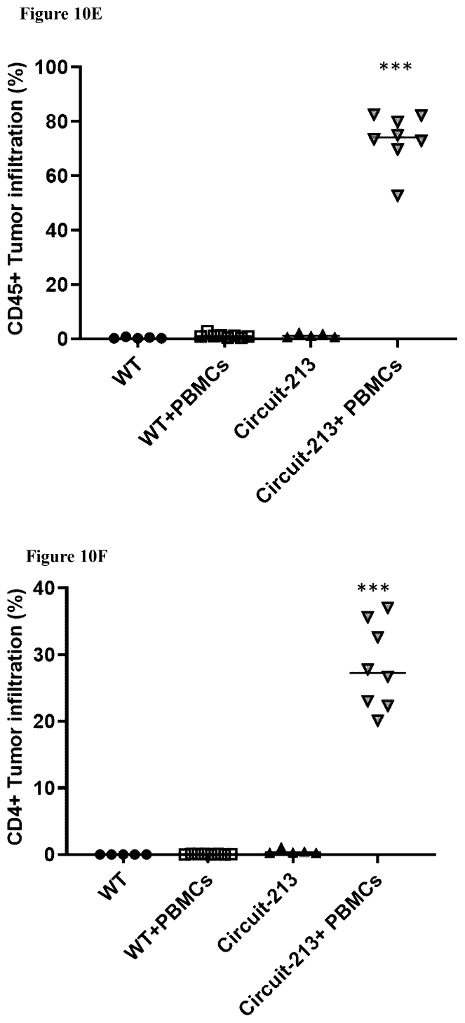

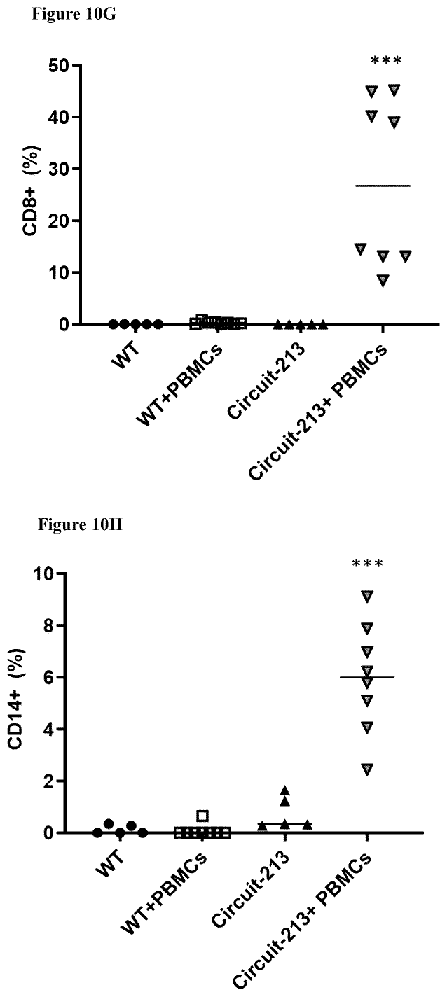

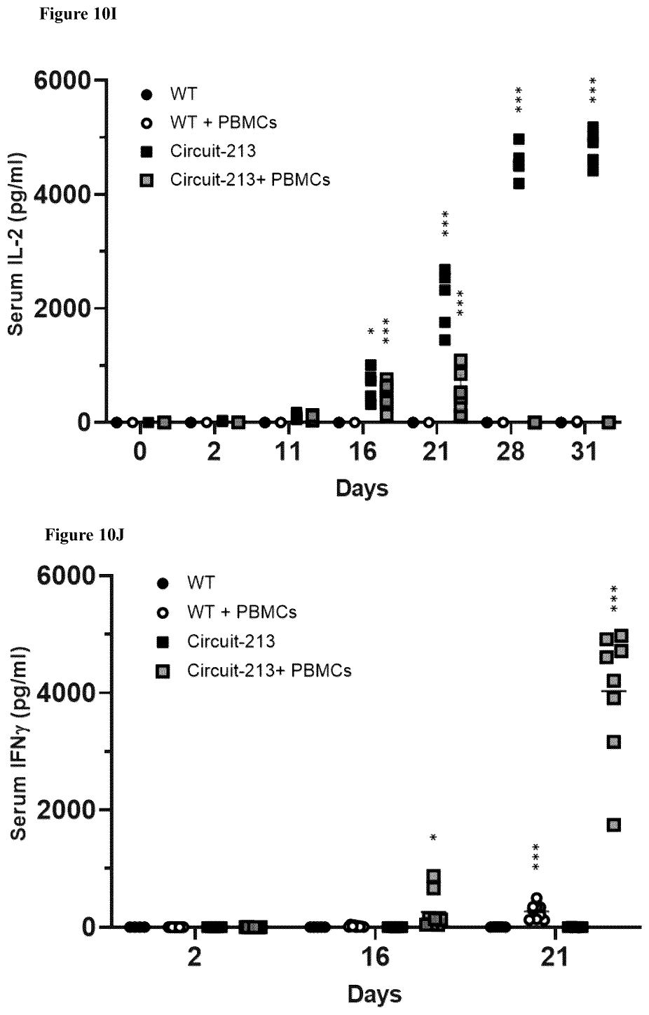

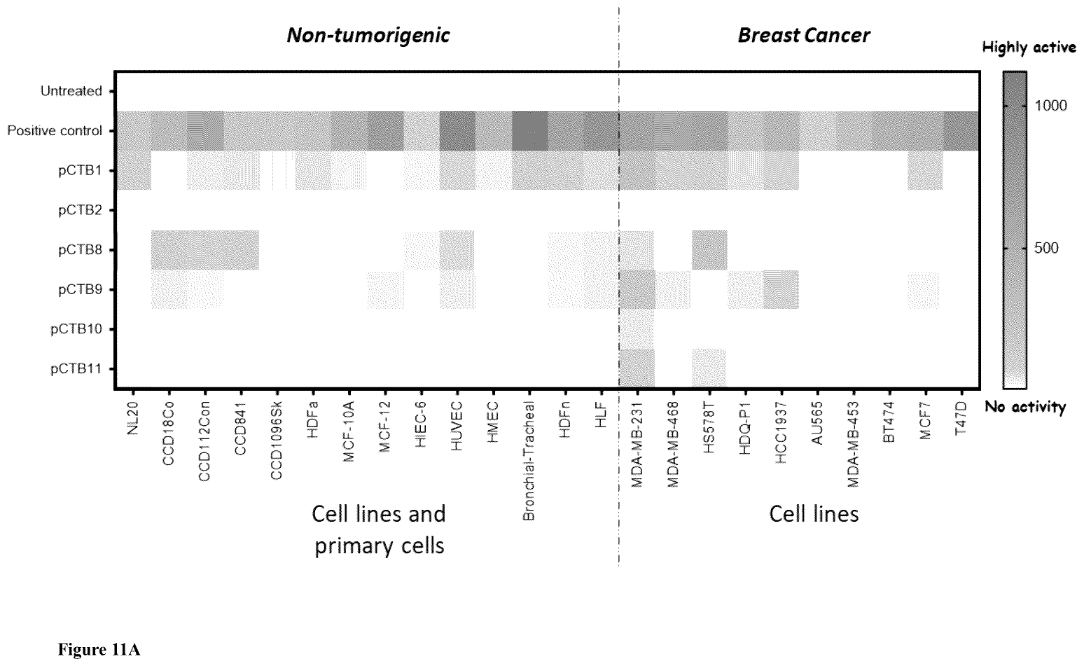

In a follow up experiment, SW480 cells either infected with Circuit 1 or untreated were injected into the flank of NSG mice. Two weeks later naïve human PBMCs were administered. A second administration of PBMC also occurred at day 23. Tumor size was followed until day 31 at which point tumors were weighed and the infiltration of CD45 positive immune cells was assessed. Untreated SW480 cells produced a steadily growing tumor that was barely affected by the administration of PBMCs (FIG. 10A). In contrast, the CRC cells expressing Circuit 1 produced a steadily growing tumor in the mouse, but at around day 23, when the second administration of human PBMCs took place, the beginning of a sharp and robust shrinking of the tumor took place which proceeded until the mice were sacrificed at day 31 (FIG. 10B). This was reflected in the final tumor weights, where a major reduction in weight (78%) was observed only with expression of Circuit 1 and administration of human PBMCs (FIG. 10C). When the tumors were examined by flow cytometry for CD45 positive cells, these cells were found to be absent when untreated CRC cells were administered, or when CRC cells expressing Circuit 1 were administered without human PBMCs. However, robust immune cell infiltration was observed when CRC cells expressing Circuit 1 were administered with human PBMCs (FIG. 10D). Indeed, 85% of cells in the tumor were CD45 positive, indicating that much of the tumor volume was actually immune cells. Immunophenotyping cell surface markers on PBMCs from the residual tumors and TME was performed via flow cytometry (FIG. 10E-10H). All examined types of immune cells (CD4+, CD8+, CD14+) were found to infiltrate the tumor when CRC cells expressing Circuit 1 were administered with PBMCs. IL-2 expression from cancer cells was measured from blood serum, showing some correlation with tumor size (FIG. 10I). INFg expression by activated PBMCs was measured from blood serum, showing activation starting from day 16 (FIG. 10J). This indicates that the circuit was effective in modulating the TME and producing an immunogenic environment and thereby shrinking the tumor.

### Example 8: In-Vivo Bio-Distribution

In-vivo bio-distribution was now studied using the adenoviral constructs. The primary objective was to track the systemic expression of the circuits in an animal model over time, thereby evaluating their selectivity towards CRC. Therefore, the output of Circuit 1 and Circuit 2 was Firefly-Luciferase, commonly used to monitor the output signal across various organs and tissues via a non-invasive bio-luminescence imaging technique. Balb/c Nude mice were chosen as the model for the experiments.

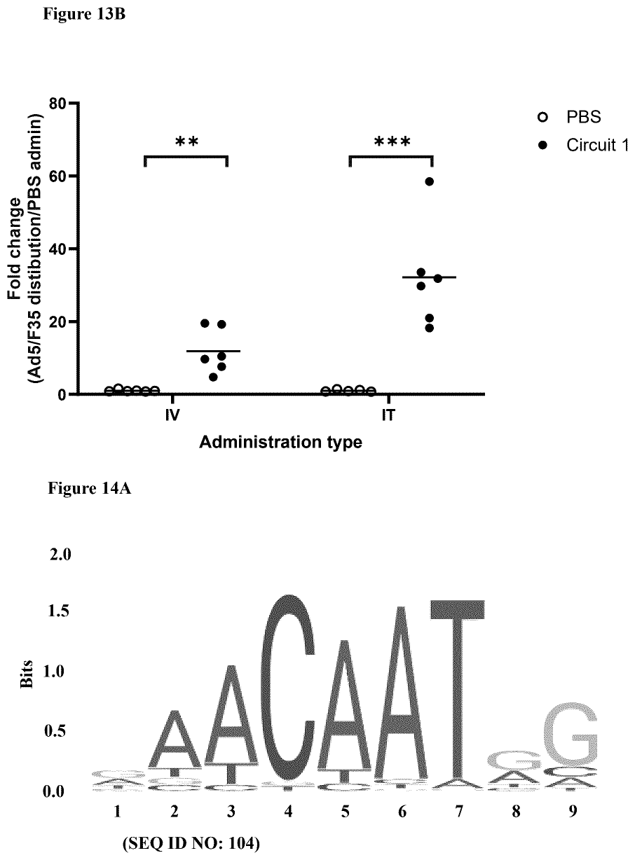

SW480 cells were injected into the flank of the mice. 14 days later, mice were injected either with PBS or with viruses harboring Circuit 1 or Circuit 2. Injection was either intravenously (IV) using titers of 5*10\hat{ }8 or 3*10\hat{ }9 or intratumorally (IT) using titers of 1*10\hat{ }9 or 1*10\hat{ }8 for Circuit 1 and Circuit 2, respectively. The results after 5 days (for Circuit 1) or 9 days (for Circuit 2) depict luciferase expression exclusively within the tumors (FIG. 13A). At the end of the experiment (day 12), adenovirus DNA was found in tumor cells from mice injected with adenovirus regardless of route of administration, i.e. in both IT and repeated IV (FIG. 13B). These results taken together confirm the circuit's capacity to penetrate the tumor microenvironment and reach the cancer cells, and the minimization of off-target effects in healthy tissues.

### Example 9: Breast Cancer Specific Promoters and Circuits

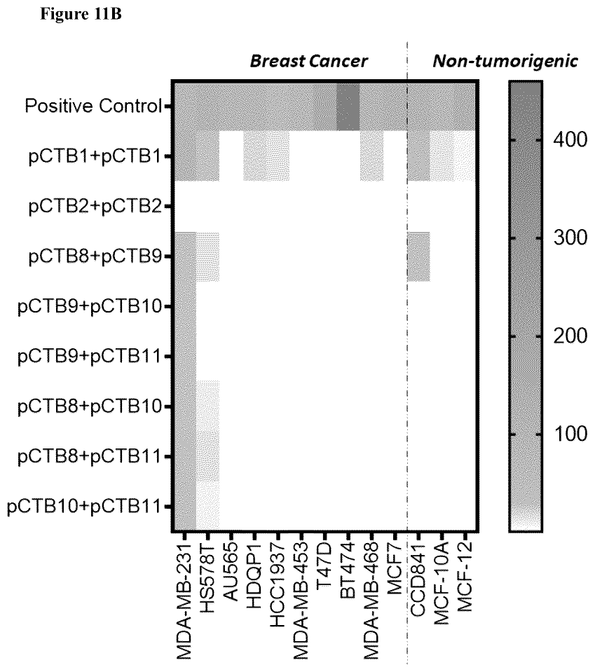

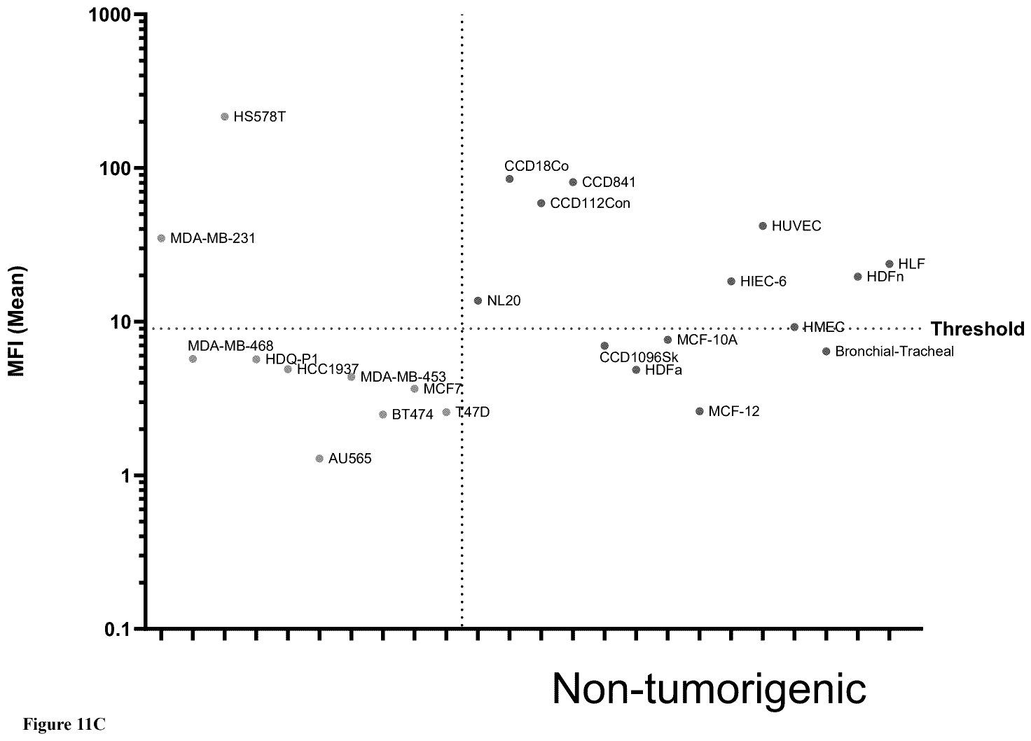

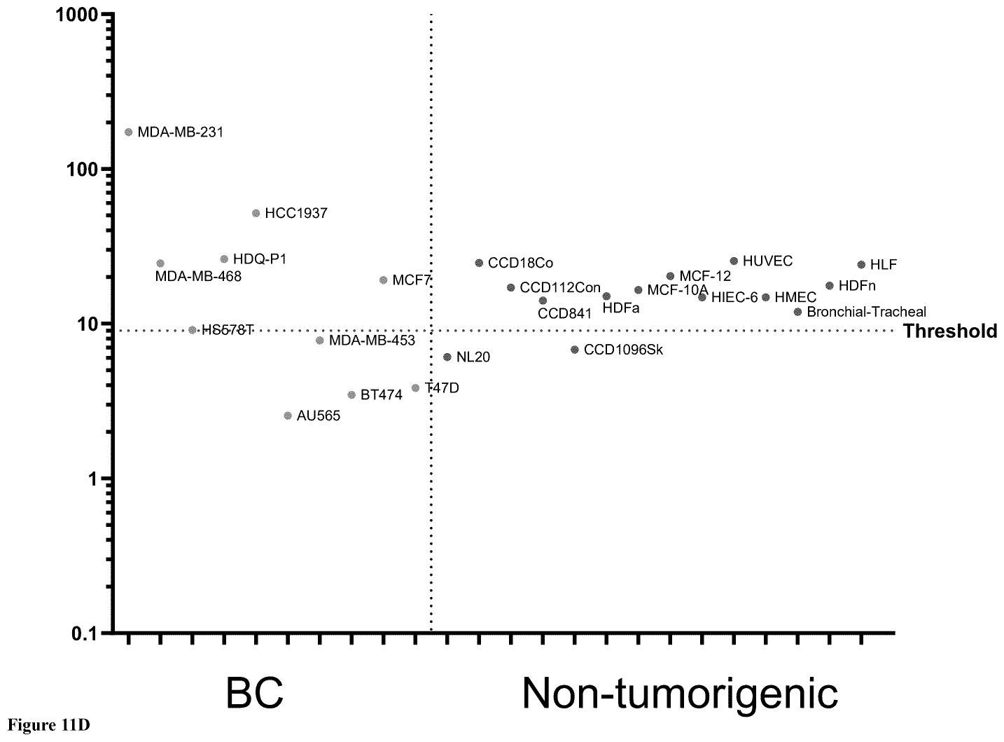

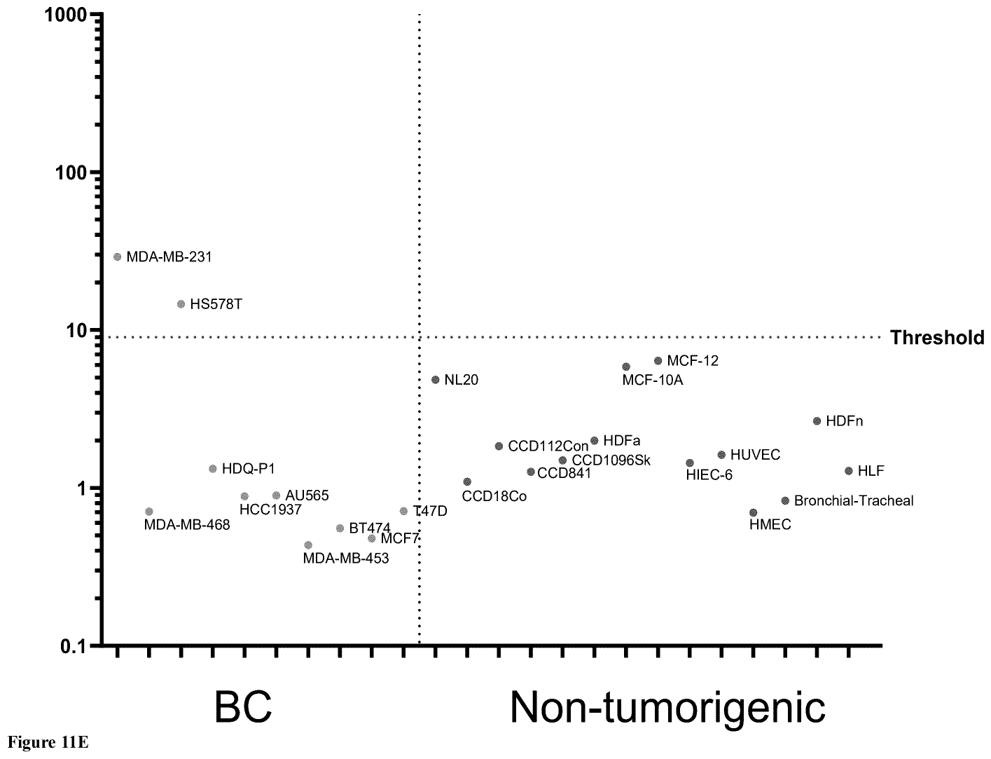

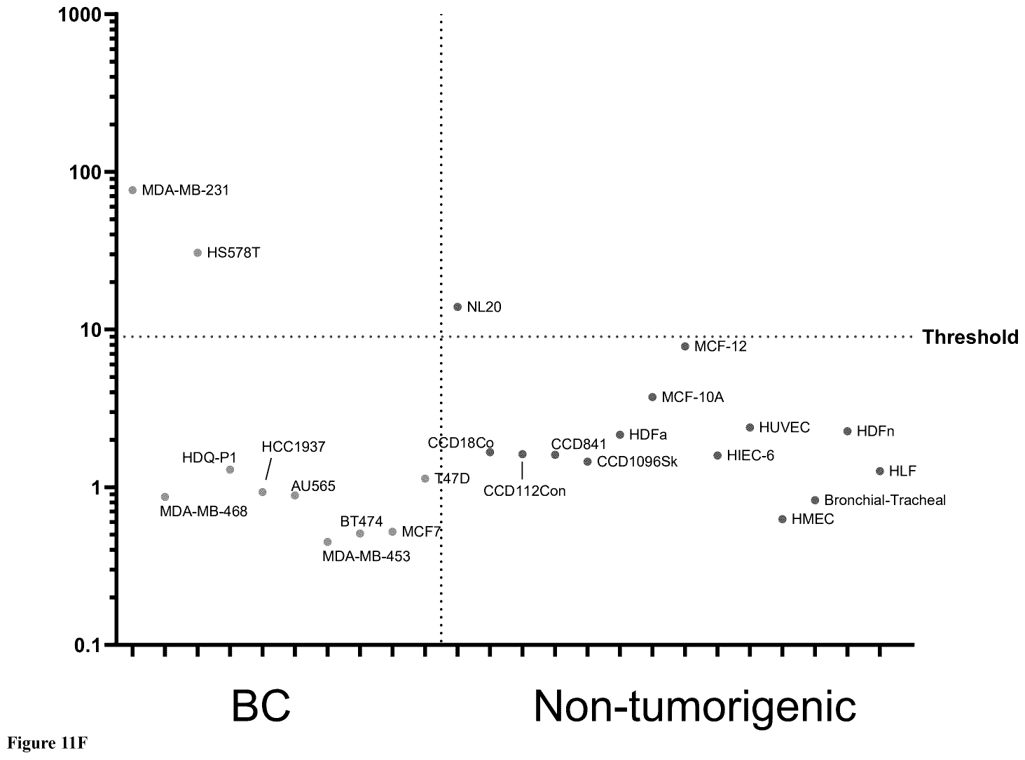

The same workflow used to design the CRC-specific DPI system was used to select breast cancer specific promoters and a breast cancer specific DPI system. Four transcription factor binding site repeat regions were selected from the initial screen as potentially having breast cancer specific expression. These 4, pCTB8-11, were tested in 10 breast cancer cells lines as well as all aforementioned non-tumorigenic cell lines (FIG. 11A). Two of them, pCTB8 and pCTB9, though expressing strongly in many breast cancer cell lines also expressed in non-tumorigenic cells and thus were not considered breast cancer specific. The other two, pCTB10 and pCTB11, did not express or very lowly expressed in the non-tumorigenic cells and did show expression, though restricted, in some breast cancer cell lines. A cutoff for positive expression (MFI 9) was determined (FIG. 11C-11F) and was found to be useful for all 4 promoters. Promoters pCTB10 and pCTB11 showed positive expression in almost no healthy cells and did show expression in some breast cancer cell lines. Therefore, it was determined that these synthetic promoters are indeed BC-specific. The other two promoters, while showing strong expression in many breast cancer cell lines, could not be considered BC-specific due to high expression in a number of non-tumorigenic cell lines. The individual TF-BSs present in the two BC-specific syntenic promoters as well as the full sequences of the TF-BS regions are summarized in Table 3.

TABLE 3
synthetic promoter sequences, BC-specific

| Synthetic | | | |
|---|---|---|---|
| promoter | TF | BS (SEQ ID NO:) | Promoter region |
| pCTB10 | MYBL2 | AACGGTTAACGGTT (6) | SEQ ID NO: 13 |
| pCTB11 | MYB | TTGACCGTTA (7) | SEQ ID NO: 14 |

DPIs were constructed with various combinations of the breast cancer promoters and expression in breast cancer cells and non-tumorigenic cells was measured. FIG. 11B emphasizes the effectiveness of the DPI system as, any system containing pCTB10 or pCBT11 showed very low or no expression in non-tumorigenic cells even when the second promoter produced off-target expression. Regardless, the combination of the two breast cancer specific input constructs, pCTB10 and pCTB11, was determined to have the best specificity and expression in breast cancer cells. The promoters are also tested in 3 patient derived breast cancer cell lines (br-98, br-99 and b2-103) and are found to be active in these cells as well.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A nucleic acid molecule configured to specifically express a sequence of interest in a cancer cell, the molecule comprising: a transcription factor binding region comprising 3-10 repeats of a transcription factor binding site, a minimal promoter, and said sequence of interest operatively linked to said transcription factor binding region and minimal promoter, wherein said transcription factor is selected from SOX9, GATA10, TCF7L1, LEF1, MYBL2 and MYB.

2. The nucleic acid molecule of claim 1, wherein
a. said transcription factor is SOX9 and said transcription factor binding site comprises NAACAAWRG (SEQ ID NO: 104) or its reverse complement;
b. said transcription factor is GATA10 and said transcription factor binding site comprises YBHCYBHDWTHW (SEQ ID NO: 106) or its reverse complement;
c. said transcription factor is TCF7L1 and said transcription factor binding site comprises YCTTTGATSTY (SEQ ID NO: 108) or its reverse complement;
d. said transcription factor is LEF1 and said transcription factor binding site comprises NNASATCAAAGNNNN (SEQ ID NO: 110) or its reverse complement or CCTTTGAT (SEQ ID NO: 112) or its reverse complement;
e. said transcription factor is MYBL2 and said transcription factor binding site comprises RACSGTTAACSKHY (SEQ ID NO: 113) or its reverse complement; or
f. said transcription factor is MYB and said transcription factor binding site comprises NNDDCCGTTD (SEQ ID NO: 115) or its reverse complement.

3. The nucleic acid molecule of claim 2, wherein
a. said transcription factor is SOX9 and said transcription factor binding site comprises or consists of GAACAAAGGCCCCTTT (SEQ ID NO: 1) or its reverse complement;
b. said transcription factor is GATA10 and said transcription factor binding site comprises or consists of TCCCTTTTATCT (SEQ ID NO: 2) or its reverse complement;
c. said transcription factor is TCF7L1 and said transcription factor binding site comprises or consist of TGCCTTTGATGTT (SEQ ID NO: 3) or its reverse complement;
d. said transcription factor is LEF1 and said transcription factor binding site comprises or consists of AAAGATCAAAGGGTT (SEQ ID NO: 4) or its reverse complement or TCCTTTGATTTGCT (SEQ ID NO: 5) or its reverse complement;
e. said transcription factor is MYBL2 and said transcription factor binding site comprises or consists of AACGGTTAACGGTT (SEQ ID NO: 6) or its reverse complement; or
f. said transcription factor is MYB and said transcription factor binding site comprises or consists of TTGACCGTTA (SEQ ID NO: 7) or its reverse complement.

4. The nucleic acid molecule of claim 1, wherein said 3-10 repeats are all binding sites of the same transcription factor, are all identical or both.

5. The nucleic acid molecule of claim 1, wherein said 3-10 repeats are 6-9 repeats.

6. The nucleic acid molecule of claim 1, wherein at least one of:
a. said transcription factor binding region comprises a spacer of between 1-5 nucleotides between each repeat;
b. said transcription factor binding region comprises a spacer consisting of 3 nucleotides between each repeat;
c. said transcription factor binding region consists of between 100 and 150 nucleotides;
d. said transcription factor binding region consists of between 110 and 125 nucleotides;
e. said minimal promoter is selected from the late adenoviral promoter and the YB_TATA promoter;
f. said specific expression comprises off target expression in non-cancerous cells below a predetermined threshold;
g. the nucleic acid molecule further comprises a 5′ untranslated region (UTR) between said minimal promoter and said sequence of interest;
h. said sequence of interest encodes a therapeutic molecule configured to treat said cancer; and
i. said nucleic acid molecule is an expression vector.

7. The nucleic acid molecule of claim 1, wherein said transcription factor is selected from SOX9, GATA10, TCF7L1, and LEF1 and said nucleic acid molecule is configured to specifically express said sequence of interest in a colorectal cancer cell or said transcription factor is selected from SOX9, MYBL2 and MYB and said nucleic acid molecule is configured to specifically express said sequence of interest in a breast cancer cell.

8. The nucleic acid molecule of claim 1, wherein said transcription factor binding region comprises any one of SEQ ID NO: 8-14.

9. A nucleic acid molecule system comprising:
a. a first nucleic acid molecule of claim 1, wherein said sequence of interest encodes a first fusion protein comprising a DNA binding domain and a first protein binding domain;
b. a second nucleic acid molecule of claim 1, wherein said sequence of interest encodes a second fusion protein comprising a transcriptional activation domain and a second protein binding domain; and
c. a third nucleic acid molecule comprising a polynucleotide operably linked to a transcriptional regulatory element, wherein said DNA binding domain binds to said transcriptional regulatory element;
wherein said first protein binding domain and said second protein binding domain are configured to bind to each other, and wherein said first nucleic acid molecule and said second nucleic acid molecule comprise repeats of different transcription factor binding sites.

10. The nucleic acid molecule system of claim 9, wherein said polynucleotide comprises an open reading frame encoding a protein or wherein said polynucleotide comprises a sequence of a non-coding RNA.

11. The nucleic acid molecule system of claim 10, wherein
a. said protein is a reporter protein;
b. said protein is a therapeutic protein;
c. said non-coding RNA is a therapeutic non-coding RNA;
d. said protein is an anticancer protein, toxin or proapoptotic peptide.

12. The nucleic acid molecule system of claim 10, wherein said protein is an immunomodulatory protein selected from interleukin-15 (IL-15), IL-2, an anti-CD3e binding agent and a combination thereof.

13. The nucleic acid molecule system of claim 9, wherein said transcriptional regulatory element is a promoter and recruitment of said transcriptional activation domain to said transcriptional regulatory element drives transcription of said polynucleotide.

14. The nucleic acid construct system of claim 9, wherein at least one of:
a. said first and second protein binding domains are a DocS protein domain and a Coh2 protein domain or are a HrpS protein and a HrpR protein;
b. said first and second protein binding domains are a HrpS protein and a HrpR protein, and wherein said system further comprises a fourth nucleic acid molecule comprising a coding region encoding an HrpV protein operatively linked to a promoter configured to specifically express in non-cancerous cells;
c. said DNA binding domain is a Gal4-DNA binding domain and wherein said transcriptional regulatory element comprises a Gal4 binding site; and
d. said transcriptional activation domain is the VP16 activation domain (VP16-AD) or VP64 activation domain (VP64-AD).

15. The nucleic acid molecule system of claim 9, wherein said first nucleic acid molecule comprises repeats of a LEF1 binding site and said second nucleic acid molecule comprises repeats of a SOX9 binding site or repeats of a TCF7L1 binding site.

16. The nucleic acid molecule system of claim 15, wherein
a. said first nucleic acid molecule comprises repeats of SEQ ID NO: 5 and said second nucleic acid molecule comprises repeats of SEQ ID NO: 1;
b. said first nucleic acid molecule comprises SEQ ID NO: 12 and said second nucleic acid molecule comprises SEQ ID NO: 8;
c. said first nucleic acid molecule comprises repeats of SEQ ID NO: 4 and said second nucleic acid molecule comprises repeats of SEQ ID NO: 3; or
d. said first nucleic acid molecule comprises SEQ ID NO: 11 and said second nucleic acid molecule comprises SEQ ID NO: 10.

17. The nucleic acid molecule system of claim 9, wherein said first nucleic acid molecule comprises repeats of a MYBL2 binding site and said second nucleic acid molecule comprises repeats of a MYB binding site.

18. The nucleic acid molecule system of claim 17, wherein said first nucleic acid molecule comprises repeats of SEQ ID NO: 6 and said second nucleic acid molecule comprises repeats of SEQ ID NO: 7, or said first nucleic acid molecule comprises SEQ ID NO: 13 and said second nucleic acid molecule comprises SEQ ID NO: 14.

19. A pharmaceutical composition comprising a nucleic acid molecule of claim 1 being an expression vector, and a pharmaceutically acceptable carrier, excipient or adjuvant.

20. A method of treating cancer in a subject in need thereof, the method comprising administering to said subject a pharmaceutical composition of claim 19, wherein said nucleic acid molecule comprises binding sites for at least one of SOX9, GATA10, TCF7L1, and LEF1 and said cancer is colorectal cancer or said nucleic acid molecule comprises binding sites for at least one of MYBL2 and MYB and said cancer is breast cancer, thereby treating cancer.


SEQUENCE LISTING (115 sequences)

SEQ ID NO: 1 - DNA, 16 bp, synthetic construct
  source 1..16: mol_type = other DNA
gaacaaaggccccttt

SEQ ID NO: 2 - DNA, 12 bp, synthetic construct
  source 1..12: mol_type = other DNA
tcccttttatct

SEQ ID NO: 3 - DNA, 13 bp, synthetic construct
  source 1..13: mol_type = other DNA
tgcctttgatgtt

SEQ ID NO: 4 - DNA, 15 bp, synthetic construct
  source 1..15: mol_type = other DNA
aaagatcaaagggtt

SEQ ID NO: 5 - DNA, 14 bp, synthetic construct
  source 1..14: mol_type = other DNA
tcctttgatttgct

SEQ ID NO: 6 - DNA, 14 bp, synthetic construct
  source 1..14: mol_type = other DNA
aacggttaacggtt

SEQ ID NO: 7 - DNA, 10 bp, synthetic construct
  source 1..10: mol_type = other DNA
ttgaccgtta

SEQ ID NO: 8 - DNA, 111 bp, synthetic construct
  source 1..111: mol_type = other DNA
gaacaaaggcccctttatcgaacaaaggcccctttgctgaacaaaggccccttttgagaacaaaggcccctttcaggaacaaaggcccctttatggaacaaaggccccttt

SEQ ID NO: 9 - DNA, 117 bp, synthetic construct
  source 1..117: mol_type = other DNA
tcccttttatctatctcccttttatctgcttcccttttatcttgatcccttttatctcagtcccttttatctatgtcccttttatctcgttcccttttatcttactcccttttatct

SEQ ID NO: 10 - DNA, 125 bp, synthetic construct
  source 1..125: mol_type = other DNA
tgcctttgatgttatctgcctttgatgttgcttgcctttgatgtttgatgcctttgatgttcagtgcctttgatgttatgtgcctttgatgttcgttgcctttgatgtttactgcctttgatgtt

SEQ ID NO: 11 - DNA, 123 bp, synthetic construct
  source 1..123: mol_type = other DNA
aaagatcaaagggttatcaaagatcaaagggttgctaaagatcaaagggtttgaaaagatcaaagggttcagaaagatcaaagggttatgaaagatcaaagggttcgtaaagatcaaagggtt

SEQ ID NO: 12 - DNA, 116 bp, synthetic construct
  source 1..116: mol_type = other DNA
tcctttgatttgctatctcctttgatttgctgcttcctttgatttgcttgatcctttgatttgctcagtcctttgatttgctatgtcctttgatttgctcgttcctttgatttgct

SEQ ID NO: 13 - DNA, 116 bp, synthetic construct
  source 1..116: mol_type = other DNA
aacggttaacggttatcaacggttaacggttgctaacggttaacggtttgaaacggttaacggttcagaacggttaacggttatgaacggttaacggttcgtaacggttaacggtt

SEQ ID NO: 14 - DNA, 114 bp, synthetic construct
  source 1..114: mol_type = other DNA
ttgaccgttaatcttgaccgttagctttgaccgttatgattgaccgttacagttgaccgttaatgttgaccgttacgtttgaccgttatacttgaccgttagaattgaccgtta

SEQ ID NO: 15 - protein, 121 aa, synthetic construct
MSTAPPTDVSLGDELHLDGEDVAMAHADALDDFDLDMLGDGDSPGPGWPAGTSTKLYGDVNDDGKVNSTDAVALKRYVLRSGISINTDNADLNEDGRVNSTDLGILKRYILKEIDTLPYKN

SEQ ID NO: 16 - protein, 287 aa, synthetic construct
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSHMVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNV

SEQ ID NO: 17 - DNA, 17 bp, synthetic construct
  source 1..17: mol_type = other DNA
cggagtactgtcctccg

SEQ ID NO: 18 - DNA, 41 bp, synthetic construct
  source 1..41: mol_type = other DNA
ggggggctataaaagggggtgggggcgttcgtcctcactct

SEQ ID NO: 19 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 20 - DNA, 622 bp, synthetic construct
  source 1..622: mol_type = other DNA
cggagtactgtcctccgagcggagtactgtcctccgactcgagcggagtactgtcctccgaagacgctagcggggggctataaaagggggtgggggcgttcgtcctcactctagatctgcgatctaagtaagcttggcattccggtactgtaccggtgccaccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 21 - DNA, 649 bp, synthetic construct
  source 1..649: mol_type = other DNA
cggagtactgtcctccgagcggagtactgtcctccgactcgagcggagtactgtcctccgaagacgctagcggggggctataaaagggggtgggggcgttcgtcctcactctagatctgcgatctaagtaagcttggcattccggtactgtaccggtgccaccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 22 - DNA, 2038 bp, synthetic construct
  source 1..2038: mol_type = other DNA
cggagtactgtcctccgagcggagtactgtcctccgactcgagcggagtactgtcctccgaagacgctagcggggggctataaaagggggtgggggcgttcgtcctcactctagatctgcgatctaagtaagcttggcattccggtactgtaccggtgccaccatgcggctctgcatcccgcaggtgctgttggccttgttcctttccatgctgacagggccgggagaaggcagctacccttacgatgtacctgattacgccggcggcggaggctcacaagtgcagcttcagcagagcggcgctgagctggcccggcccggggcctctgtgaaaatgagctgcaaggcttccggttacacctttacaaggtacacaatgcactgggtgaagcagcggccagggcaggggctcgagtggatcggctacataaacccatctcggggatatacaaactacaatcaaaagttcaaagacaaggccacactgacaacggacaagtcaagcagcacagcttacatgcaactgagctctctgacatccgaggatagcgccgtttactattgcgccagatactatgatgaccactattgcttggactactggggacagggaacaaccctcacagttagctccggcggggggggcagtggaggtggaggatctgggggcggcggtagtcagatcgtcctcacacagtctccggccataatgtccgcctcccccggagagaaggttactatgacatgttccgcatcttcctctgtgtcatatatgaattggtaccagcagaagagtggcacctctcctaaacgctggatttacgatacctctaaactggcgtccggggtgcctgcacatttccgcggatccggctccggtacgagttattcactcacaatatctggaatggaggccgaagatgccgctacttactactgccaacaatggtcaagcaaccccttcactttcgggagcgggacaaagctggagatcaaccaccatcatcatcaccatcatgcaggtatggcctcctctgggtatgtcctccaggcggagctctccccctcaactgagaactcaagtcagctggacttcgaagatgtatggaattcttcctatggtgtgaatgattccttcccagatggagactatgatgccaacctggaagcagctgccccctgccactcctgtaacctgctggatgactctgcactgcccttcttcatcctcaccagtgtcctgggtatcctagctagcagcactgtcctcttcatgcttttcagacctctcttccgctggcagctctgccctggctggcctgtcctggcacagctggctgtgggcagtgccctcttcagcattgtggtgcccgtcttggccccagggctaggtagcactcgcagctctgccctgtgtagcctgggctactgtgtctggtatggctcagcctttgcccaggctttgctgctagggtgccatgcctccctgggccacagactgggtgcaggccaggtcccaggcctcaccctggggctcactgtgggaatttggggagtggctgccctactgacactgcctgtcaccctggccagtggtgcttctggtggactctgcaccctgatatacagcacggagctgaaggctttgcaggccacacacactgtagcctgtcttgccatctttgtcttgttgccattgggtttgtttggagccaaggggctgaagaaggcattgggtatggggccaggcccctggatgaatatcctgtgggcctggtttattttctggtggcctcatggggtggttctaggactggatttcctggtgaggtccaagctgttgctgttgtcaacatgtctggcccagcaggctctggacctgctgctgaacctggcagaagccctggcaattttgcactgtgtggctacgcccctgctcctcgccctattctgccaccaggccacccgcaccctcttgccctctctgcccctccctgaaggatggtcttctcatctggacacccttggaagcaaatcc

SEQ ID NO: 23 - DNA, 731 bp, synthetic construct
  source 1..731: mol_type = other DNA
cggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccggctagcggggggctataaaagggggtgggggcgttcgtcctcactctagatctgcgatctaagtaagcttggcattccggtactgtaccggtgccaccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 24 - DNA, 758 bp, synthetic construct
  source 1..758: mol_type = other DNA
cggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccggctagcggggggctataaaagggggtgggggcgttcgtcctcactctagatctgcgatctaagtaagcttggcattccggtactgtaccggtgccaccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtgcatattgtccaaatgttcatcaacacttct

SEQ ID NO: 25 - DNA, 2147 bp, synthetic construct
  source 1..2147: mol_type = other DNA
cggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccggctagcggggggctataaaagggggtgggggcgttcgtcctcactctagatctgcgatctaagtaagcttggcattccggtactgtaccggtgccaccatgcggctctgcatcccgcaggtgctgttggccttgttcctttccatgctgacagggccgggagaaggcagctacccttacgatgtacctgattacgccggcggcggaggctcacaagtgcagcttcagcagagcggcgctgagctggcccggcccggggcctctgtgaaaatgagctgcaaggcttccggttacacctttacaaggtacacaatgcactgggtgaagcagcggccagggcaggggctcgagtggatcggctacataaacccatctcggggatatacaaactacaatcaaaagttcaaagacaaggccacactgacaacggacaagtcaagcagcacagcttacatgcaactgagctctctgacatccgaggatagcgccgtttactattgcgccagatactatgatgaccactattgcttggactactggggacagggaacaaccctcacagttagctccggcggggggggcagtggaggtggaggatctgggggcggcggtagtcagatcgtcctcacacagtctccggccataatgtccgcctcccccggagagaaggttactatgacatgttccgcatcttcctctgtgtcatatatgaattggtaccagcagaagagtggcacctctcctaaacgctggatttacgatacctctaaactggcgtccggggtgcctgcacatttccgcggatccggctccggtacgagttattcactcacaatatctggaatggaggccgaagatgccgctacttactactgccaacaatggtcaagcaaccccttcactttcgggagcgggacaaagctggagatcaaccaccatcatcatcaccatcatgcaggtatggcctcctctgggtatgtcctccaggcggagctctccccctcaactgagaactcaagtcagctggacttcgaagatgtatggaattcttcctatggtgtgaatgattccttcccagatggagactatgatgccaacctggaagcagctgccccctgccactcctgtaacctgctggatgactctgcactgcccttcttcatcctcaccagtgtcctgggtatcctagctagcagcactgtcctcttcatgcttttcagacctctcttccgctggcagctctgccctggctggcctgtcctggcacagctggctgtgggcagtgccctcttcagcattgtggtgcccgtcttggccccagggctaggtagcactcgcagctctgccctgtgtagcctgggctactgtgtctggtatggctcagcctttgcccaggctttgctgctagggtgccatgcctccctgggccacagactgggtgcaggccaggtcccaggcctcaccctggggctcactgtgggaatttggggagtggctgccctactgacactgcctgtcaccctggccagtggtgcttctggtggactctgcaccctgatatacagcacggagctgaaggctttgcaggccacacacactgtagcctgtcttgccatctttgtcttgttgccattgggtttgtttggagccaaggggctgaagaaggcattgggtatggggccaggcccctggatgaatatcctgtgggcctggtttattttctggtggcctcatggggtggttctaggactggatttcctggtgaggtccaagctgttgctgttgtcaacatgtctggcccagcaggctctggacctgctgctgaacctggcagaagccctggcaattttgcactgtgtggctacgcccctgctcctcgccctattctgccaccaggccacccgcaccctcttgccctctctgcccctccctgaaggatggtcttctcatctggacacccttggaagcaaatcc

SEQ ID NO: 26 - DNA, 880 bp, synthetic construct
  source 1..880: mol_type = other DNA
cggagtactgtcctccgagcggagtactgtcctccgactcgagcggagtactgtcctccgaagacgctagcggggggctataaaagggggtgggggcgttcgtcctcactctagatctgcgatctaagtaagcttggcattccggtactgtaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 27 - DNA, 989 bp, synthetic construct
  source 1..989: mol_type = other DNA
cggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccggctagcggggggctataaaagggggtgggggcgttcgtcctcactctagatctgcgatctaagtaagcttggcattccggtactgtaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 28 - DNA, 946 bp, synthetic construct
  source 1..946: mol_type = other DNA
cggagtactgtcctccgagcggagtactgtcctccgactcgagcggagtactgtcctccgaagacgctagcggggggctataaaagggggtgggggcgttcgtcctcactctagatctgcgatctaagtaagcttggcattccggtactgtaccggtgccaccatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactc

SEQ ID NO: 29 - DNA, 1055 bp, synthetic construct
  source 1..1055: mol_type = other DNA
cggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccggctagcggggggctataaaagggggtgggggcgttcgtcctcactctagatctgcgatctaagtaagcttggcattccggtactgtaccggtgccaccatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactc

SEQ ID NO: 30 - DNA, 25 bp, synthetic construct
  source 1..25: mol_type = other DNA
tctagagggtatataatgggggcca

SEQ ID NO: 31 - DNA, 46 bp, synthetic construct
  source 1..46: mol_type = other DNA
agatctgcgatctaagtaagcttggcattccggtactgttggtaaa

SEQ ID NO: 32 - DNA, 1212 bp, Homo sapiens
  source 1..1212: mol_type = genomic DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 33 - DNA, 441 bp, synthetic construct
  source 1..441: mol_type = other DNA
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcg

SEQ ID NO: 34 - protein, 147 aa, synthetic construct
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 35 - DNA, 141 bp, synthetic construct
  source 1..141: mol_type = other DNA
atgtcgacggcccccccgaccgatgtcagcctgggggacgagctccacttagacggcgaggacgtggcgatggcgcatgccgacgcgctagacgatttcgatctggacatgttgggggacggggattccccgggtccggga

SEQ ID NO: 36 - protein, 47 aa, synthetic construct
MSTAPPTDVSLGDELHLDGEDVAMAHADALDDFDLDMLGDGDSPGPG

SEQ ID NO: 37 - DNA, 414 bp, synthetic construct
  source 1..414: mol_type = other DNA
gtggtagtagaaattggcaaagttacgggatctgttggaactacagttgaaatacctgtatatttcagaggagttccatccaaaggaatagcaaactgcgactttgtgttcagatatgatccgaatgtattggaaattatagggatagatcccggagacataatagttgacccgaatcctaccaagagctttgatactgcaatatatcctgacagaaagataatagtattcctgtttgcggaagacagcggaacaggagcgtatgcaataactaaagacggagtatttgcaaaaataagagcaactgtaaaatcaagtgctccgggctatattactttcgacgaagtaggtggatttgcagataatgacctggtagaacagaaggtatcatttatagacggtggtgttaacgtt

SEQ ID NO: 38 - protein, 138 aa, synthetic construct
VVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNV

SEQ ID NO: 39 - DNA, 207 bp, synthetic construct
  source 1..207: mol_type = other DNA
tctactaaattatacggcgacgtcaatgatgacggaaaagttaactcaactgacgctgtagcattgaagagatatgttttgagatcaggtataagcatcaacactgacaatgccgatttgaatgaagacggcagagttaattcaactgacttaggaattttgaagagatatattctcaaagaaatagatacattgccgtacaagaac

SEQ ID NO: 40 - protein, 69 aa, synthetic construct
STKLYGDVNDDGKVNSTDAVALKRYVLRSGISINTDNADLNEDGRVNSTDLGILKRYILKEIDTLPYKN

SEQ ID NO: 41 - DNA, 903 bp, synthetic construct
  source 1..903: mol_type = other DNA
agtcttgatgaaaggtttgaggatgatctggacgaggagcgggttccgaatctggggatagttgccgaaagtatttcgcaactgggtatcgacgtgctgctatcgggtgagaccggcacgggcaaagacacgattgcccgacggattcatgagatgtcaggccgcaaagggcgcctggtggcgatgaattgcgcggccattccggagtccctcgccgagagcgagttattcggcgtggtcagcggtgcctacaccggcgctgatcgctccagagtcggttatgtcgaagcggcgcagggcggcacgctgtacctggatgagatcgatagcatgccgctgagcctgcaagccaaattgctgagggtgctggaaacccgagcgcttgaacggctgggttcgacgtcgacgatcaagctggatatctgcgtgatcgcctccgcccaatgctcgctggacgacgccgtcgagcgggggcagtttcgtcgcgatctgtattttcgcctgaacgtcctgacactcaagcttcctccgctacgtaaccagtctgatcgcatagttcccctgttcacacgttttacggccgccgccgcgagggagctcggtgttcccgttcccgatgtttgcccactgctgcacaaagtgctgctgggccacgactggcccggcaatatccgtgagctcaaggcggcagccaaacgccatgtgctgggtttccccttgctgggcgccgagccgcagggcgaagagcacttggcctgtgggctcaaatcgcaattgcgagtgatcgaaaaagccctgattcaggagtcgctcaagcgccacgacaattgtgtggattcggtaagcctggaactggacgtgccacgccgtacgctctatcgacgcatcaaagaattgcagatc

SEQ ID NO: 42 - protein, 301 aa, synthetic construct
SLDERFEDDLDEERVPNLGIVAESISQLGIDVLLSGETGTGKDTIARRIHEMSGRKGRLVAMNCAAIPESLAESELFGVVSGAYTGADRSRVGYVEAAQGGTLYLDEIDSMPLSLQAKLLRVLETRALERLGSTSTIKLDICVIASAQCSLDDAVERGQFRRDLYFRLNVLTLKLPPLRNQSDRIVPLFTRFTAAAARELGVPVPDVCPLLHKVLLGHDWPGNIRELKAAAKRHVLGFPLLGAEPQGEEHLACGLKSQLRVIEKALIQESLKRHDNCVDSVSLELDVPRRTLYRRIKELQI

SEQ ID NO: 43 - DNA, 939 bp, synthetic construct
  source 1..939: mol_type = other DNA
agtacaggcatcgataaggacgtccgagagtgttggggcgtaactgcattatcagcgggtcatcaaattgcaatgaatagcgcgtttctggatatggacttgctgttgtgcggggaaaccggcaccggcaaggacacactggccaaccgcattcacgagttgtccagcaggtcgggaccctttgtgggcatgaactgcgccgccattcccgagtcgctggcagagagccagttattcggtgtggtcaacggtgcattcaccggcgtatgccgggctcgcgagggctacatagaggcctccagtggtggcaccttgtacctggatgaaatcgacagcatgccgttgagcctgcaagccaaactgctgcgtgtgttggagagtcgaggtatcgagcgtctgggctcgaccgaatttatcccggtggatctgcggatcattgcctcggcccagcggccactggatgaactggtggaacaaggacttttccgtcgcgacctgttttttcggctcaacgtgctgacgcttcacttgccagccttgcgcaaacgtcgtgaacagatcctgccattgttcgaccagttcacccagggtatcgctgccgagttcggacgtcccgctcctgcgctggacagcgggcgtgtgcagctgctgctcagccacgactggccgggcaacatccgcgaattgaagtctgcggccaagcgcttcgtactcggcttccccttgctgggcgccgaccctgtggaagcgcttgaccctgccacggggctgcgcacgcaaatgcgcatcatcgagaaaatgctcatccaggatgccttgaagcggcacaggcacaatttcgacgcggtgcttcaggagttggagttgccaagacgcaccctgtatcaccgcatgaaggaactgggagttgcagcgccgatcgctgcgacggccggggtc

SEQ ID NO: 44 - protein, 313 aa, synthetic construct
STGIDKDVRECWGVTALSAGHQIAMNSAFLDMDLLLCGETGTGKDTLANRIHELSSRSGPFVGMNCAAIPESLAESQLFGVVNGAFTGVCRAREGYIEASSGGTLYLDEIDSMPLSLQAKLLRVLESRGIERLGSTEFIPVDLRIIASAQRPLDELVEQGLFRRDLFFRLNVLTLHLPALRKRREQILPLFDQFTQGIAAEFGRPAPALDSGRVQLLLSHDWPGNIRELKSAAKRFVLGFPLLGADPVEALDPATGLRTQMRIIEKMLIQDALKRHRHNFDAVLQELELPRRTLYHRMKELGVAAPIAATAGV

SEQ ID NO: 45 - DNA, 174 bp, synthetic construct
  source 1..174: mol_type = other DNA
cggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccg

SEQ ID NO: 46 - DNA, 60 bp, synthetic construct
  source 1..60: mol_type = other DNA
cggagtactgtcctccgagcggagtactgtcctccgactcgagcggagtactgtcctccg

SEQ ID NO: 47 - DNA, 399 bp, synthetic construct
  source 1..399: mol_type = other DNA
ggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtgcatattgtccaaatgttcatcaacacttct

SEQ ID NO: 48 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 49 - DNA, 399 bp, synthetic construct
  source 1..399: mol_type = other DNA
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 50 - protein, 133 aa, Homo sapiens [sequence omitted]

SEQ ID NO: 51 - DNA, 720 bp, synthetic construct
  source 1..720: mol_type = other DNA
caagtgcagcttcagcagagcggcgctgagctggcccggcccggggcctctgtgaaaatgagctgcaaggcttccggttacacctttacaaggtacacaatgcactgggtgaagcagcggccagggcaggggctcgagtggatcggctacataaacccatctcggggatatacaaactacaatcaaaagttcaaagacaaggccacactgacaacggacaagtcaagcagcacagcttacatgcaactgagctctctgacatccgaggatagcgccgtttactattgcgccagatactatgatgaccactattgcttggactactggggacagggaacaaccctcacagttagctccggcggggggggcagtggaggtggaggatctgggggcggcggtagtcagatcgtcctcacacagtctccggccataatgtccgcctcccccggagagaaggttactatgacatgttccgcatcttcctctgtgtcatatatgaattggtaccagcagaagagtggcacctctcctaaacgctggatttacgatacctctaaactggcgtccggggtgcctgcacatttccgcggatccggctccggtacgagttattcactcacaatatctggaatggaggccgaagatgccgctacttactactgccaacaatggtcaagcaaccccttcactttcgggagcgggacaaagctggagatcaac

SEQ ID NO: 52 - protein, 240 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 53 - DNA, 1020 bp, synthetic construct
  source 1..1020: mol_type = other DNA
gcaggtatggcctcctctgggtatgtcctccaggcggagctctccccctcaactgagaactcaagtcagctggacttcgaagatgtatggaattcttcctatggtgtgaatgattccttcccagatggagactatgatgccaacctggaagcagctgccccctgccactcctgtaacctgctggatgactctgcactgcccttcttcatcctcaccagtgtcctgggtatcctagctagcagcactgtcctcttcatgcttttcagacctctcttccgctggcagctctgccctggctggcctgtcctggcacagctggctgtgggcagtgccctcttcagcattgtggtgcccgtcttggccccagggctaggtagcactcgcagctctgccctgtgtagcctgggctactgtgtctggtatggctcagcctttgcccaggctttgctgctagggtgccatgcctccctgggccacagactgggtgcaggccaggtcccaggcctcaccctggggctcactgtgggaatttggggagtggctgccctactgacactgcctgtcaccctggccagtggtgcttctggtggactctgcaccctgatatacagcacggagctgaaggctttgcaggccacacacactgtagcctgtcttgccatctttgtcttgttgccattgggtttgtttggagccaaggggctgaagaaggcattgggtatggggccaggcccctggatgaatatcctgtgggcctggtttattttctggtggcctcatggggtggttctaggactggatttcctggtgaggtccaagctgttgctgttgtcaacatgtctggcccagcaggctctggacctgctgctgaacctggcagaagccctggcaattttgcactgtgtggctacgcccctgctcctcgccctattctgccaccaggccacccgcaccctcttgccctctctgcccctccctgaaggatggtcttctcatctggacacccttggaagcaaatcc

SEQ ID NO: 54 - protein, 340 aa, synthetic construct
AGMASSGYVLQAELSPSTENSSQLDFEDVWNSSYGVNDSFPDGDYDANLEAAAPCHSCNLLDDSALPFFILTSVLGILASSTVLFMLFRPLFRWQLCPGWPVLAQLAVGSALFSIVVPVLAPGLGSTRSSALCSLGYCVWYGSAFAQALLLGCHASLGHRLGAGQVPGLTLGLTVGIWGVAALLTLPVTLASGASGGLCTLIYSTELKALQATHTVACLAIFVLLPLGLFGAKGLKKALGMGPGPWMNILWAWFIFWWPHGVVLGLDFLVRSKLLLLSTCLAQQALDLLLNLAEALAILHCVATPLLLALFCHQATRTLLPSLPLPEGWSSHLDTLGSKS

SEQ ID NO: 55 - DNA, 87 bp, synthetic construct
  source 1..87: mol_type = other DNA
atgagaatttcgaaaccacatttgagaagtatttccatccagtgctacttgtgtttacttctaaacagtcattttctaactgaagct

SEQ ID NO: 56 - protein, 29 aa, synthetic construct
MRISKPHLRSISIQCYLCLLLNSHFLTEA

SEQ ID NO: 57 - DNA, 60 bp, synthetic construct
  source 1..60: mol_type = other DNA
atgtacaggatgcaactcctgtcttgcattgcactaagtcttgcacttgtcacaaacagt

SEQ ID NO: 58 - protein, 20 aa, synthetic construct
MYRMQLLSCIALSLALVTNS

SEQ ID NO: 59 - DNA, 72 bp, synthetic construct
  source 1..72: mol_type = other DNA
atgcggctctgcatcccgcaggtgctgttggccttgttcctttccatgctgacagggccgggagaaggcagc

SEQ ID NO: 60 - protein, 24 aa, synthetic construct
MRLCIPQVLLALFLSMLTGPGEGS

SEQ ID NO: 61 - DNA, 357 bp, synthetic construct
  source 1..357: mol_type = other DNA
atgatcgaggtgaccgagaagagcgccttctacgcccaagtggccgctcagagccctgctgtttggcctgtggctaatggcgtggcctttgtgtccagaagggaacaccatgattggggcatcgccctgcacatcgagggaagagccctccgccccgaccagctgagagatgccctgcagagacggtttatggaaagcgaaagattcaaccactacttcctgttcctggatgtgcggcgggacttcgtggtctggcacgccgtgaacgagaagccaggcagctacgccagcctggacgacatcagaagacacgagctgatgctggccggcctggaccacctgtctgaggaaatgcac

SEQ ID NO: 62 - protein, 119 aa, synthetic construct
MIEVTEKSAFYAQVAAQSPAVWPVANGVAFVSRREHHDWGIALHIEGRALRPDQLRDALQRRFMESERFNHYFLFLDVRRDFVVWHAVNEKPGSYASLDDIRRHELMLAGLDHLSEEMH

SEQ ID NO: 63 - DNA, 761 bp, synthetic construct
  source 1..761: mol_type = other DNA
cggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccggctagcggggggctataaaagggggtgggggcgttcgtcctcactctagatctgcgatctaagtaagcttggcattccggtactgtaccggtgccaccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtgcatattgtccaaatgttcatcaacacttcttga

SEQ ID NO: 64 - DNA, 734 bp, synthetic construct
  source 1..734: mol_type = other DNA
cggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccggctagcggggggctataaaagggggtgggggcgttcgtcctcactctagatctgcgatctaagtaagcttggcattccggtactgtaccggtgccaccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 65 - DNA, 2150 bp, synthetic construct
  source 1..2150: mol_type = other DNA
cggagtactgtcctccgagcggagtactgtcctccgactcgagcggagtactgtcctccgatcggagtactgtcctccgcgaattccggagtactgtcctccgaagacgctagacggagtactgtcctccgagcggagtactgtcctccgactcgagcggagtactgtcctccggctagcggggggctataaaagggggtgggggcgttcgtcctcactctagatctgcgatctaagtaagcttggcattccggtactgtaccggtgccaccatgcggctctgcatcccgcaggtgctgttggccttgttcctttccatgctgacagggccgggagaaggcagctacccttacgatgtacctgattacgccggcggcggaggctcacaagtgcagcttcagcagagcggcgctgagctggcccggcccggggcctctgtgaaaatgagctgcaaggcttccggttacacctttacaaggtacacaatgcactgggtgaagcagcggccagggcaggggctcgagtggatcggctacataaacccatctcggggatatacaaactacaatcaaaagttcaaagacaaggccacactgacaacggacaagtcaagcagcacagcttacatgcaactgagctctctgacatccgaggatagcgccgtttactattgcgccagatactatgatgaccactattgcttggactactggggacagggaacaaccctcacagttagctccggcggggggggcagtggaggtggaggatctgggggcggcggtagtcagatcgtcctcacacagtctccggccataatgtccgcctcccccggagagaaggttactatgacatgttccgcatcttcctctgtgtcatatatgaattggtaccagcagaagagtggcacctctcctaaacgctggatttacgatacctctaaactggcgtccggggtgcctgcacatttccgcggatccggctccggtacgagttattcactcacaatatctggaatggaggccgaagatgccgctacttactactgccaacaatggtcaagcaaccccttcactttcgggagcgggacaaagctggagatcaaccaccatcatcatcaccatcatgcaggtatggcctcctctgggtatgtcctccaggcggagctctccccctcaactgagaactcaagtcagctggacttcgaagatgtatggaattcttcctatggtgtgaatgattccttcccagatggagactatgatgccaacctggaagcagctgccccctgccactcctgtaacctgctggatgactctgcactgcccttcttcatcctcaccagtgtcctgggtatcctagctagcagcactgtcctcttcatgcttttcagacctctcttccgctggcagctctgccctggctggcctgtcctggcacagctggctgtgggcagtgccctcttcagcattgtggtgcccgtcttggccccagggctaggtagcactcgcagctctgccctgtgtagcctgggctactgtgtctggtatggctcagcctttgcccaggctttgctgctagggtgccatgcctccctgggccacagactgggtgcaggccaggtcccaggcctcaccctggggctcactgtgggaatttggggagtggctgccctactgacactgcctgtcaccctggccagtggtgcttctggtggactctgcaccctgatatacagcacggagctgaaggctttgcaggccacacacactgtagcctgtcttgccatctttgtcttgttgccattgggtttgtttggagccaaggggctgaagaaggcattgggtatggggccaggcccctggatgaatatcctgtgggcctggtttattttctggtggcctcatggggtggttctaggactggatttcctggtgaggtccaagctgttgctgttgtcaacatgtctggcccagcaggctctggacctgctgctgaacctggcagaagccctggcaattttgcactgtgtggctacgcccctgctcctcgccctattctgccaccaggccacccgcaccctcttgccctctctgcccctccctgaaggatggtcttctcatctggacacccttggaagcaaatcctaa

SEQ ID NO: 66 - DNA, 783 bp, synthetic construct
  source 1..783: mol_type = other DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactc

SEQ ID NO: 67 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 68 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - DNA, 345 bp, synthetic construct
  source 1..345: mol_type = other DNA
gccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 71 - protein, 115 aa, Homo sapiens
ANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 72 - DNA, 60 bp, synthetic construct
  source 1..60: mol_type = other DNA
atggtattgggaaccatagatttgtgcagctgtttcagtgcagggcttcctaaaacagaa

SEQ ID NO: 73 - protein, 20 aa, synthetic construct
MVLGTIDLCSCFSAGLPKTE

SEQ ID NO: 74 - DNA, 381 bp, synthetic construct
  source 1..381: mol_type = other DNA
gcacctgccagatctcctagtcccagcacacaaccctgggagcatgtgaatgccatccaggaggccaggagactcctgaacctgagtagagatactgctgctgagatgaatgaaacagtggaagttatctctgaaatgtttgacctccaagagcctacttgccttcagaccagactggagctgtataagcagggactgcggggcagcctcaccaagctcaagggccccttgaccatgatggctagccactacaaacagcactgccctccaacaccagaaacttcctgtgcaacccagattatcacctttgaaagtttcaaagagaacctgaaggacttcctgcttgtcattccctttgactgctgggagccagtgcaggag

SEQ ID NO: 75 - protein, 127 aa, Homo sapiens [sequence omitted]

SEQ ID NO: 76 - DNA, 51 bp, synthetic construct
  source 1..51: mol_type = other DNA
atgtggctgcagagcctgctgctcttgggcactgtggcctgtagcatctct

SEQ ID NO: 77 - protein, 17 aa, synthetic construct
MWLQSLLLLGTVACSIS

SEQ ID NO: 78 - DNA, 627 bp, synthetic construct
  source 1..627: mol_type = other DNA
acccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcac

SEQ ID NO: 79 - protein, 209 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 80 - DNA, 78 bp, synthetic construct
  source 1..78: mol_type = other DNA
atgacagtgctggcgccagcctggagcccaacaacctatctcctcctgctgctgctgctgagctcgggactcagtggg

SEQ ID NO: 81 - protein, 26 aa, synthetic construct
MTVLAPAWSPTTYLLLLLLLSSGLSG

SEQ ID NO: 82 - DNA, 2124 bp, synthetic construct
  source 1..2124: mol_type = other DNA
atgggagttaaggtattgttcgcattgatttgcatagccgtggccgaagctcaggtgcagctcgtagagagcgggggaggtgtcgttcagccagggaggagtctgcggctggactgcaaagcctccgggattactttctcaaacagcgggatgcactgggtgaggcaggctcccggaaagggcctcgagtgggtggcggtaatatggtacgacgggagcaaacgctactacgcagacagtgtgaaaggaaggttcactatttctcgcgacaattcaaagaacaccctcttcctgcagatgaacagtctgcgggcagaagacaccgctgtctattactgtgccaccaatgacgattactggggccagggtaccctcgtgaccgtgtctagcgcctctaccaaaggaccatcagtttttcctcttgctccctgcagtcgctcaaccagcgagagcacagcagcgctgggatgtctggtgaaggattattttcctgagcccgtgactgtgagctggaattcaggcgccctgacgtccggcgtccatacattcccagcggtacttcaaagtagcgggttgtactctctctctagcgtggtaaccgtaccgagctcctccctggggacgaaaacgtatacatgtaatgtcgatcacaaaccatctaacacaaaagtggacaaacgcgttgagtccaagtatggccctccatgcccaccctgccccgcaccggagtttctgggcgggcccagtgtctttctgttcccaccgaagcctaaggacacgttgatgatctcaagaacacctgaagtcacctgcgtagtcgtggacgtttctcaggaggatcccgaggtccaattcaattggtacgttgatggagtggaggtccacaacgcaaagacaaagccgcgcgaagaacagttcaattctacttaccgcgttgttagcgtgctgactgtgctccaccaagactggctgaatggtaaggagtataagtgcaaggtgagcaataagggattgccatctagcatcgaaaagacaatatccaaagccaagggccaaccacgagagccacaagtgtacacgttgcctccctcacaagaagagatgaccaagaatcaagtgagcctcacttgcctggtcaagggattctacccttctgatatcgcagtggagtgggagtccaatggacagcccgagaacaactacaagacaacacccccagtgctggattccgacggctcattcttcttgtatagccggctgacagtggacaagagcaggtggcaggaaggaaatgtcttctcctgctccgtgatgcacgaggccctccacaaccactacactcagaaatctctctctctttcactcggtaaaggaagcggagctactaacttcagcctgctgaagcaggctggagacgtggaggagaaccctggacctggagttaaggtattgttcgcattgatttgcatagccgtggccgaagctgaaatagtgctgacccagtcccccgccaccctctctctctcacccggagagagggcaactctgtcctgcagggcctcccaatccgtaagcagctatctggcttggtaccagcaaaaacccggacaggcaccccggctgttgatctatgacgcctccaaccgagccactggcatccccgccagattctccggatctggatcagggactgattttaccctgactatcagttctctggaacctgaagattttgcagtttattattgtcagcaaagctctaactggccaagaacctttggccagggcacgaaggtcgagataaaaactgtcgcggccccaagcgtctttatctttccgccgtctgatgaacagctgaaatcagggaccgcctctgtggtgtgtctgctgaacaacttctacccaagggaagctaaggtgcaatggaaagtagacaatgccttgcagagcggcaactctcaggagagcgtaaccgaacaggatagtaaggattcaacttacagtctgagctctaccctgactctgagtaaggccgattacgagaagcataaagtctacgcatgcgaggtcacacatcaaggtctttcatctcccgtgacaaagtcctttaatcgaggggaatgc

SEQ ID NO: 83 - protein, 708 aa, synthetic construct
MGVKVLFALICIAVAEAQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGATNFSLLKQAGDVEENPGPGVKVLFALICIAVAEAEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - DNA, 51 bp, synthetic construct
  source 1..51: mol_type = other DNA
atgggagttaaggtattgttcgcattgatttgcatagccgtggccgaagct

SEQ ID NO: 85 - protein, 17 aa, synthetic construct
MGVKVLFALICIAVAEA

SEQ ID NO: 86 - DNA, 333 bp, synthetic construct
  source 1..333: mol_type = other DNA
agcgatggtggcgcacaggactgttgcctgaagtattcacagcggaaaatcccagctaaagtggttcggagctatcgcaagcaggaaccttccttggggtgttcaatcccagctatcctctttcttccaagaaagcgctctcaggccgagctgtgtgctgacccaaaagagctgtgggtccagcagttgatgcagcacttggacaagacgccgtctccccagaaacccgcccagggctgcagaaaagataggggcgcctcaaaaacaggcaaaaaagggaaaggtagcaaaggttgcaaacgcaccgaacgaagccagacgccaaaagggcca

SEQ ID NO: 87 - protein, 111 aa, Homo sapiens
SDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 88 - DNA, 69 bp, synthetic construct
  source 1..69: mol_type = other DNA
atggctcaatccttggcgctgtccttgctgatcttggtgctggcatttggcattccccgcacacaagga

SEQ ID NO: 89 - protein, 23 aa, synthetic construct
MAQSLALSLLILVLAFGIPRTQG

SEQ ID NO: 90 - DNA, 1077 bp, synthetic construct
  source 1..1077: mol_type = other DNA
ccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttctgctgttgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcc

SEQ ID NO: 91 - protein, 359 aa, Homo sapiens
PHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 92 - DNA, 72 bp, synthetic construct
  source 1..72: mol_type = other DNA
atggggctgggcccggtcttcctgcttctggctggcatcttcccttttgcacctccgggagctgctgctgag

SEQ ID NO: 93 - protein, 24 aa, synthetic construct
MGLGPVFLLLAGIFPFAPPGAAAE

SEQ ID NO: 94 - DNA, 657 bp, synthetic construct
  source 1..657: mol_type = other DNA
ggatgggtcgacacacactgtctttgctatgacttcatcatcactcctaagtccagacctgaaccacagtggtgtgaagttcaaggcctggtggatgaaaggccttttcttcactatgactgtgttaaccacaaggccaaagcctttgcttctctggggaagaaagtcaatgtcacaaaaacctgggaagaacaaactgaaacactaagagacgtggtggatttccttaaagggcaactgcttgacattcaagtggagaatttaatacccattgagcccctcaccctgcaggccaggatgtcttgtgagcatgaagcccatggacacggcagaggatcttggcagttcctcttcaatggacagaagttcctcctctttgactcaaacaacagaaagtggacagcacttcatcctggagccaagaagatgacagagaagtgggagaagaacagggatgtgaccatgttcttccagaagatttcactgggggattgtaagatgtggcttgaagaatttttgatgtactgggaacaaatgctggatccaacaaaaccaccctctctggccccaggcacaacccaacccaaggccatggccaccaccctcagtccctggagccttctcatcatcttcctctgcttcattctagctggcaga

SEQ ID NO: 95 - protein, 219 aa, Homo sapiens
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 96 - DNA, 75 bp, synthetic construct
  source 1..75: mol_type = other DNA
atggcagcggccgccagccccgcgttccttctgtgcctcccgcttctgcacctgctgtctggctggtcccgggca

SEQ ID NO: 97 - protein, 25 aa, synthetic construct
MAAAASPAFLLCLPLLHLLSGWSRA

SEQ ID NO: 98 - DNA, 762 bp, synthetic construct
  source 1..762: mol_type = other DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 99 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 100 - DNA, 480 bp, synthetic construct
  source 1..480: mol_type = other DNA
ctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctt

SEQ ID NO: 101 - protein, 160 aa, Homo sapiens
LLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 102 - DNA, 69 bp, synthetic construct
  source 1..69: mol_type = other DNA
atggaaagggtccaacccctggaagagaatgtgggaaatgcagccaggccaagattcgagaggaacaag

SEQ ID NO: 103 - protein, 23 aa, synthetic construct
MERVQPLEENVGNAARPRFERNK

SEQ ID NO: 104-105 - skipped (2 entries)

SEQ ID NO: 106 - DNA, 12 bp, synthetic construct
  source 1..12: mol_type = other DNA
ybhcybhdwthw

SEQ ID NO: 107 - DNA, 12 bp, synthetic construct
  source 1..12: mol_type = other DNA
tbhctbhdatht

SEQ ID NO: 108 - DNA, 11 bp, synthetic construct
  source 1..11: mol_type = other DNA
yctttgatsty

SEQ ID NO: 109 - DNA, 11 bp, synthetic construct
  source 1..11: mol_type = other DNA
cctttgatstt

SEQ ID NO: 110 - skipped

SEQ ID NO: 111 - DNA, 15 bp, synthetic construct
  source 1..15: mol_type = other DNA
nrasatcaaagnnww

SEQ ID NO: 112 - skipped

SEQ ID NO: 113 - DNA, 14 bp, synthetic construct
  source 1..14: mol_type = other DNA
racsgttaacskhy

SEQ ID NO: 114 - DNA, 14 bp, synthetic construct
  source 1..14: mol_type = other DNA
aacsgttaacsghy

SEQ ID NO: 115 - skipped